(19) United States
(12) Patent Application Publication
(10) Pub. No.: US 2021/0179684 A1
(43) Pub. Date: Jun. 17, 2021

(54) NEUROTROPHIN MUTANTS AND USE THEREOF FOR TREATING NEURODEGENERATIVE DISEASES AND DISORDERS

(71) Applicant: Horacio Uri SARAGOVI, Montreal (CA)

(72) Inventor: Horacio Uri SARAGOVI, Montreal (CA)

(21) Appl. No.: 16/481,312

(22) PCT Filed: Jan. 26, 2018
(86) PCT No.: PCT/US2018/015562

§ 371 Date: Jul. 26, 2019

Related U.S. Application Data
(60) Provisional application No. 62/451,560, filed on Jan. 27, 2017.

(51) Int. Cl. C07K 14/48; C07K 14/475

(52) U.S. Cl. CPC C07K 14/48; C07K 14/475

(57) ABSTRACT

Disclosed herein are compositions and methods for the treatment of non-otic diseases or conditions with Trk receptor agonist compositions and formulations.

### CROSS-REFERENCE

This application claims the benefit of U.S. Provisional Application No. 62/451,560 filed on Jan. 27, 2017, which is incorporated herein by reference in its entirety.

### BACKGROUND OF THE INVENTION

Neuron loss such as motor neuron loss is the cause of morbidity and mortality in many neurodegenerative diseases and spinal cord traumatic pathologies such as amyotrophic lateral sclerosis (ALS), spinal muscular atrophy (SMA), or spinal cord injury (SCI). In some instances, motor neurons express Trk family of receptors such as TrkA, TrkB, and TrkC. Activity of the Trk family of receptors, mediated by the kinase catalytic domain, correlates with neuron survival or death, maintenance of synapses and phenotype, and function. As such, modulation of one or more Trk family of receptors or p75^{NTR}, in some instances, further modulates a neurodegenerative disease or disorder.

### SUMMARY OF THE INVENTION

Described herein, in certain embodiments, are neurotrophic agents comprising modifications at amino acid residue positions equivalent to amino acid residues 7 and 103 set forth in SEQ ID NO: 1. In some embodiments, the neurotrophic agent further comprises a modification at an amino acid residue position equivalent to amino acid residue 84 set forth in SEQ ID NO: 1. In some embodiments, the neurotrophic agent further comprises a modification at an amino acid residue position equivalent to amino acid residue 45 set forth in SEQ ID NO: 1. In some embodiments, the neurotrophic agent further comprises modifications at amino acid residue positions equivalent to amino acid residues 32, 34, and 35 set forth in SEQ ID NO: 1. In some embodiments, the neurotrophic agent comprises nerve growth factor (NGF), neurotrophin-3 (NT-3), neurotrophin-4 (NT-4), neurotrophin-5 (NT-5), or brain-derived neurotrophic factor (BDNF). In some embodiments, the neurotrophic agent comprises a pro-form of nerve growth factor (proNGF), neurotrophin-3 (proNT-3), neurotrophin-4 (proNT-4), neurotrophin-5 (proNT-5), or brain-derived neurotrophic factor (proBDNF). In some embodiments, the neurotrophic agent is NGF. In some embodiments, NGF comprises modifications at amino acid residue positions equivalent to amino acid residue 7, 84, and 103 set forth in SEQ ID NO: 1. In some embodiments, NGF comprises modifications at amino acid residue positions equivalent to amino acid residue 7, 45, and 103 set forth in SEQ ID NO: 1. In some embodiments, NGF further comprises modifications at amino acid residue positions equivalent to amino acid residues 32, 34, and 35 set forth in SEQ ID NO: 1. In some embodiments, the modification comprises a mutation to a non-polar residue. In some embodiments, the modification comprises a mutation to a polar residue. In some embodiments, the modification comprises a mutation to a charged residue. In some embodiments, the modification comprises a mutation to alanine. In some embodiments, the modification comprises a mutation to a conservative amino acid. In some embodiments, the modification comprises a mutation to a non-conservative amino acid. In some embodiments, the modification comprises a mutation to a semi-conservative amino acid. In some embodiments, NGF comprises about 70%, 80%, 85%, 90%, 91%, 92%, 93%, 94%, 95%, 96%, 97%, 98%, or 99% sequence identity to SEQ ID NO: 8, 9, 10 or 11. In some embodiments, NGF comprises the amino acid sequence set forth in SEQ ID NO: 8, 9, 10, or 11. In some embodiments, the neurotrophic agent is NT-3. In some embodiments, the neurotrophic agent is NT-4 or NT-5. In some embodiments, the neurotrophic agent is BDNF. In some embodiments, the neurotrophic agent is a pan-neurotrophin (PNT). In some embodiments, the neurotrophic agent is PNT-1. In some embodiments, the neurotrophic agent comprises a sequence set forth in SEQ ID NO: 6. In some embodiments, the neurotrophic agent comprises a sequence set forth in SEQ ID NO: 7.

Described herein, in certain embodiments, are neurotrophic agents comprising at least two modifications selected from amino acid residue positions equivalent to amino acid residues 74, 75, and 77 set forth in SEQ ID NO: 5. In some embodiments, the neurotrophic agent further comprises a modification at an amino acid residue position equivalent to amino acid residue 32, 34, 115, or 116 set forth in SEQ ID NO: 5. In some embodiments, the neurotrophic agent comprises nerve growth factor (NGF), neurotrophin-3 (NT-3), neurotrophin-4 (NT-4), neurotrophin-5 (NT-5), or brain-derived neurotrophic factor (BDNF). In some embodiments, the neurotrophic agent is a pan-neurotrophin (PNT). In some embodiments, PNT comprises modifications at amino acid residue positions equivalent to amino acid residues 74, 75, and 77 set forth in SEQ ID NO: 5. In some embodiments, PNT further comprises modifications at amino acid residue positions equivalent to amino acid residues 32, 34, 115, or 116 set forth in SEQ ID NO: 5. In some embodiments, the modifications comprise a mutation to a non-polar residue. In some embodiments, modifications comprise a mutation to a polar residue. In some embodiments, the modifications comprise a mutation to a charged residue. In some embodiments, the modifications comprise a mutation to alanine. In some embodiments, the neurotrophic agent comprises about 70%, 80%, 85%, 90%, 91%, 92%, 93%, 94%, 95%, 96%, 97%, 98% or 99% sequence identity to SEQ ID NO: 14 or 16. In some embodiments, the neurotrophic agent comprises the amino acid sequence set forth in SEQ ID NO: 14 or 16. In some embodiments, the neurotrophic agent is NGF. In some embodiments, the neurotrophic agent is NT-3. In some embodiments, the neurotrophic agent is NT-4 or NT-5. In some embodiments, the neurotrophic agent is BDNF. In some embodiments, the neurotrophic agent comprises a sequence set forth in SEQ ID NO: 6. In some embodiments, the neurotrophic agent comprises a sequence set forth in SEQ ID NO: 7.

Described herein, in certain embodiments, are neurotrophic agents comprising modifications at amino acid residue positions equivalent to amino acid residues 32 and 34 set forth in SEQ ID NO: 5, wherein the neurotrophic agent is selected from NGF, NT-4, NT-5, BDNF, or PNT. In some embodiments, the neurotrophic agent further comprises a modification at an amino acid residue position equivalent to amino acid residue 74, 75, 77, 115, or 116 set forth in SEQ ID NO: 5. In some embodiments, the neurotrophic agent is PNT. In some embodiments, PNT comprises modifications at amino acid residue positions equivalent to amino acid residues 32 and 34 set forth in SEQ ID NO: 5. In some embodiments, PNT further comprises modifications at amino acid residue positions equivalent to amino acid residues 74, 75, 77, 115, and 116 set forth in SEQ ID NO: 5. In some embodiments, the modification comprises a mutation to a non-polar residue. In some embodiments, the modification comprises a mutation to a polar residue. In some embodiments, the modification comprises a mutation to a charged residue. In some embodiments, the modification comprises a mutation to alanine. In some embodiments, the neurotrophic agent comprises about 70%, 80%, 85%, 90%, 91%, 92%, 93%, 94%, 95%, 96%, 97%, 98%, or 99% sequence identity to SEQ ID NO: 13 or 16. In some embodiments, the neurotrophic agent comprises the amino acid sequence set forth in SEQ ID NO: 13 or 16. In some embodiments, the neurotrophic agent is NGF. In some embodiments, the neurotrophic agent is NT-4 or NT-5. In some embodiments, the neurotrophic agent is BDNF. In some embodiments, the neurotrophic agent comprises a sequence set forth in SEQ ID NO: 6. In some embodiments, the neurotrophic agent comprises a sequence set forth in SEQ ID NO: 7. In some embodiments, the neurotrophic agent is a pan-neurotrophin.

Described herein, in certain embodiments, are neurotrophic agents comprising modifications at amino acid residue positions equivalent to amino acid residues 115 and 116 set forth in SEQ ID NO: 5, wherein the neurotrophic agent is selected from NGF, NT-4, NT-5, BDNF, or PNT. In some embodiments, the neurotrophic agent further comprises a modification at an amino acid residue position equivalent to amino acid residue 32, 34, 74, 75, or 77 set forth in SEQ ID NO: 5. In some embodiments, the neurotrophic agent is PNT. In some embodiments, PNT comprises modifications at amino acid residue positions equivalent to amino acid residues 115 and 116 set forth in SEQ ID NO: 5. In some embodiments, PNT comprises further modifications at amino acid residue positions equivalent to amino acid residues 32, 34, 74, 75, and 77 set forth in SEQ ID NO: 5. In some embodiments, the modification comprises a mutation to a non-polar residue. In some embodiments, the modification comprises a mutation to a polar residue. In some embodiments, the modification comprises a mutation to a charged residue. In some embodiments, the modification comprises a mutation to alanine. In some embodiments, the neurotrophic agent comprises about 70%, 80%, 85%, 90%, 91%, 92%, 93%, 94%, 95%, 96%, 97%, 98%, or 99% sequence identity to SEQ ID NO: 15 or 16. In some embodiments, the neurotrophic agent comprises the amino acid sequence set forth in SEQ ID NO: 15 or 16. In some embodiments, the neurotrophic agent is NGF. In some embodiments, the neurotrophic agent is NT-4 or NT-5. In some embodiments, the neurotrophic agent is BDNF. In some embodiments, the neurotrophic agent comprises a sequence set forth in SEQ ID NO: 6. In some embodiments, the neurotrophic agent comprises a sequence set forth in SEQ ID NO: 7. In some embodiments, the neurotrophic agent is a pan-neurotrophin.

Described herein, in certain embodiments, are neurotrophic agents comprising modifications at amino acid residue positions equivalent to amino acid residues 61 and 100 set forth in SEQ ID NO: 5, wherein the neurotrophic agent is selected from NT-3, NT-4, NT-5, BDNF, or PNT. In some embodiments, the neurotrophic agent is PNT. In some embodiments, PNT comprises modifications at amino acid residue positions equivalent to amino acid residues 61 and 100 set forth in SEQ ID NO: 5. In some embodiments, the modification comprises a mutation to a non-polar residue. In some embodiments, the modification comprises a mutation to a polar residue. In some embodiments, the modification comprises a mutation to a charged residue. In some embodiments, the modification at amino acid residue 61 comprises a mutation from proline to serine. In some embodiments, the modification at amino acid residue 100 comprises a mutation from arginine to glutamic acid. In some embodiments, the neurotrophic agent comprises about 70%, 80%, 85%, 90%, 91%, 92%, 93%, 94%, 95%, 96%, 97%, 98% or 99% sequence identity to SEQ ID NO: 20. In some embodiments, the neurotrophic agent comprises the amino acid sequence set forth in SEQ ID NO: 20. In some embodiments, the neurotrophic agent is NT-3. In some embodiments, the neurotrophic agent is NT-4 or NT-5. In some embodiments, the neurotrophic agent is BDNF. In some embodiments, the neurotrophic agent comprises a sequence set forth in SEQ ID NO: 6. In some embodiments, the neurotrophic agent comprises a sequence set forth in SEQ ID NO: 7. In some embodiments, the neurotrophic agent is a pan-neurotrophin.

Described herein, in certain embodiments, are neurotrophic agents comprising a modification at amino acid residue position equivalent to amino acid residue 118 set forth in SEQ ID NO: 5, wherein the neurotrophic agent is selected from NT-3, NT-4, NT-5, BDNF or PNT. In some embodiments, the neurotrophic agent is PNT. In some embodiments, PNT comprises a modification at amino acid residue positions equivalent to amino acid residue 118 set forth in SEQ ID NO: 5. In some embodiments, the modification comprises a mutation to a non-polar residue. In some embodiments, the modification comprises a mutation to a polar residue. In some embodiments, the modification comprises a mutation to a charged residue. In some embodiments, the modification comprises a mutation to alanine. In some embodiments, the neurotrophic agent comprises about 70%, 80%, 85%, 90%, 91%, 92%, 93%, 94%, 95%, 96%, 97%, 98% or 99% sequence identity to SEQ ID NO: 21. In some embodiments, the neurotrophic agent comprises the amino acid sequence set forth in SEQ ID NO: 21. In some embodiments, the neurotrophic agent is NT-3. In some embodiments, the neurotrophic agent is NT-4 or NT-5. In some embodiments, the neurotrophic agent is BDNF. In some embodiments, the neurotrophic agent comprises a sequence set forth in SEQ ID NO: 6. In some embodiments, the neurotrophic agent comprises a sequence set forth in SEQ ID NO: 7. In some embodiments, the neurotrophic agent is a pan-neurotrophin.

Described herein, in certain embodiments, are neurotrophic agents comprising a sequence set forth in SEQ ID NOs: 17-19. In some embodiments, the neurotrophic agent recognizes one or more tropomyosin receptor kinase (Trk) receptors or their isoforms. In some embodiments, the neurotrophic agent recognizes p75^{NTR} or the p75^{NTR} isoforms. In some embodiments, the neurotrophic agent recognizes a TrkA receptor. In some embodiments, the neurotrophic agent recognizes a TrkB receptor. In some embodiments, the neurotrophic agent recognizes a TrkC receptor. In some embodiments, the neurotrophic agent is a pan-neurotrophin.

Described herein, in certain embodiments, are neurotrophic agents have an increased binding affinity to one or more Trk receptors relative to the binding affinity of an equivalent wild type neurotrophin. In some embodiments, the increase in binding affinity is about 20%, 30%, 40%, 50%, 60%, 70%, 80%, 85%, 90%, 95%, 96%, 97%, 98%, 99% or more relative to the binding affinity of an equivalent wild type neurotrophin. In some embodiments, the neurotrophic agent has a decreased binding affinity to p75^{NTR} or decreased activation of p75^{NTR} relative to the binding affinity of an equivalent wild type neurotrophin or pro-neurotrophin. In some embodiments, the decrease in binding affinity is about 30%, 40%, 50%, 60%, 70%, 80%, 85%, 90%, 95%, 96%, 97%, 98%, 99% or more relative to the binding affinity of an equivalent wild type neurotrophin.

Described herein, in certain embodiments, are pharmaceutical compositions comprising a neurotrophic agent and an excipient. In some embodiments, the pharmaceutical composition is formulated for intramuscular, intrathecal, intra-arterial, intravenous, intraocular, intravitreal, intraconjunctival, subcutaneous, cutaneous, intracerebral, intracerebroventricular, topical, or intracranial administration.

Described herein, in certain embodiments, are methods of treating a non-otic disease or condition, comprising: administering to a subject in need thereof a neurotrophic agent, wherein the non-otic disease or condition is associated with a reduced Trk receptor activity.

Described herein, in certain embodiments, are methods of treating a non-otic disease or condition, comprising: administering to a subject in need thereof a neurotrophic agent, wherein the non-otic disease or condition is treated by an upregulation of a Trk receptor activity.

Described herein, in certain embodiments, are methods of treating a non-otic disease or condition, comprising: administering to a subject in need thereof a neurotrophic agent, wherein the non-otic disease or condition is associated with an upregulation of p75^{NTR} activity. In some embodiments, the reduced Trk receptor activity is relative to the Trk receptor activity in a subject who does not have the non-otic disease or condition. In some embodiments, the non-otic disease or condition comprises a neurodegenerative disease or a symptomatic or pre-symptomatic condition with alterations to synapses. In some embodiments, the neurodegenerative disease comprises polyglutamine expansion disorder, fragile X syndrome, fragile XE mental retardation, Friedreich's ataxia, myotonic dystrophy, spinocerebellar ataxia type 8, spinocerebellar ataxia type 12, Alexander disease, Alper's disease, Alzheimer's disease, amyotrophic lateral sclerosis (ALS), ataxia telangiectasia, Batten disease (Spielmeyer-Vogt-Sjogren-Batten disease), Canavan disease, Cockayne syndrome, corticobasal degeneration, Creutzfeldt-Jakob disease, diabetic neuropathy, diabetic retinopathy, dementias, glaucoma, ischemia stroke, keratoconjunctivitis sicca, Krabbe disease, Lewy body dementia, multiple sclerosis, multiple system atrophy, Parkinson's disease, Pelizaeus-Merzbacher disease, Pick's disease, primary lateral sclerosis, progressive muscular atrophy, progressive bulbar palsy, pseudobulbar palsy, retinitis pigmentosa, Refsum's disease, Sandhoff disease, Schilder's disease, spinal cord injury (SCI), spinal muscular atrophy (SMA), Steele-Richardson-Olszewski disease, and Tabes dorsalis. In some embodiments, the polyglutamine repeat disease is Huntington's disease (HD), dentatorubropallidoluysian atrophy, Kennedy's disease (also referred to as spinobulbar muscular atrophy), or a spinocerebellar ataxia selected from the group consisting of type 1, type 2, type 3 (Machado-Joseph disease), type 6, type 7, and type 17). In some embodiments, the non-otic disease or condition comprises at least one of corneal epithelial and Goblet cell defect. In some embodiments, the non-otic disease or condition comprises corneal ulcer. In some embodiments, the non-otic disease or condition comprises pain associated with osteoarthritis, rheumatoid arthritis, or cancer. In some embodiments, the non-otic disease or condition comprises amyotrophic lateral sclerosis (ALS), spinal muscular atrophy (SMA), glaucoma, spinal cord injury (SCI), or retinitis pigmentosa. In some embodiments, the non-otic disease or condition comprises a psychiatric disorder. In some embodiments, the subject is a human.

Described herein, in certain embodiments, are methods of activating a Trk receptor, comprising: contacting a cell with a neurotrophic agent and facilitating binding between the neurotrophic agent and the Trk receptor, wherein binding of the neurotrophic agent to the Trk receptor induces activation of the Trk signaling pathway. In some embodiments, the Trk receptor is a TrkA receptor. In some embodiments, the Trk receptor is a TrkB receptor. In some embodiments, the Trk receptor is a TrkC receptor. In some embodiments, the neurotrophic agent binds to TrkA receptor, TrkB receptor, TrkC receptor, or a combination thereof. In some embodiments, the neurotrophic agent binds to p75^{NTR}. In some embodiments, neurotrophic agent binds to p75^{NTR} at a reduced affinity compared to binding to TrkA receptor, TrkB receptor, or TrkC receptor. In some embodiments, neurotrophic agent activates p75^{NTR} at a reduced efficacy compared to wild type or naturally occurring neurotrophic agents. In some embodiments, the reduced activation or reduced efficacy is a reduction of about 30%, 40%, 50%, 60%, 70%, 80%, 85%, 90%, 95%, 96%, 97%, 98%, 99%, or more, relative to the p75^{NTR} activation by an equivalent wild type neurotrophin. In some embodiments, the neurotrophic agent is further formulated as a pharmaceutical composition for intramuscular, intrathecal, intra-arterial, intravenous, intraocular, intravitreal, intraconjunctival, subcutaneous, cutaneous, intracerebral, intracerebroventricular, topical, or intracranial administration. In some embodiments, the method is an in vivo method.

Described herein, in certain embodiments, are recombinant vectors comprising a polynucleic acid polymer encoding a neurotrophic agent. In some embodiments, the recombinant vector is a viral vector. In some embodiments, the viral vector is a lentiviral vector, an adenovirus vector, an adeno-associated viral vector, or a herpes simplex virus vector. In some embodiments, the recombinant vector is a non-viral vector.

Described herein, in certain embodiments, are kits comprising a neurotrophic agent.

Described herein, in certain embodiments, are kits comprising a polynucleic acid polymer encoding a neurotrophic agent.

Described herein, in certain embodiments, are neurotrophic agents comprising modifications at amino acid residue positions equivalent to amino acid residues 15, 114, and 115 set forth in SEQ ID NO: 2, wherein the neurotrophic agent is selected from NGF, NT-4, NT-5, BDNF or PNT. In some embodiments, the neurotrophic agent further comprises a modification at an amino residue position equivalent to at least one of amino acid residue 11, 68, 87, and 103 set forth in SEQ ID NO: 2.

Described herein, in certain embodiments, are neurotrophic agents comprising modifications at amino residue positions equivalent to amino acid residues 15 and 103 set forth in SEQ ID NO: 2, wherein the neurotrophic agent is selected from NGF, NT-4, NT-5, BDNF or PNT.

Described herein, in certain embodiments, are neurotrophic agents comprising modifications at amino residue positions equivalent to amino acid residues 15, 51, and 103 set forth in SEQ ID NO: 2, wherein the neurotrophic agent is selected from NGF, NT-4, NT-5, BDNF or PNT.

Described herein, in certain embodiments, are methods of treating a non-otic condition in a subject in need thereof, comprising: administering to the subject in need thereof a non-otic composition comprising a therapeutically effective amount of a neurotrophic agent and a pharmaceutically acceptable carrier. In some embodiments, the non-otic disease or condition is treated by an upregulation of a Trk receptor activity. In some embodiments, the non-otic disease or condition is associated with an upregulation of a p75^{NTR} activity. In some embodiments, the reduced Trk receptor activity is relative to the Trk receptor activity in a subject who does not have the non-otic disease or condition. In some embodiments, the non-otic disease or condition comprises a neurodegenerative disease or a symptomatic or pre-symptomatic condition with alterations to synapses. In some embodiments, the neurodegenerative disease comprises polyglutamine expansion disorder, fragile X syndrome, fragile XE mental retardation, Friedreich's ataxia, myotonic dystrophy, spinocerebellar ataxia type 8, spinocerebellar ataxia type 12, Alexander disease, Alper's disease, Alzheimer's disease, amyotrophic lateral sclerosis (ALS), ataxia telangiectasia, Batten disease (Spielmeyer-Vogt-Sjogren-Batten disease), Canavan disease, Cockayne syndrome, corticobasal degeneration, Creutzfeldt-Jakob disease, diabetic neuropathy, diabetic retinopathy, dementias, glaucoma, ischemia stroke, keratoconjunctivitis sicca, Krabbe disease, Lewy body dementia, multiple sclerosis, multiple system atrophy, Parkinson's disease, Pelizaeus-Merzbacher disease, Pick's disease, primary lateral sclerosis, progressive muscular atrophy, progressive bulbar palsy, pseudobulbar palsy, retinitis pigmentosa, Refsum's disease, Sandhoff disease, Schilder's disease, spinal cord injury (SCI), spinal muscular atrophy (SMA), Steele-Richardson-Olszewski disease, and Tabes dorsalis. In some embodiments, the polyglutamine repeat disease is Huntington's disease (HD), dentatorubropallidoluysian atrophy, Kennedy's disease (also referred to as spinobulbar muscular atrophy), or a spinocerebellar ataxia selected from the group consisting of type 1, type 2, type 3 (Machado-Joseph disease), type 6, type 7, and type 17). In some embodiments, the non-otic disease or condition comprises at least one of corneal epithelial and Goblet cell defect. In some embodiments, the non-otic disease or condition comprises corneal ulcer. In some embodiments, the non-otic disease or condition comprises pain associated with osteoarthritis, rheumatoid arthritis, or cancer In some embodiments, the non-otic disease or condition comprises amyotrophic lateral sclerosis (ALS), spinal muscular atrophy (SMA), glaucoma, spinal cord injury (SCI), or retinitis pigmentosa. In some embodiments, the non-otic disease or condition comprises a psychiatric disorder. In some embodiments, the subject is a human.

Described herein, in certain embodiments, are methods of treating a non-otic condition in a subject in need thereof, comprising: administering to the subject in need thereof a non-otic composition comprising a therapeutically effective amount of a neurotrophic agent and a pharmaceutically acceptable carrier. In some embodiments, the neurotrophic agent is a non-natural neurotrophic agent. In some embodiments, the non-natural neurotrophic agent comprises a modification at amino acid residue position equivalent to amino acid residue 15 set forth in SEQ ID NO: 2, and optionally comprises a modification at one or more amino acid residue positions equivalent to amino acid residues 11, 51, 68, 87, 103, 114, or 115 set forth in SEQ ID NO: 2. In some embodiments, the non-natural neurotrophic agent comprises nerve growth factor (NGF), a pro-form of nerve growth factor (proNGF), neurotrophin-3 (NT-3), a pro-form of neurotrophin-3 (proNT-3), neurotrophin-4 (NT-4), a pro-form of neurotrophin-4 (proNT-4), neurotrophin-5 (NT-5), a pro-form of neurotrophin-5 (proNT-5), brain-derived neurotrophic factor (BDNF), a pro-form of brain-derived neurotrophic factor (proBDNF), a pan-neurotrophin (PNT), or PNT-1. In some embodiments, the non-natural neurotrophic agent is NT-3 and wherein NT-3 comprises a modification at amino acid residue position equivalent to amino acid residue 15 set forth in SEQ ID NO: 2, and optionally comprises a modification at one or more amino acid residue positions equivalent to amino acid residues 11, 51, 68, 87, 103, 114, or 115 set forth in SEQ ID NO: 2. In some embodiments, the non-natural neurotrophic agent comprises about 70%, 80%, 85%, 90%, 91%, 92%, 93%, 94%, 95%, 96%, 97%, 98%, or 99% sequence identity to SEQ ID NOs: 22-34.

### BRIEF DESCRIPTION OF THE DRAWINGS

Various aspects of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts intraocular pressure (IOP) upon cauterization. The right eyes of each rat (OD, closed circles) are cauterized, and the left eyes of each rat (OS, closed squares) are normal IOP control. The IOP is measured on the indicated days. The difference in IOP between the cauterized eye and the normal eye is significantly different at each of the times shown (p≤0.01).

FIG. 2A depicts effects of Trk receptor agonists in a glaucoma rat model.

FIG. 2B depicts effects of Trk receptor agonists in an optic nerve axotomy rat model.

FIG. 3A depicts fluorogold-labeled retinal ganglion cells (RGCs) in retinas in a glaucoma rat model.

FIG. 3B depicts fluorogold-labeled retinal ganglion cells (RGCs) in retinas in an optic nerve axotomy rat model.

FIGS. 4A-4D depict graphs of effects of wild-type nerve growth factor (NGF) treatment in wild-type or transgenic mice overexpressing amyloid precursor protein (APP). Wild-type and APP mice are treated with vehicle, 2 μg of NGF, or 20 μg of NGF. Mean latency (seconds) on the Y-axis as compared to days (X-axis) is measured in wild-type mice and APP mice treated with vehicle or 2 μg of NGF (FIG. 4A). Mean latency (seconds) on the Y-axis as compared to days (X-axis) is measured in wild-type mice and APP mice treated with vehicle or 20 μg of NGF (FIG. 4A). Short-term memory (trial 1) and long-term memory (trial 2) are measured in wild-type and APP mice treated with vehicle or 2 μg of NGF (FIG. 4C) following completion of Morris Water Maze. Short-term memory (trial 1) and long-term memory (trial 2) are measured in wild-type and APP mice treated with vehicle or 2 μg of NGF (FIG. 4D).

FIGS. 5A-5B depict graphs of effects of NGF-C mutein treatment in wild-type or transgenic mice overexpressing amyloid precursor protein (APP). NGF-C comprises mutations in K32/K34/E35 (K32A/K34A/E35A). Wild-type and APP mice are treated with vehicle or 5 of NGF-C. Mean latency (seconds) on the Y-axis as compared to days (X-axis) is measured in wild-type mice and APP mice treated with vehicle or 5 μg of NGF-C(FIG. 5A). Short-term memory (trial 1) and long-term memory (trial 2) are measured in wild-type and APP mice treated with vehicle or 5 μg of NGF-C(FIG. 5B) following completion of Morris Water Maze.

### DETAILED DESCRIPTION OF THE INVENTION

Neurotrophins are dimeric polypeptide growth factors that regulate the peripheral and central nervous systems and other tissues and promote functions such as neuronal survival and regulation of synaptic plasticity. In some instances, the family of neurotrophins includes nerve growth factor (NGF), brain-derived neurotrophic factor (BDNF), neurotrophin-3 (NT-3), neurotrophin-4 (NT-4), and neurotrophin-5 (NT-5). In some instances, neurotrophins mediate their effects through interaction with either the Trk family of receptors or with the p75 neurotrophin receptor which belongs to the tumor necrosis factor receptor superfamily. In some cases, interaction with the Trk family of receptors activates several signaling cascades, such as the phosphatidylinositol-3-kinase, phospholipase C-γ, and Ras/mitogen-activated protein kinase pathways, which mediate growth and survival responses of the neurotrophins. In some cases, interaction with the p75 family of receptors activates signaling cascades associated with neuronal death, synaptic loss, and production of neurotoxic cytokines.

In some embodiments, disclosed herein are compositions, formulations, methods of use, kits, and delivery devices for treating a non-otic indication with a neurotrophic agent. In some embodiments, the neurotrophic agent is a neurotrophin. In some instances, the neurotrophin is at least one of NGF, NT-3, NT-4, NT-5, and BDNF. In some embodiments, the neurotrophic agent comprises a neurotrophin with at least one modification. In some embodiments, the neurotrophic agent has an increased binding affinity for a Trk receptor. In some embodiments, the neurotrophic agent has a decreased binding affinity for p75^{NTR}, or is less efficient at activation of p75^{NTR}. In some embodiments, the neurotrophic agent is used to treat a non-otic disease or condition such as a neurodegenerative disease or disorder. In some embodiments, the non-otic disease or condition is characterized by at least one of reduced Trk receptor activity, upregulated Trk receptor activity, and upregulated p75^{NTR} activity.

Tropomyosin receptor kinase (Trk) agonists

Trk tyrosine kinase receptors are multi-domain single-transmembrane receptors that play an important role in a wide spectrum of neuronal responses including survival, differentiation, growth, and regeneration. Trk receptors are widely distributed in the central nervous system and the peripheral nervous system, and play a key role in neuronal survival, differentiation, and maintenance of proper function.

There are three members of the Trk family: TrkA, TrkB, and TrkC, encoded, respectively, by the genes Ntrk1, Ntrk2, and Ntrk3 in rat or mouse genomic nomenclature, and by NTRK1, NTRK2, and NTRK3 in human genomic nomenclature. The extracellular domains of native TrkA, TrkB, and TrkC receptors have five functional domains that have been defined with reference to homologous or otherwise similar structures identified in various other proteins. The domains have been designated starting at the N-terminus of the amino acid sequence of the mature Trk receptors as 1) a first cysteine-rich domain extending from amino acid position 1 to about amino acid position 32 of human TrkA, from amino acid position 1 to about amino acid position 36 of human TrkB, and from amino acid position 1 to about amino acid position 48 of human TrkC; 2) a leucine-rich domain stretching from about amino acid 33 to about amino acid to about amino acid 104 in TrkA; from about amino acid 37 to about amino acid 108 in TrkB, and from about amino acid 49 to about amino acid 120 in TrkC; 3) a second cysteine-rich domain from about amino acid 105 to about amino acid 157 in TrkA: from about amino acid 109 to about amino acid 164 in TrkB; and from about amino acid 121 to about amino acid 177 in TrkC; 4) a first immunoglobulin-like domain stretching from about amino acid 176 to about amino acid 234 in TrkA; from about amino acid 183 to about amino acid 239 in TrkB; and from about amino acid 196 to about amino acid 257 in TrkC; and 5) a second immunoglobulin-like domain extending from about amino acid 264 to about amino add 330 in TrkA; from about amino acid 270 to about amino acid 334 in TrkB; and from about amino acid 288 to about amino acid 351 in TrkC.

In some instances, the tropomyosin receptor kinases are characterized as high affinity receptors for naturally occurring neurotrophins, a family of protein growth factors which includes nerve growth factor (NGF), brain derived neurotrophic factor (BDNF), neurotrophin-3 (NT-3), and neurotrophins-4/5 (NT-4/5). In some cases, NT-3, BDNF, and NGF are referred to as essential growth factors for the development and maintenance of the nervous system.

In some embodiments, Trk receptor ectodomain termed D5 comprises the main neurotrophin binding site and are required for ligand-dependent receptor activation. In some instances, the Trk receptor ectodomain D4 further mediates ligand-dependent receptor activation. In additional instances, the Trk receptor ectodomain D1 is involved in mediating ligand-dependent receptor activation. Such receptor sites that define ligand-binding and functional-activation are termed “hot spots”.

Mature neurotrophins bind a selective Trk receptor with relatively high affinity (e.g. TrkB-BDNF, TrkA-NGF, and TrkC-NT-3). In some cases, TrkC is the preferred receptor for NT-3 and mediates the multiple effects of NT-3, including neuronal death or survival and cellular differentiation. The Trk receptor has tyrosine kinase catalytic activity that is associated with the survival and differentiation of neurotrophic signals. Neurotrophin-induced Trk activity affords trophic (growth/survival) responses via MAPK and AKT, whereas PLC-γ and fibroblast growth factor receptor substrate-2 (FRS-2) activity are involved in differentiation.

In some embodiments, the mature neurotrophins (e.g., NGF, NT-3, NT-4, NT-5, and BDNF) also bind to p75^{NTR}, a neurotrophin receptor which binds all neurotrophins with lower affinity but, in complex with the ubiquitous protein sortilin, makes a high-affinity receptor for precursor of mature neurotrophins or proneurotrophins. p75^{NTR} is not a receptor protein-tyrosine kinase and recruits intracellular signaling different from that activated by Trks. p75^{NTR} signaling is generally atrophic, promoting apoptosis, inhibiting neurite growth, promoting inflammatory cascades, and depressing synaptic strength. The p75^{NTR} is expressed on glial cells as well as on neurons. In the peripheral nervous system, p75^{NTR} is expressed on Schwann cells after axotomy. When p75^{NTR} is expressed in non-neuronal cells, its activation can cause pro-inflammatory responses. When p75^{NTR} is expressed in neuronal cells together with Trks, the p75^{NTR} receptor can affect Trk-ligand binding or Trk function, although the mechanism is not fully understood. It has been shown that p75^{NTR} can unmask a cryptic “hot spot” of Trk receptors, suggesting the notion of allosteric regulation.

Described herein, in some embodiments, are non-otic compositions comprising agonists for Trk receptors. In some embodiments, the agonists for Trk receptors comprise a neurotrophic agent. In some instances, the neurotrophic agent is a mutant of at least one of NGF, BDNF, NT-3, NT-4, and NT-5. In some cases, the neurotrophic agent comprises a proneurotrophin form. In other cases, the neurotrophic agent comprises a mature neurotrophin form.

### Neurotrophic Agents

Described herein, in some embodiments, are non-otic compositions comprising a Trk receptor agonist, wherein the agonist is a neurotrophic agent. In some embodiments, the Trk receptor agonist is a neurotrophic agent that selectively binds to TrkA receptor. In some embodiments, the Trk receptor agonist is a neurotrophic agent that selectively binds to TrkB receptor. In some embodiments, the Trk receptor agonist is a neurotrophic agent that selectively binds to TrkC receptor. In some embodiments, the neurotrophic agent binds one or more Trk receptors. In some embodiments, the Trk receptor agonist is a neurotrophic agent that does not bind to the neurotrophic receptor p75^{NTR}, or activates the p75^{NTR} with lower efficacy than naturally occurring neurotrophins. In some embodiments, the Trk receptor agonist is a neurotrophic agent that binds to the neurotrophic receptor p75^{NTR} at a reduced affinity. In some embodiments, the neurotrophic agent recognizes an isoform of at least one of TrkA, TrkB, TrkC, and p75^{NTR}.

In some embodiments, a neurotrophic agent is an agent that promotes growth and survival of neurons and non-neuronal cells such as glial cells. In some embodiments, the neurotrophic agent is an agent that mediates neuronal and non-neuronal cell development. In some embodiments, the neurotrophic agent is an agent that mediates synapse activity. In some embodiments, the neurotrophic agent is an agent that mediates at least one of differentiation and proliferation. In some embodiments, the neurotrophic agent is a neurotrophin. In some embodiments, the neurotrophin is nerve growth factor (NGF), brain-derived neurotrophic factor (BDNF), ciliary neurotrophic factor (CNTF), glial cell-line derived neurotrophic factor (GDNF), neurotrophin-3 (NT-3), neurotrophin-4 (NT-4), neurotrophin-5 (NT-5), a pan-neurotrophin, or a chimeric neurotrophin. In some embodiments, the neurotrophin is a pro-form, or precursor, of NGF, BDNF, NT-3, NT-4, or NT-5. In some embodiments, the neurotrophin is an isoform of NGF, BDNF, NT-3, NT-4, or NT-5. In some embodiments, the neurotrophin is NGF. In some embodiments, the neurotrophic agent is BDNF. In some embodiments, the neurotrophin is NT-3. In some embodiments, the neurotrophin is NT-4. In some embodiments, the neurotrophin is NT-5. In some embodiments, the neurotrophin is a pan-neurotrophin. In some embodiments, the neurotrophin is a chimeric neurotrophin. Exemplary sequences of neurotrophins described herein are illustrated in Table 1.

TABLE 1

| Protein | | SEQ ID NO: |
|---|---|---|
| Nerve Growth | SSSHPIFHRGEFSVCDSVSVWVGDKTTATDIKGKEVMVLG | 1 |
| Factor | EVNINNSVFKQYFFETKCRDPNPVDSGCRGIDSKHWNSYC | |
| (mature form) | TTTHTFVKALTMDGKQAAWRFIRIDTACVCVLSRKAVRR | |
| | A | |
| Neurotrophin 3 | YAEHKSHRGEYSVCDSESLWVTDKSSAIDIRGHQVTVLGEI | 2 |
| (mature form) | KTGNSPVKQYFYETRCKEARPVKNGCRGIDDKHWNSQCK | |
| | TSQTYVRALTSENNKLVGWRWIRIDTSCVCALSRKIGRT | |
| Neurotrophin 4/5 | GVSETAPASRRGELAVCDAVSGWVTDRRTAVDLRGREVE | |
| (mature form) | VLGEVPAAGGSPLRQYFFETRCKADNAEEGGPGAGGGGC | 3 |
| | RGVDRRHWVSECKAKQSYVRALTADAQGRVGWRWIRID | |
| | TACVCTLLSRTGRA | |
| Brain-derived | HSDPARRGELSVCDSISEWVTAADKKTAVDMSGGTVTVL | 4 |
| neurotrophic | EKVPVSKGQLKQYFYETKCNPMGYTKEGCRGIDKRHWNS | |
| factor | QCRTTQSYVRALTMDSKKRIGWRFIRIDTSCVCTLTIKRGR | |
| (mature form) | | |
| PNT-1 | SSSHPIFHRGEYSVCDSESLWVTDKSSAIDIRGHQVTVLGEI | 5 |
| | KTGNSPVKQYFYETRCKEARPVKNGCRGIDDKHWNSQCK | |
| | TSQTYVRALTSESKKRIGWRWIRIDTSCVCALSRKIGRT | |
| NGF/BDNF | SSSHPIFHRGEFSVCDSVSVWVGDKTTATDIKGKEVMVLG | 6 |
| chimera 1 (3 + 4 + 5) | EVNINNSVFKQYFFETKCNPMGYTKEGCRGIDSKHWNSQC | |
| | RTTQSYVRALTMDSKKRIGWRFIRIDTACVCVLSRKAVRR | |
| | A | |
| NGF/BDNF | SSSHPIFHRGEFSVCDSVSVWVTAADKKTAVDMSGGTVM | 7 |
| chimera 2 | VLGEVNINNSVFKQYFFETKCNPMGYTKEGCRGIDSKHWN | |
| (1 + 3 + 4 + 5) | SQCRTTQSYVRALTMDSKKRIGWRFIRIDTACVCVLSRKA | |
| | VRRA | |

Described herein, in some embodiments, are non-otic compositions comprising a Trk receptor agonist, wherein the agonist is a neurotrophic agent. In some embodiments, the neurotrophic agent comprises NGF, NT-3, NT-4, NT-5, BDNF, a pan-neurotrophin, or a chimeric neurotrophin. In some embodiments, the neurotrophic agent comprises at most 1 amino acid modification, at most 2 amino acid modifications, at most 3 amino acid modifications, at most 4 amino acid modifications, at most 5 amino acid modifications, at most 6 amino acid modifications, at most 7 amino acid modifications, at most 8 amino acid modifications, at most 9 amino acid modifications, at most 10 amino acid modifications, or another suitable number of modifications as compared to a neurotrophic agent comprising a sequence set forth in SEQ ID NO: 1, 2, 3, 4, 5, 6, or 7. In some embodiments, the neurotrophic agent comprises at least 1 amino acid modification, at least 2 amino acid modifications, at least 3 amino acid modifications, at least 4 amino acid modifications, at least 5 amino acid modifications, at least 6 amino acid modifications, at least 7 amino acid modifications, at least 8 amino acid modifications, at least 9 amino acid modifications, at least 10 amino acid modifications, or another suitable number of modifications as compared to a neurotrophic agent comprising a sequence set forth in SEQ ID NO: 1, 2, 3, 4, 5, 6, or 7.

In some embodiments, a neurotrophic agent described herein comprises NGF. In some embodiments, the neurotrophic agent comprises a pro-form of NGF. In some instances, the neurotrophic agent comprises a mature form of NGF. In additional instances, the neurotrophic agent comprises an isoform of NGF. In some embodiments, the neurotrophic agent comprises the sequence set forth in SEQ ID NO: 1. In some embodiments, the neurotrophic agent further comprises at most 1 amino acid modification, at most 2 amino acid modifications, at most 3 amino acid modifications, at most 4 amino acid modifications, at most 5 amino acid modifications, at most 6 amino acid modifications, at most 7 amino acid modifications, at most 8 amino acid modifications, at most 9 amino acid modifications, at most 10 amino acid modifications, or another suitable number of modifications. In some embodiments, the neurotrophic agent further comprises at least 1 amino acid modification, at least 2 amino acid modifications, at least 3 amino acid modifications, at least 4 amino acid modifications, at least 5 amino acid modifications, at least 6 amino acid modifications, at least 7 amino acid modifications, at least 8 amino acid modifications, at least 9 amino acid modifications, at least 10 amino acid modifications, or another suitable number of modifications. In some instances, the neurotrophic agent consists of the sequence set forth in SEQ ID NO: 1. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a neurotrophic agent described herein is neurotrophin-3 (NT-3). In some embodiments, neurotrophin-3 promotes the survival of existing neurons and their processes and connections and promotes the differentiation of neural cell progenitors. Further, in some embodiments, neurotrophin-3 protects the Cranial Nerve VIII from degeneration. In some embodiments, the neurotrophic agent comprises a pro-form of NT-3. In some instances, the neurotrophic agent comprises a mature form of NT-3. In other instances, the neurotrophic agent comprises an isoform of NT-3. In some embodiments, the neurotrophic agent comprises the sequence set forth in SEQ ID NO: 2. In some embodiments, the neurotrophic agent further comprises at most 1 amino acid modification, at most 2 amino acid modifications, at most 3 amino acid modifications, at most 4 amino acid modifications, at most 5 amino acid modifications, at most 6 amino acid modifications, at most 7 amino acid modifications, at most 8 amino acid modifications, at most 9 amino acid modifications, at most 10 amino acid modifications, or another suitable number of modifications. In some embodiments, the neurotrophic agent further comprises at least 1 amino acid modification, at least 2 amino acid modifications, at least 3 amino acid modifications, at least 4 amino acid modifications, at least 5 amino acid modifications, at least 6 amino acid modifications, at least 7 amino acid modifications, at least 8 amino acid modifications, at least 9 amino acid modifications, at least 10 amino acid modifications, or another suitable number of modifications. In some cases, the neurotrophic agent consists of the sequence set forth in SEQ ID NO: 2. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, the neurotrophic agent comprises a naturally occurring neurotrophin-3 with one or more mutations which comprises NT-3₍₁₋₁₁₉₎ or NT-3₍₁₋₁₁₇₎ as described in PCT Pub. No. WO9803546.

In some embodiments, the neurotrophic agent comprises a naturally occurring neurotrophin-3 with one or more mutations that comprises a NT-3 mutant described in Urfer, et al., “The binding epitopes of neurotrophin-3 to its receptors TrkC and gp75 and the design of a multifunctional human neurotrophin,” EMBO 13(24): 5896-5909 (1994).

In some embodiments, a neurotrophic agent described herein is neurotrophin-4 (NT-4). In some embodiments, the neurotrophic agent comprises a pro-form of NT-4. In some instances, the neurotrophic agent comprises a mature form of NT-4. In other instances, the neurotrophic agent comprises an isoform of NT-4. In some embodiments, the neurotrophic agent comprises the sequence set forth in SEQ ID NO: 3. In some embodiments, the neurotrophic agent further comprises at most 1 amino acid modification, at most 2 amino acid modifications, at most 3 amino acid modifications, at most 4 amino acid modifications, at most 5 amino acid modifications, at most 6 amino acid modifications, at most 7 amino acid modifications, at most 8 amino acid modifications, at most 9 amino acid modifications, at most 10 amino acid modifications, or another suitable number of modifications. In some embodiments, the neurotrophic agent further comprises at least 1 amino acid modification, at least 2 amino acid modifications, at least 3 amino acid modifications, at least 4 amino acid modifications, at least 5 amino acid modifications, at least 6 amino acid modifications, at least 7 amino acid modifications, at least 8 amino acid modifications, at least 9 amino acid modifications, at least 10 amino acid modifications, or another suitable number of modifications. In some cases, the neurotrophic agent consists of the sequence set forth in SEQ ID NO: 3. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a neurotrophic agent described herein is neurotrophin-5 (NT-5). In some embodiments, the neurotrophic agent comprises a pro-form of NT-5. In some instances, the neurotrophic agent comprises a mature form of NT-5. In other instances, the neurotrophic agent comprises an isoform of NT-5. In some embodiments, the neurotrophic agent comprises of the sequence set forth in SEQ ID NO: 3. In some embodiments, the neurotrophic agent further comprises at most 1 amino acid modification, at most 2 amino acid modifications, at most 3 amino acid modifications, at most 4 amino acid modifications, at most 5 amino acid modifications, at most 6 amino acid modifications, at most 7 amino acid modifications, at most 8 amino acid modifications, at most 9 amino acid modifications, at most 10 amino acid modifications, or another suitable number of modifications. In some embodiments, the neurotrophic agent further comprises at least 1 amino acid modification, at least 2 amino acid modifications, at least 3 amino acid modifications, at least 4 amino acid modifications, at least 5 amino acid modifications, at least 6 amino acid modifications, at least 7 amino acid modifications, at least 8 amino acid modifications, at least 9 amino acid modifications, at least 10 amino acid modifications, or another suitable number of modifications. In some cases, the neurotrophic agent consists of the sequence set forth in SEQ ID NO: 3. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a neurotrophic agent described herein is BDNF. In some embodiments, the neurotrophic agent comprises a pro-form of BDNF. In some instances, the neurotrophic agent comprises a mature form of BDNF. In other instances, the neurotrophic agent comprises an isoform of BDNF. In some embodiments, the neurotrophic agent comprises the sequence set forth in SEQ ID NO: 4. In some embodiments, the neurotrophic agent further comprises at most 1 amino acid modification, at most 2 amino acid modifications, at most 3 amino acid modifications, at most 4 amino acid modifications, at most 5 amino acid modifications, at most 6 amino acid modifications, at most 7 amino acid modifications, at most 8 amino acid modifications, at most 9 amino acid modifications, at most 10 amino acid modifications, or another suitable number of modifications. In some embodiments, the neurotrophic agent further comprises at least 1 amino acid modification, at least 2 amino acid modifications, at least 3 amino acid modifications, at least 4 amino acid modifications, at least 5 amino acid modifications, at least 6 amino acid modifications, at least 7 amino acid modifications, at least 8 amino acid modifications, at least 9 amino acid modifications, at least 10 amino acid modifications, or another suitable number of modifications. In some cases, the neurotrophic agent consists of the sequence set forth in SEQ ID NO: 4. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a neurotrophic agent described herein is a pan-neurotrophin (PNT). In some instances, a pan-neurotrophin is a synthetic trophic factor engineered by combining one or more domains of nerve growth factor (NGF), brain-derived neurotrophic factor (BDNF), and/or neurotrophin 3 (NT-3). In some instances, a pan-neurotrophin recognizes or binds to TrkA, TrkB, and TrkC receptors. In some instances, a pan-neurotrophin is pan-neurotrophin 1 (PNT-1), described in Ilag, et al., “Pan-neurotrophin 1: A genetically engineered neurotrophic factor displaying multiple specificities in peripheral neurons in vitro and in vivo,” PNAS 92: 607-611 (1995). In some cases, a pan-neurotrophin is a pan-neurotrophin described in Ibanez, et al, “An extended surface of binding to Trk tyrosine kinase receptors in NGF and BDNF allows the engineering of a multifunctional pan-neurotrophin,” EMBO 12(6): 2281-2293 (1993).

In some embodiments, the neurotrophic agent comprises PNT. In some embodiments, the neurotrophic agent comprises the sequence set forth in SEQ ID NO: 5. In some embodiments, the neurotrophic agent further comprises at most 1 amino acid modification, at most 2 amino acid modifications, at most 3 amino acid modifications, at most 4 amino acid modifications, at most 5 amino acid modifications, at most 6 amino acid modifications, at most 7 amino acid modifications, at most 8 amino acid modifications, at most 9 amino acid modifications, at most 10 amino acid modifications, or another suitable number of modifications. In some embodiments, the neurotrophic agent further comprises at least 1 amino acid modification, at least 2 amino acid modifications, at least 3 amino acid modifications, at least 4 amino acid modifications, at least 5 amino acid modifications, at least 6 amino acid modifications, at least 7 amino acid modifications, at least 8 amino acid modifications, at least 9 amino acid modifications, at least 10 amino acid modifications, or another suitable number of modifications. In some cases, the neurotrophic agent consists of the sequence set forth in SEQ ID NO: 5 (PNT-1). In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some instances, a neurotrophic agent described herein is a chimeric neurotrophin. In some instances, the chimeric neurotrophin recognizes two or more Trk receptors. In some embodiments, the neurotrophic agent is a chimera of NGF and BDNF. In some cases, a chimeric neurotrophic agent comprises, for example, one or more domains of nerve growth factor (NGF) and one or more domains of brain-derived neurotrophic factor (BDNF). In some instances, the neurotrophic agent is a chimera of NGF and BDNF described in Ibanez, et al., “Chimeric molecules with multiple neurotrophic activities reveal structural elements determining the specificities of NGF and BDNF,” EMBO 10(8): 2105-2110, 1991; Ryden, et al., “Functional analysis of mutant neurotrophins deficient in low-affinity binding reveals a role for p75LNGFR in NT-4 signalling,” EMBO 14(9): 1979-1990, 1995; and/or Ibanez, et al., “An extended surface of binding to Trk tyrosine kinase receptors in NGF and BDNF allows the engineering of a multifunctional pan-neurotrophin,” EMBO 12(6): 2281-2293, 1993.

In some embodiments, the chimeric neurotrophin binds to two or more Trk receptors. In some embodiments, the chimeric neurotrophin binds two or more Trk receptors and further has a reduced binding affinity to p75^{NTR}, or activates the p75^{NTR} with lower efficacy than naturally occurring neurotrophins. In some embodiments, the chimeric neurotrophin, binds to two or more Trk receptors but does not bind to p75^{NTR}. In some embodiments, the chimeric neurotrphin is characterized with an improved neural or non-neural survival, differentiation, growth, regeneration compared to a non-chimeric neurotrophin or a combination thereof, relative to a non-chimeric neurotrophin. In some embodiments, the chimeric neurotrophin has an increased binding affinity, efficacy, potency, or a combination thereof, relative to a non-chimeric neurotrophin.

In some embodiments, the chimera of NGF and BDNF comprises the amino acid sequence set forth in SEQ ID NO: 6 or 7. In some embodiments, the neurotrophic agent further comprises at most 1 amino acid modification, at most 2 amino acid modifications, at most 3 amino acid modifications, at most 4 amino acid modifications, at most 5 amino acid modifications, at most 6 amino acid modifications, at most 7 amino acid modifications, at most 8 amino acid modifications, at most 9 amino acid modifications, at most 10 amino acid modifications, or another suitable number of modifications. In some embodiments, the neurotrophic agent further comprises at least 1 amino acid modification, at least 2 amino acid modifications, at least 3 amino acid modifications, at least 4 amino acid modifications, at least 5 amino acid modifications, at least 6 amino acid modifications, at least 7 amino acid modifications, at least 8 amino acid modifications, at least 9 amino acid modifications, at least 10 amino acid modifications, or another suitable number of modifications. In some instances, the neurotrophic agent consists of the sequence set forth in SEQ ID NO: 6. In some instances, the neurotrophic agent consists of the sequence set forth in SEQ ID NO: 7. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a neurotrophic agent described herein comprises a modification at an amino acid residue position equivalent to at least one of an amino acid residue 7, 84, 103, 45, 32, 34, and 35 set forth in SEQ ID NO: 1. In some embodiments, the neurotrophic agent comprises modifications at amino acid residue positions equivalent to amino acid residues 7 and 103 set forth in SEQ ID NO: 1. In some cases, the neurotrophic agent further comprises a modification at an amino acid residue position equivalent to amino acid residue 45 or 84 set forth in SEQ ID NO: 1. In some cases, the neurotrophic agent further comprises a modification at an amino acid residue position equivalent to amino acid residue 84 set forth in SEQ ID NO: 1. In some embodiments, the neurotrophic agent further comprises a modification at an amino acid residue position equivalent to amino acid residue 45 set forth in SEQ ID NO: 1. In some embodiments, the neurotrophic agent further comprises modifications at amino acid residue positions equivalent to amino acid residues 32, 34, and 35 set forth in SEQ ID NO: 1. In some embodiments, the neurotrophic agent comprises modifications at amino acid residue positions equivalent to amino acid residues 7, 84, and 103 set forth in SEQ ID NO: 1. In some embodiments, the neurotrophic agent comprises modifications at amino acid residue positions equivalent to amino acid residues 7, 45, and 103 set forth in SEQ ID NO: 1. In some embodiments, the neurotrophic agent comprises modifications at amino acid residue positions equivalent to amino acid residues 7, 32, 34, 35, 45, and 103 set forth in SEQ ID NO: 1. In some embodiments, the neurotrophic agent comprises modifications at amino acid residue positions equivalent to amino acid residues 7, 32, 34, 35, 84, and 103 set forth in SEQ ID NO: 1. In some instances, the neurotrophic agent is NGF, NT-3, NT-4, NT-5, BDNF, a pan-neurotrophic agent, or a chimeric neurotrophin. In some instances, the chimeric neurotrophin comprises a sequence set forth in SEQ ID NO: 6 or 7. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a neurotrophic agent is NGF. In some embodiments, NGF comprises a modification at an amino acid residue position equivalent to at least one of an amino acid residue 7, 84, 103, 45, 32, 34, and 35 set forth in SEQ ID NO: 1. In some embodiments, NGF comprises modifications at amino acid residue positions equivalent to amino acid residues 7 and 103 set forth in SEQ ID NO: 1. In some cases, NGF further comprises a modification at an amino acid residue position equivalent to amino acid residue 45 or 84 set forth in SEQ ID NO: 1. In some cases, NGF further comprises a modification at an amino acid residue position equivalent to amino acid residue 84 set forth in SEQ ID NO: 1. In some embodiments, NGF further comprises a modification at an amino acid residue position equivalent to amino acid residue 45 set forth in SEQ ID NO: 1. In some embodiments, NGF further comprises modifications at amino acid residue positions equivalent to amino acid residues 32, 34, and 35 set forth in SEQ ID NO: 1. In some embodiments, NGF comprises modifications at amino acid residue positions equivalent to amino acid residues 7, 84, and 103 set forth in SEQ ID NO: 1. In some embodiments, NGF comprises modifications at amino acid residue positions equivalent to amino acid residues 7, 45, and 103 set forth in SEQ ID NO: 1. In some embodiments, NGF comprises modifications at amino acid residue positions equivalent to amino acid residues 7, 32, 34, 35, 45, and 103 set forth in SEQ ID NO: 1. In some embodiments, NGF comprises modifications at amino acid residue positions equivalent to amino acid residues 7, 32, 34, 35, 84, and 103 set forth in SEQ ID NO: 1. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a neurotrophic agent comprises the mature form of NGF. In some embodiments, the neurotrophic agent has a modification to at least one of an amino acid residue 7, 84, 103, 45, 32, 34, and 35 set forth in SEQ ID NO: 1. In some embodiments, the neurotrophic agent comprises modifications at amino acid residues 7 and 103 set forth in SEQ ID NO: 1. In some cases, the neurotrophic agent further comprises a modification at amino acid residue 45 or 84 set forth in SEQ ID NO: 1. In some cases, the neurotrophic agent further comprises a modification at amino acid residue 84 set forth in SEQ ID NO: 1. In some embodiments, the neurotrophic agent further comprises a modification at amino acid residue 45 set forth in SEQ ID NO: 1. In some embodiments, the neurotrophic agent further comprises modifications at amino acid residues 32, 34, and 35 set forth in SEQ ID NO: 1. In some embodiments, the neurotrophic agent comprises modifications at amino acid residues 7, 84, and 103 set forth in SEQ ID NO: 1. In some embodiments, the neurotrophic agent comprises modifications at amino acid residues 7, 45, and 103 set forth in SEQ ID NO: 1. In some embodiments, the neurotrophic agent comprises modifications at amino acid residues 7, 32, 34, 35, 45, and 103 set forth in SEQ ID NO: 1. In some embodiments, the neurotrophic agent comprises modifications at amino acid residues 7, 32, 34, 35, 84, and 103 set forth in SEQ ID NO: 1. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a neurotrophic agent described herein comprises a modification at an amino acid residue position equivalent to at least one of an amino acid residue 32, 34, 61, 74, 75, 77, 100, 115, 116, and 118 set forth in SEQ ID NO: 5. In some embodiments, the neurotrophic agent comprises at least two modifications selected from amino acid residue positions equivalent to amino acid residues 74, 75, and 77 set forth in SEQ ID NO: 5. In some embodiments, the neurotrophic agent further comprises a modification at an amino acid residue position equivalent to amino acid residue 32, 34, 115, or 116 set forth in SEQ ID NO: 5. In some embodiments, the neurotrophic agent further comprises modifications at amino acid residue positions equivalent to amino acid residues 32, 34, 115, and 116 set forth in SEQ ID NO: 5. In some embodiments, the neurotrophic agent comprises modifications at amino acid residue positions equivalent to amino acid residues 32, 34, 74, 75, 77, 115, and 116 set forth in SEQ ID NO: 5. In some instances, the neurotrophic agent is NGF, NT-3, NT-4, NT-5, BDNF, a pan-neurotrophic agent, or a chimeric neurotrophin. In some instances, the chimeric neurotrophin comprises a sequence set forth in SEQ ID NO: 6 or 7. In some instances, the modification is a mutation. For example, the mutation is a mutation to at least one of a non-polar residue, a polar residue, and a charged residue. In some instances, the mutation is to alanine. In some cases, the mutation is a conservative mutation, a semi-conservative mutation, or a non-conservative mutation.

In some embodiments, a neurotrophic agent is PNT. In some embodiments, PNT comprises a modification at an amino acid residue position equivalent to at least one of an amino acid residue 32, 34, 61, 74, 75, 77, 100, 115, 116, and 118 set forth in SEQ ID NO: 5. In some embodiments, PNT comprises at least two modifications selected from amino acid residue positions equivalent to amino acid residues 74, 75, and 77 set forth in SEQ ID NO: 5. In some embodiments, PNT further comprises a modification at an amino acid residue position equivalent to amino acid residue 32, 34, 115, or 116 set forth in SEQ ID NO: 5. In some embodiments, PNT further comprises modifications at amino acid residue positions equivalent to amino acid residues 32, 34, 115, and 116 set forth in SEQ ID NO: 5. In some embodiments, PNT comprises modifications at amino acid residue positions equivalent to amino acid residue 32, 34, 74, 75, 77, 115, and 116 set forth in SEQ ID NO: 5. In some instances, the modification is a mutation. For example, the mutation is a mutation to at least one of a non-polar residue, a polar residue, and a charged residue. In some instances, the mutation is to alanine. In some cases, the mutation is a conservative mutation, a semi-conservative mutation, or a non-conservative mutation.

In some embodiments, a neurotrophic agent comprises PNT-1. In some embodiments, the neurotrophic agent comprises a modification at least one of an amino acid residue 32, 34, 61, 74, 75, 77, 100, 115, 116, and 118 set forth in SEQ ID NO: 5. In some embodiments, the neurotrophic agent comprises at least two modifications selected from amino acid residues 74, 75, and 77 set forth in SEQ ID NO: 5. In some embodiments, the neurotrophic agent further comprises a modification at amino acid residue 32, 34, 115, or 116 set forth in SEQ ID NO: 5. In some embodiments, the neurotrophic agent further comprises modifications at amino acid residue 32, 34, 115, or 116 set forth in SEQ ID NO: 5. In some embodiments, the neurotrophic agent comprises modifications at amino acid residue 32, 34, 74, 75, 77, 115, and 116 set forth in SEQ ID NO: 5. In some instances, the modification is a mutation. For example, the mutation is a mutation to at least one of a non-polar residue, a polar residue, and a charged residue. In some instances, the mutation is to alanine. In some cases, the mutation is a conservative mutation, a semi-conservative mutation, or a non-conservative mutation.

In some embodiments, a neurotrophic agent comprises modifications at amino acid residue positions equivalent to amino acid residues 32 and 34 set forth in SEQ ID NO: 5. In some embodiments, the neurotrophic agent further comprises a modification at an amino acid residue position equivalent to amino acid residue 74, 75, 77, 115, or 116 set forth in SEQ ID NO: 5. In some embodiments, the neurotrophic agent further comprises modifications at amino acid residue positions equivalent to amino acid residues 74, 75, 77, 115, and 116 set forth in SEQ ID NO: 5. In some embodiments, the neurotrophic agent is selected from NGF, NT-4, NT-5, BDNF, PNT, or a chimeric neurotrophin. In some instances, the chimeric neurotrophin comprises a sequence set forth in SEQ ID NO: 6 or 7. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a neurotrophic agent is PNT. In some embodiments, PNT comprises modifications at amino acid residue positions equivalent to amino acid residues 32 and 34 set forth in SEQ ID NO: 5. In some embodiments, PNT further comprises a modification at an amino acid residue position equivalent to amino acid residue 74, 75, 77, 115 or 116 set forth in SEQ ID NO: 5. In some embodiments, PNT further comprises modifications at amino acid residue positions equivalent to amino acid residues 74, 75, 77, 115 and 116 set forth in SEQ ID NO: 5. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a neurotrophic agent comprises PNT-1. In some embodiments, the neurotrophic agent comprises modifications at amino acid residues 32 and 34 set forth in SEQ ID NO: 5. In some embodiments, the neurotrophic agent further comprises a modification at amino acid residue 74, 75, 77, 115, or 116 set forth in SEQ ID NO: 5. In some embodiments, the neurotrophic agent further comprises modifications at amino acid residues 74, 75, 77, 115, and 116 set forth in SEQ ID NO: 5. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a neurotrophic agent comprises modifications at amino acid residue positions equivalent to amino acid residues 115 and 116 set forth in SEQ ID NO: 5. In some embodiments, the neurotrophic agent further comprises a modification at an amino acid residue position equivalent to amino acid residue 32, 34, 74, 75, or 77 set forth in SEQ ID NO: 5. In some embodiments, the neurotrophic agent further comprises modifications at amino acid residue positions equivalent to amino acid residues 32, 34, 74, 75, and 77 set forth in SEQ ID NO: 5. In some embodiments, the neurotrophic agent is selected from NGF, NT-4, NT-5, BDNF, PNT, or a chimeric neurotrophin. In some instances, the chimeric neurotrophin comprises a sequence set forth in SEQ ID NO: 6 or 7. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a neurotrophic agent is PNT. In some embodiments, PNT comprises modifications at amino acid residue positions equivalent to amino acid residues 115 and 116 set forth in SEQ ID NO: 5. In some embodiments, PNT further comprises a modification at an amino acid residue position equivalent to amino acid residue 32, 34, 74, 75, or 77 set forth in SEQ ID NO: 5. In some embodiments, PNT further comprises modifications at amino acid residue positions equivalent to amino acid residues 32, 34, 74, 75, and 77 set forth in SEQ ID NO: 5. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a neurotrophic agent comprises PNT-1. In some embodiments, the neurotrophic agent comprises modifications at amino acid residues 115 and 116 set forth in SEQ ID NO: 5. In some embodiments, the neurotrophic agent further comprises a modification at amino acid residue 32, 34, 74, 75, or 77 set forth in SEQ ID NO: 5. In some embodiments, the neurotrophic agent further comprises modifications at amino acid residues 32, 34, 74, 75, and 77 set forth in SEQ ID NO: 5. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a neurotrophic agent comprises modifications at amino acid residue positions equivalent to amino acid residues 61 and 100 set forth in SEQ ID NO: 5. In some embodiments, the neurotrophic agent is selected from NT-3, NT-4, NT-5, BDNF, PNT, or a chimeric neurotrophin. In some instances, the chimeric neurotrophin comprises a sequence set forth in SEQ ID NO: 6 or 7. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a neurotrophic agent is PNT. In some embodiments, PNT comprises modifications at amino acid residue positions equivalent to amino acid residues 61 and 100 set forth in SEQ ID NO: 5. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a neurotrophic agent comprises PNT-1. In some embodiments, the neurotrophic agent comprises modifications at amino acid residue positions equivalent to amino acid residues 61 and 100 set forth in SEQ ID NO: 5. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation. In some instances, the modification at amino acid residue 61 comprises a mutation from proline to serine. In some cases, the modification at amino acid residue 100 comprises a mutation from arginine to glutamic acid.

In some embodiments, a neurotrophic agent comprises a modification at amino acid residue position equivalent to amino acid residue 118 set forth in SEQ ID NO: 5. In some embodiments, the neurotrophic agent is selected from NT-3, NT-4, NT-5, BDNF, PNT, or a chimeric neurotrophin. In some instances, the chimeric neurotrophin comprises a sequence set forth in SEQ ID NO: 6 or 7. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a neurotrophic agent is PNT. In some instances, PNT comprises a modification at amino acid residue position equivalent to amino acid residue 118 set forth in SEQ ID NO: 5. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a neurotrophic agent comprises PNT-1. In some embodiments, a neurotrophic agent comprises a modification at amino acid residue 118 set forth in SEQ ID NO: 5. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a neurotrophic agent comprises modifications at amino acid residue positions equivalent to amino acid residues 32, 34, and 35 set forth in SEQ ID NO: 6 or 7. In some embodiments, the neurotrophic agent comprises modifications at amino acid residue positions equivalent to amino acid residues 32, 34, and 35 set forth in SEQ ID NO: 6. In some embodiments, the neurotrophic agent comprises modifications at amino acid residue positions equivalent to amino acid residues 32, 34, and 35 set forth in SEQ ID NO: 7. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a non-natural neurotrophic agent comprises a modification at an amino acid residue position equivalent to at least one of an amino acid residue 3, 4, 6, 7, 8, 10, 11, 15, 17, 19, 22, 23, 24, 25, 26, 28, 31, 33, 36, 38, 40, 42, 43, 45, 46, 47, 48, 49, 51, 54, 56, 58, 59, 61, 63, 64, 65, 68, 71, 72, 73, 74, 76, 78, 80, 83, 87, 89, 91, 92, 93, 94, 95, 96, 97, 103, 105, 114, and 115 set forth in SEQ ID NO: 2. In some embodiments, the modification is at a single amino acid position set forth in SEQ ID NO: 2. In some embodiments, the modification is at multiple amino acid positions set forth in SEQ ID NO: 2. For example, the modification is at amino acid residue positions equivalent to amino acid residues 15 and 103 set forth in SEQ ID NO: 2. In some embodiments, the modification is at amino acid residue positions equivalent to amino acid residue 15, 114, and 115 set forth in SEQ ID NO: 2. In some embodiments, the modification is at amino acid residue positions equivalent to amino acid residue 11, 15, 68, 87, 114, and 115 set forth in SEQ ID NO: 2. In some embodiments, the modification is at amino acid residue positions equivalent to amino acid residue 15, 68, 87, 114, and 115 set forth in SEQ ID NO: 2. In some embodiments, the modification is at amino acid residue positions equivalent to amino acid residue 11, 15, 87, 114, and 115 set forth in SEQ ID NO: 2. In some embodiments, the modification is at amino acid residue positions equivalent to amino acid residue 15, 87, 114, and 115 set forth in SEQ ID NO: 2. In some embodiments, the modification is at amino acid residue positions equivalent to amino acid residue 11, 15, 114, and 115 set forth in SEQ ID NO: 2. In some embodiments, the modification is at amino acid residue positions equivalent to amino acid residue 15, 103, and 51 set forth in SEQ ID NO: 2. In some embodiments, the modification is at amino acid residue positions equivalent to amino acid residue 15, 103, 114, and 115 set forth in SEQ ID NO: 2. In some embodiments, the modification is at amino acid residue positions equivalent to amino acid residue 31 set forth in SEQ ID NO: 2. In some embodiments, the modification is at amino acid residue positions equivalent to amino acid residue 40 set forth in SEQ ID NO: 2. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a neurotrophic agent comprises a modification at an amino acid residue position equivalent to at least one of an amino acid residue 11, 15, 31, 40, 51, 68, 87, 103, 114, and 115 set forth in SEQ ID NO: 2. In some embodiments, the modification is at a single amino acid position set forth in SEQ ID NO: 2. In some embodiments, the modification is at multiple amino acid positions set forth in SEQ ID NO: 2. For example, the modification is at amino acid residue positions equivalent to amino acid residues 15 and 103 set forth in SEQ ID NO: 2. In some embodiments, the modification is at amino acid residue positions equivalent to amino acid residue 15, 114, and 115 set forth in SEQ ID NO: 2. In some embodiments, the modification is at amino acid residue positions equivalent to amino acid residue 11, 15, 68, 87, 114, and 115 set forth in SEQ ID NO: 2. In some embodiments, the modification is at amino acid residue positions equivalent to amino acid residue 15, 68, 87, 114, and 115 set forth in SEQ ID NO: 2. In some embodiments, the modification is at amino acid residue positions equivalent to amino acid residue 11, 15, 87, 114, and 115 set forth in SEQ ID NO: 2. In some embodiments, the modification is at amino acid residue positions equivalent to amino acid residue 15, 87, 114, and 115 set forth in SEQ ID NO: 2. In some embodiments, the modification is at amino acid residue positions equivalent to amino acid residue 11, 15, 114, and 115 set forth in SEQ ID NO: 2. In some embodiments, the modification is at amino acid residue positions equivalent to amino acid residue 15, 103, and 51 set forth in SEQ ID NO: 2. In some embodiments, the modification is at amino acid residue positions equivalent to amino acid residue 15, 103, 114, and 115 set forth in SEQ ID NO: 2. In some embodiments, the modification is at amino acid residue positions equivalent to amino acid residue 31 set forth in SEQ ID NO: 2. In some embodiments, the modification is at amino acid residue positions equivalent to amino acid residue 41 set forth in SEQ ID NO: 2. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a neurotrophic agent is NT-3. In some instances, NT-3 comprises a modification at an amino acid residue position equivalent to at least one of an amino acid residue 11, 15, 31, 40, 51, 68, 87, 103, 114, and 115 set forth in SEQ ID NO: 2. In some embodiments, the modification is at a single amino acid position set forth in SEQ ID NO: 2. In some embodiments, the modification is at multiple amino acid positions set forth in SEQ ID NO: 2. For example, the modification is at amino acid residue positions equivalent to amino acid residues 15 and 103 set forth in SEQ ID NO: 2. In some embodiments, the modification is at amino acid residue positions equivalent to amino acid residue 15, 114, and 115 set forth in SEQ ID NO: 2. In some embodiments, the modification is at amino acid residue positions equivalent to amino acid residue 11, 15, 68, 87, 114, and 115 set forth in SEQ ID NO: 2. In some embodiments, the modification is at amino acid residue positions equivalent to amino acid residue 15, 68, 87, 114, and 115 set forth in SEQ ID NO: 2. In some embodiments, the modification is at amino acid residue positions equivalent to amino acid residue 11, 15, 87, 114, and 115 set forth in SEQ ID NO: 2. In some embodiments, the modification is at amino acid residue positions equivalent to amino acid residue 15, 87, 114, and 115 set forth in SEQ ID NO: 2. In some embodiments, the modification is at amino acid residue positions equivalent to amino acid residue 11, 15, 114, and 115 set forth in SEQ ID NO: 2. In some embodiments, the modification is at amino acid residue positions equivalent to amino acid residue 15, 103, and 51 set forth in SEQ ID NO: 2. In some embodiments, the modification is at amino acid residue positions equivalent to amino acid residue 15, 103, 114, and 115 set forth in SEQ ID NO: 2. In some embodiments, the modification is at amino acid residue positions equivalent to amino acid residue 31 set forth in SEQ ID NO: 2. In some embodiments, the modification is at amino acid residue positions equivalent to amino acid residue 40 set forth in SEQ ID NO: 2. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a neurotrophic agent is the mature form of NT-3. In some embodiments, the mature form of NT-3 comprises a modification at an amino acid residue 11, 15, 31, 40, 51, 68, 87, 103, 114, and 115 set forth in SEQ ID NO: 2. In some embodiments, the modification is at a single amino acid position set forth in SEQ ID NO: 2. In some embodiments, the modification is at multiple amino acid positions set forth in SEQ ID NO: 2. For example, the modification is at amino acid residues 15 and 103 set forth in SEQ ID NO: 2. In some embodiments, the modification is at amino acid residues 15, 114, and 115 set forth in SEQ ID NO: 2. In some embodiments, the modification is at amino acid residues 11, 15, 68, 87, 114, and 115 set forth in SEQ ID NO: 2. In some embodiments, the modification is at amino acid residues 15, 68, 87, 114, and 115 set forth in SEQ ID NO: 2. In some embodiments, the modification is at amino acid residues 11, 15, 87, 114, and 115 set forth in SEQ ID NO: 2. In some embodiments, the modification is at amino acid residues 15, 87, 114, and 115 set forth in SEQ ID NO: 2. In some embodiments, the modification is at amino acid residues 11, 15, 114, and 115 set forth in SEQ ID NO: 2. In some embodiments, the modification is at amino acid residues 15, 103, and 51 set forth in SEQ ID NO: 2. In some embodiments, the modification is at amino acid residues 15, 103, 114, and 115 set forth in SEQ ID NO: 2. In some embodiments, the modification is at amino acid residues 31 set forth in SEQ ID NO: 2. In some embodiments, the modification is at amino acid residues 40 set forth in SEQ ID NO: 2. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a neurotrophic agent described herein comprises about 50%, 55%, 60%, 65%, 70%, 75%, 80%, 85%, 90%, 95%, 96%, 97%, 98%, 99%, or 100% sequence identity to the amino acid sequence set forth in SEQ ID NO: 8, 9, 10, or 11. In some embodiments, the neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 8, 9, 10, or 11. In some cases, the neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 8. In some cases, the neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 9. In some cases, the neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 10. In some cases, the neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 11. In some embodiments, a neurotrophic agent described herein comprises an amino acid sequence set forth in SEQ ID NO: 8, 9, 10, or 11.

In some embodiments, the neurotrophic agent comprises about 50%, 55%, 60%, 65%, 70%, 75%, 80%, 85%, 90%, 95%, 96%, 97%, 98%, 99%, or 100% sequence identity to the amino acid sequence set forth in SEQ ID NO: 13, 14, 15, or 16. In some embodiments, the neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 13, 14, 15, or 16. In some embodiments, the neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 13. In some embodiments, the neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 14. In some embodiments, the neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 15. In some embodiments, the neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 16. In some embodiments, a neurotrophic agent described herein comprises the amino acid sequence set forth in SEQ ID NO: 13, 14, 15, or 16.

In some embodiments, the neurotrophic agent comprises about 50%, 55%, 60%, 65%, 70%, 75%, 80%, 85%, 90%, 95%, 96%, 97%, 98%, 99%, or 100% sequence identity to the amino acid sequence set forth in SEQ ID NO: 17 or 18. In some embodiments, the neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 17 or 18. In some embodiments, the neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 17. In some embodiments, the neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 18. In some embodiments, a neurotrophic agent described herein comprises the amino acid sequence set forth in SEQ ID NO: 17 or 18.

In some embodiments, the neurotrophic agent comprises about 50%, 55%, 60%, 65%, 70%, 75%, 80%, 85%, 90%, 95%, 96%, 97%, 98%, 99%, or 100% sequence identity to the amino acid sequence set forth in SEQ ID NO: 19, 20, or 21. In some embodiments, the neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 19, 20, or 21. In some embodiments, the neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 19. In some embodiments, the neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 20. In some embodiments, the neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 21. In some embodiments, a neurotrophic agent described herein comprises the amino acid sequence set forth in SEQ ID NO: 19, 20, or 21.

In some embodiments, the neurotrophic agent comprises about 50%, 55%, 60%, 65%, 70%, 75%, 80%, 85%, 90%, 95%, 96%, 97%, 98%, 99%, or 100% sequence identity to the amino acid sequence set forth in SEQ ID NO: 22, 23, 24, 25, 26, 27, 28, 29, 30, 31, 32, 33, or 34. In some embodiments, the neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 22, 23, 24, 25, 26, 27, 28, 29, 30, 31, 32, 33, or 34. In some embodiments, the neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 22. In some embodiments, the neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 23. In some embodiments, the neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 24. In some embodiments, the neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 25. In some embodiments, the neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 26. In some embodiments, the neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 27. In some embodiments, the neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 28. In some embodiments, the neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 29. In some embodiments, the neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 30. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 31. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 32. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 33. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 34. In some embodiments, a non-natural neurotrophic agent described herein comprises the amino acid sequence set forth in SEQ ID NO: 22, 23, 24, 25, 26, 27, 28, 29, 30, 31, 32, 33, or 34.

In some embodiments, the neurotrophic agent comprises a sequence as illustrated in Table 2. In some embodiments, the neurotrophic agent consists of a sequence as illustrated in Table 2.

TABLE 2

| | | | SEQ ID |
|---|---|---|---|
| Mutein name | mutations | Sequence | NO: |
| 7-84-103 | F7A/H84A/R103A | SSSHPIAHRGEFSVCDSVSVWVGDKTTATDI | 8 |
| | | KGKEVMVLGEVNINNSVFKQYFFETKCRDP | |
| | | NPVDSGCRGIDSKHWNSYCTTTATFVKALT | |
| | | MDGKQAAWRFIAIDTACVCVLSRKAVRRA | |
| 7-45-103 | F7A/N45A/R103A | SSSHPIAHRGEFSVCDSVSVWVGDKTTATDI | 9 |
| | | KGKEVMVLGEVNIANSVFKQYFFETKCRDP | |
| | | NPVDSGCRGIDSKHWNSYCTTTHTFVKALT | |
| | | MDGKQAAWRFIAIDTACVCVLSRKAVRRA | |
| K84 | K32A/K34A/E35A | SSSHPIAHRGEFSVCDSVSVWVGDKTTATDI | 10 |
| | F7A/H84A/R103A | AGAAVMVLGEVNINNSVFKQYFFETKCRDP | |
| | | NPVDSGCRGIDSKHWNSYCTTTATFVKALT | |
| | | MDGKQAAWRFIAIDTACVCVLSRKAVRRA | |
| K45 | K32A/K34A/E35A | SSSHPIAHRGEFSVCDSVSVWVGDKTTATDI | 11 |
| | F7A/N45A/R103A | AGAAVMVLGEVNIANSVFKQYFFETKCRDP | |
| | | NPVDSGCRGIDSKHWNSYCTTTHTFVKALT | |
| | | MDGKQAAWRFIAIDTACVCVLSRKAVRRA | |
| K9 | K32A/K34A/E35A | SSSHPIFHVCDSVSVWVGDKTTATDIAGAAV | 12 |
| | Δ9-13 | MVLGEVNINNSVFKQYFFETKCRDPNPVDSG | |
| | | CRGIDSKHWNSYCTTTHTFVKALTMDGKQA | |
| | | AWRFIRIDTACVCVLSRKAVRRA | |
| K1-PNT-1 | R32A/H34A | SSSHPIFHRGEYSVCDSESLWVTDKSSAIDIA | 13 |
| | | GAQVTVLGEIKTGNSPVKQYFYETRCKEARP | |
| | | VKNGCRGIDDKHWNSQCKTSQTYVRALTSE | |
| | | SKKRIGWRWIRIDTSCVCALSRKIGRT | |
| K2-PNT-1 | K74A/H75A/N77A | SSSHPIFHRGEYSVCDSESLWVTDKSSAIDIR | 14 |
| | | GHQVTVLGEIKTGNSPVKQYFYETRCKEARP | |
| | | VKNGCRGIDDAAWASQCKTSQTYVRALTSE | |
| | | SKKRIGWRWIRIDTSCVCALSRKIGRT | |
| K3-PNT-1 | R115A/K116A | SSSHPIFHRGEYSVCDSESLWVTDKSSAIDIR | 15 |
| | | GHQVTVLGEIKTGNSPVKQYFYETRCKEARP | |
| | | VKNGCRGIDDKHWNSQCKTSQTYVRALTSE | |
| | | SKKRIGWRWIRIDTSCVCALSAAIGRT | |
| K4-PNT-1 | R32A/H34A/K74A/ | SSSHPIFHRGEYSVCDSESLWVTDKSSAIDIA | 16 |
| | H75A/ | GAQVTVLGEIKTGNSPVKQYFYETRCKEARP | |
| | N77A/R115A/K116 | VKNGCRGIDDAAWASQCKTSQTYVRALTSE | |
| | A | SKKRIGWRWIRIDTSCVCALSAAIGRT | |
| NGF/BDNF | K32A/K34A/E35A | SSSHPIFHRGEFSVCDSVSVWVGDKTTATDIA | 17 |
| chimera 3 | | GAAVMVLGEVNINNSVFKQYFFETKCNPMG | |
| (3 + 4 + 5 and | | YTKEGCRGIDSKHWNSQCRTTQSYVRALTM | |
| KKE) | | DSKKRIGWRFIRIDTACVCVLSRKAVRRA | |
| NGF/BDNF | K32A/K34A/E35A | SSSHPIFHRGEFSVCDSVSVWVTAADKKTAV | 18 |
| chimera 4 | | DMAGAAVMVLGEVNINNSVFKQYFFETKCN | |
| (1 + 3 + 4 + 5 and | | PMGYTKEGCRGIDSKHWNSQCRTTQSYVRA | |
| KKE) | | LTMDSKKRIGWRFIRIDTACVCVLSRKAVRR | |
| | | A | |
| PNT1 + KKE | R32A/H34A/Q35A | SSSHPIFHRGEYSVCDSESLWVTDKSSAIDIA | 19 |
| | | GAAVTVLGEIKTGNSPVKQYFYETRCKEARP | |
| | | VKNGCRGIDDKHWNSQCKTSQTYVRALTSE | |
| | | SKKRIGWRWIRIDTSCVCALSRKIGRT | |
| PNT-1 mutant | A61S/R101E | SSSHPIFHRGEYSVCDSESLWVTDKSSAIDIR | 20 |
| 5 | | GHQVTVLGEIKTGNSPVKQYFYETRCKESRP | |
| | | VKNGCRGIDDKHWNSQCKTSQTYVRALTSE | |
| | | SKKRIGWEWIRIDTSCVCALSRKIGRT | |
| PNT-1 mutant | G118A | SSSHPIFHRGEYSVCDSESLWVTDKSSAIDIR | 21 |
| 6 | | GHQVTVLGEIKTGNSPVKQYFYETRCKEARP | |
| | | VKNGCRGIDDKHWNSQCKTSQTYVRALTSE | |
| | | SKKRIGWRWIRIDTSCVCALSRKIART | |
| NT-3 mutant 1 | D15A/R103A | YAEHKSHRGEYSVCASESLWVTDKSSAIDIR | 22 |
| | | GHQVTVLGEIKTGNSPVKQYFYETRCKEARP | |
| | | VKNGCRGIDDKHWNSQCKTSQTYVRALTSE | |
| | | NNKLVGWRWIAIDTSCVCALSRKIGRT | |
| NT-3 mutant 2 | D15A/R114A/K115 | YAEHKSHRGEYSVCASESLWVTDKSSAIDIR | 23 |
| | A | GHQVTVLGEIKTGNSPVKQYFYETRCKEARP | |
| | | VKNGCRGIDDKHWNSQCKTSQTYVRALTSE | |
| | | NNKLVGWRWIRIDTSCVCALSAAIGRT | |
| NT-3 mutant 3 | Y11A/D15A/R68A/R | YAEHKSHRGEASVCASESLWVTDKSSAIDIR | 24 |
| | 87A/R114A/K115A | GHQVTVLGEIKTGNSPVKQYFYETRCKEARP | |
| | | VKNGCAGIDDKHWNSQCKTSQTYVAALTSE | |
| | | NNKLVGWRWIRIDTSCVCALSAAIGRT | |
| NT-3 mutant 4 | D15A/R68A/R87A/R | YAEHKSHRGEYSVCASESLWVTDKSSAIDIR | 25 |
| | 114A/K115A | GHQVTVLGEIKTGNSPVKQYFYETRCKEARP | |
| | | VKNGCAGIDDKHWNSQCKTSQTYVAALTSE | |
| | | NNKLVGWRWIRIDTSCVCALSAAIGRT | |
| NT-3 | Y11A/D15A/R87A/R | YAEHKSHRGEASVCASESLWVTDKSSAIDIR | 26 |
| mutant 5 | 114A/K115A | GHQVTVLGEIKTGNSPVKQYFYETRCKEARP | |
| | | VKNGCRGIDDKHWNSQCKTSQTYVAALTSE | |
| | | NNKLVGWRWIRIDTSCVCALSAAIGRT | |
| NT-3 mutant 6 | D15A/R87A/R114A/ | YAEHKSHRGEYSVCASESLWVTDKSSAIDIR | 27 |
| | K115A | GHQVTVLGEIKTGNSPVKQYFYETRCKEARP | |
| | | VKNGCRGIDDKHWNSQCKTSQTYVAALTSE | |
| | | NNKLVGWRWIRIDTSCVCALSAAIGRT | |
| NT-3 mutant 7 | Y11A/D15A/R114A/ | YAEHKSHRGEASVCASESLWVTDKSSAIDIR | 28 |
| | K115A | GHQVTVLGEIKTGNSPVKQYFYETRCKEARP | |
| | | VKNGCRGIDDKHWNSQCKTSQTYVRALTSE | |
| | | NNKLVGWRWIRIDTSCVCALSAAIGRT | |
| NT-3 mutant 8 | D15A/R103A/Y51A | YAEHKSHRGEYSVCASESLWVTDKSSAIDIR | 29 |
| | | GHQVTVLGEIKTGNSPVKQAFYETRCKEARP | |
| | | VKNGCRGIDDKHWNSQCKTSQTYVRALTSE | |
| | | NNKLVGWRWIAIDTSCVCALSRKIGRT | |
| NT-3 mutant 9 | D15A/R103A/R114A | YAEHKSHRGEYSVCASESLWVTDKSSAIDIR | 30 |
| | /K115A | GHQVTVLGEIKTGNSPVKQYFYETRCKEARP | |
| | | VKNGCRGIDDKHWNSQCKTSQTYVRALTSE | |
| | | NNKLVGWRWIAIDTSCVCALSAAIGRT | |
| NT-3 mutant | D15A | YAEHKSHRGEYSVCASESLWVTDKSSAIDIR | 31 |
| 10 | | GHQVTVLGEIKTGNSPVKQYFYETRCKEARP | |
| | | VKNGCRGIDDKHWNSQCKTSQTYVRALTSE | |
| | | NNKLVGWRWIRIDTSCVCALSRKIGRT | |
| NT-3 mutant | R114A/K115A | YAEHKSHRGEYSVCDSESLWVTDKSSAIDIR | 32 |
| 11 | | GHQVTVLGEIKTGNSPVKQYFYETRCKEARP | |
| | | VKNGCRGIDDKHWNSQCKTSQTYVRALTSE | |
| | | NNKLVGWRWIRIDTSCVCALSAAIGRT | |
| NT-3 mutant | R31A | YAEHKSHRGEYSVCASESLWVTDKSSAIDIA | 33 |
| 12 | | GHQVTVLGEIKTGNSPVKQYFYETRCKEARP | |
| | | VKNGCRGIDDKHWNSQCKTSQTYVRALTSE | |
| | | NNKLVGWRWIRIDTSCVCALSRKIGRT | |
| NT-3 mutant | E40A | YAEHKSHRGEYSVCASESLWVTDKSSAIDIA | 34 |
| 13 | | GHQVTVLGAIKTGNSPVKQYFYETRCKEARP | |
| | | VKNGCRGIDDKHWNSQCKTSQTYVRALTSE | |
| | | NNKLVGWRWIRIDTSCVCALSRKIGRT | |

In some embodiments, the neurotrophic agent is GDNF. In certain instances, GDNF and BDNF are neurotrophic agents that promote the survival of existing neurons and their processes and connections by repairing damaged cells, inhibiting the production of ROS, and/or inhibiting cell death. In some embodiments, the neurotrophic agent also promotes the differentiation of neural cell progenitors. Further, in some embodiments, the neurotrophic agent protects the Cranial Nerve VIII from degeneration. In some embodiments, cells treated with exogenous GDNF have higher survival rates after trauma than untreated cells. In some embodiments, the neurotrophic agent BDNF is administered in conjunction with fibroblast growth factor. In some cases, GDNF comprises a naturally occurring GDNF with one or more mutations or modifications (e.g., chemical modifications such as phosphorylation, incorporation of unnatural amino acids, biotinylation, cyclisation, and the like) in amino acid residues.

In some embodiments, the neurotrophic agent is CNTF. In some embodiments, CNTF promotes the synthesis of neurotransmitters and the growth of neurites. In some embodiments, CNTF is administered in conjunction with BDNF. In some cases, CNTF comprises a naturally occurring CNTF with one or more mutations or modifications (e.g., chemical modifications such as phosphorylation, incorporation of unnatural amino acids, biotinylation, cyclisation, and the like) in amino acid residues.

In some embodiments, binding of a Trk agonist to a Trk receptor results in increased levels of phosphorylated Trk receptor, phosphorylated MAPK, phosphorylated Akt, phosphorylated ERK1/2, and phosphorylated phospholipase C-γ. In some embodiments, binding of a Trk agonist to Trk receptor leads to improved neuronal survival. In some embodiments, administration of a non-otic composition comprising a Trk agonist that binds to Trk receptor leads to improved neuronal survival and treats or prevents a non-otic condition. In some embodiments, administration of a non-otic composition comprising a Trk agonist that binds to a Trk receptor leads to improved neuronal survival and treats or prevents a non-otic condition that requires reconnection of afferent sensory fibers and repair of synapses. In some embodiments, administration of a non-otic composition comprising a Trk agonist that binds to a Trk receptor treats or prevents a non-otic condition such as a neurodegenerative disease or disorder. In some embodiments, administration of a non-otic composition comprising a Trk agonist that binds to a Trk receptor leads to improved neuronal survival.

In some embodiments, binding of a Trk agonist to a Trk receptor has a trophic effect. In some embodiments, the trophic effect is at least one of promotion of healthy growth of cells and wound healing. In some embodiments, binding of the Trk agonist to the Trk receptor promotes at least one of differentiation, proliferation, and growth of stem cells such as neural stem cells. In some instances, the stem cells are treated with the Trk agonist ex vivo.

In some embodiments, the binding affinity of a Trk agonist to a Trk receptor is from about 0.001 nM to about 60 μM. In some instances, the binding affinity of a Trk agonist to a Trk receptor is from about 0.001 nM to about 60 μM, 0.001 nM to about 50 μM, 0.001 nM to about 40 μM, 0.001 nM to about 30 μM, 0.001 nM to about 20 μM, 0.001 nM to about 10 μM, 0.001 nM to about 8 μM, 0.001 nM to about 5 μM 0.01 nM to about 50 μM, 0.001 nM to about 3 μM, 0.001 nM to about 1 μM, 0.01 nM to about 50 μM from about 0.01 nM to about 40 μM, from about 0.01 nM to about 30 μM, from about 0.01 nM to about 20 μM, from about 0.01 nM to about 10 μM, from about 0.01 nM to about 8 μM, from about 0.01 nM to about 5 μM, from about 0.01 nM to about 3 μM, from about 0.01 nM to about 1 μM, from about 0.1 nM to about 50 μM, from about 0.1 nM to about 40 μM, from about 0.1 nM to about 30 μM, from about 0.1 nM to about 20 μM, from about 0.1 nM to about 10 μM, from about 0.1 nM to about 8 μM, from about 0.1 nM to about 5 μM, from about 0.01 nM to about 1 μM, from about 1 nM to about 50 μM, from about 10 nM to about 30 μM, from about 100 nM to about 20 μM, from about 500 nM to about 10 μM, from about 500 nM to about 1 μM, from about 50 nM to about 5 μM, from about 100 nM to about 1 μM, or from about 1 nM to about 1 μM. In some embodiments, the Trk receptor is at least one of TrkA, TrkB, and TrkC.

In some embodiments, the binding affinity of a Trk agonist to a Trk receptor is from about 0.10 to about 0.80 nM, from about 0.15 to about 0.75 nM, from about 0.18 to about 0.72 nM, from about 1 nM to about 1.5 nM, from about 2 nM to about 5 nM, from about 10 nM to about 20 nM, from about 30 nM to about 50 nM, from about 75 nM to about 100 nM, from about 125 nM to about 150 nM, or from about 160 nM to about 200 nM. In some embodiments, the binding affinity is about 2 pM, about 5 pM, about 10 pM, about 15 pM, about 20 pM, about 40 pM, or greater than about 40 pM. In some embodiments, the binding affinity is between about 2 pM and 22 pM. In some embodiments, the binding affinity is less than about 10 nM, about 5 nM, about 1 nM, about 900 pM, about 800 pM, about 700 pM, about 600 pM, about 500 pM, about 400 pM, about 300 pM, about 200 pM, about 150 pM, about 100 pM, about 90 pM, about 80 pM, about 70 pM, about 60 pM, about 50 pM, about 40 pM, about 30 pM, about 10 pM. In some embodiments, the binding affinity is about 10 nM. In some embodiments, the binding affinity is less than about 10 nM. In other embodiments, the binding affinity is about 0.1 nM or about 0.07 nM. In other embodiments, the binding affinity is less than about 0.1 nM or less than about 0.07 nM. In some embodiments, the binding affinity is any of about 10 nM, about 5 nM, about 1 nM, about 900 pM, about 800 pM, about 700 pM, about 600 pM, about 500 pM, about 400 pM, about 300 pM, about 200 pM, about 150 pM, about 100 pM, about 90 pM, about 80 pM, about 70 pM, about 60 pM, about 50 pM, about 40 pM, about 30 pM, about 10 pM, about 5 pM, or about 2 pM. In some embodiments, the binding affinity is any of about 10 nM, about 5 nM, about 1 nM, about 900 pM, about 800 pM, about 700 pM, about 600 pM, about 500 pM, about 400 pM, about 300 pM, about 200 pM, about 150 pM, about 100 pM, about 90 pM, about 80 pM, about 70 pM, about 60 pM, about 50 pM, about 40 pM, about 30 pM, or about 10 pM. In some embodiments, the binding affinity is about 2 pM, about 5 pM, about 10 pM, about 15 pM, about 20 pM, about 40 pM, or greater than about 40 pM. In some embodiments, the binding affinity falls within any range bound by any of these values, for example, from about 175 nM to about 180 nM. In some embodiments, the binding affinity is 100 nM. In some embodiments, the binding affinity is 200 nM. In some embodiments, the Trk receptor is at least one of TrkA, TrkB, and TrkC.

In some embodiments, the potency of a Trk agonist to a Trk receptor is from about 0.001 nM to about 60 μM. In some instances, the potency of a Trk agonist to a Trk receptor is from about 0.001 nM to about 60 μM, 0.001 nM to about 50 μM, 0.001 nM to about 40 μM, 0.001 nM to about 30 μM, 0.001 nM to about 20 μM, 0.001 nM to about 10 μM, 0.001 nM to about 8 μM, 0.001 nM to about 5 μM 0.01 nM to about 50 μM, 0.001 nM to about 3 μM, 0.001 nM to about 1 μM, 0.01 nM to about 50 μM, from about 0.01 nM to about 40 μM, from about 0.01 nM to about 30 μM, from about 0.01 nM to about 20 μM, from about 0.01 nM to about 10 μM, from about 0.01 nM to about 8 μM, from about 0.01 nM to about 5 μM, from about 0.01 nM to about 3 μM, from about 0.01 nM to about 1 μM, from about 0.1 nM to about 50 μM, from about 0.1 nM to about 40 μM, from about 0.1 nM to about 30 μM, from about 0.1 nM to about 20 μM, from about 0.1 nM to about 10 μM, from about 0.1 nM to about 8 μM, from about 0.1 nM to about 5 μM, from about 0.01 nM to about 1 μM, from about 1 nM to about 50 μM, from about 10 nM to about 30 μM, from about 100 nM to about 20 μM, from about 500 nM to about 10 μM, from about 500 nM to about 1 μM, from about 50 nM to about 5 μM, from about 100 nM to about 1 μM, or from about 1 nM to about 1 μM. In some embodiments, the Trk receptor is at least one of TrkA, TrkB, and TrkC.

In some embodiments, the potency of a Trk agonist to a Trk receptor is from about 0.10 to about 0.80 nM, from about 0.15 to about 0.75 nM, from about 0.18 to about 0.72 nM, from about 1 nM to about 1.5 nM, from about 2 nM to about 5 nM, from about 10 nM to about 20 nM, from about 30 nM to about 50 nM, from about 75 nM to about 100 nM, from about 125 nM to about 150 nM, or from about 160 nM to about 200 nM. In some embodiments, the potency is about 2 pM, about 5 pM, about 10 pM, about 15 pM, about 20 pM, about 40 pM, or greater than about 40 pM. In some embodiments, the potency is between about 2 pM and 22 pM. In some embodiments, the potency is less than about 10 nM, about 5 nM, about 1 nM, about 900 pM, about 800 pM, about 700 pM, about 600 pM, about 500 pM, about 400 pM, about 300 pM, about 200 pM, about 150 pM, about 100 pM, about 90 pM, about 80 pM, about 70 pM, about 60 pM, about 50 pM, about 40 pM, about 30 pM, about 10 pM. In some embodiments, the potency is about 10 nM. In some embodiments, the potency is less than about 10 nM. In other embodiments, the potency is about 0.1 nM or about 0.07 nM. In other embodiments, the potency is less than about 0.1 nM or less than about 0.07 nM. In some embodiments, the potency is any of about 10 nM, about 5 nM, about 1 nM, about 900 pM, about 800 pM, about 700 pM, about 600 pM, about 500 pM, about 400 pM, about 300 pM, about 200 pM, about 150 pM, about 100 pM, about 90 pM, about 80 pM, about 70 pM, about 60 pM, about 50 pM, about 40 pM, about 30 pM, about 10 pM, about 5 pM, or about 2 pM. In some embodiments, the potency is any of about 10 nM, about 5 nM, about 1 nM, about 900 pM, about 800 pM, about 700 pM, about 600 pM, about 500 pM, about 400 pM, about 300 pM, about 200 pM, about 150 pM, about 100 pM, about 90 pM, about 80 pM, about 70 pM, about 60 pM, about 50 pM, about 40 pM, about 30 pM, or about 10 pM. In some embodiments, the potency is about 2 pM, about 5 pM, about 10 pM, about 15 pM, about 20 pM, about 40 pM, or greater than about 40 pM. In some embodiments, the potency falls within any range bound by any of these values, for example, from about 175 nM to about 180 nM. In some embodiments, the potency is 100 nM. In some embodiments, the potency is 200 nM. In some embodiments, the Trk receptor is at least one of TrkA, TrkB, and TrkC.

In some embodiments, the off-rate (or k_{off}) of a Trk agonist to Trk receptor is from about 10⁻¹ to about 10⁻⁶ s⁻¹. In some embodiments, the off-rate (or k_{off}) of a Trk agonist to Trk receptor is from about 10⁻² to about 10⁻⁶ s⁻¹, from about 10⁻³ to about 10⁻⁶ s⁻¹, from about 10⁴ to about 10⁻⁶ s⁻¹, from about 10⁻² to about 10⁻⁵ s⁻¹, from about 10⁻² to about 10⁻⁴ s⁻¹, from about 10⁻² to about 10⁻³ s⁻¹, from about 10⁻³ to about 10⁻⁵ s⁻¹, from about 10⁻³ to about 10⁻⁴ s⁻¹, from about 10⁴ to about 10⁻⁵ s⁻¹, from about 10⁻¹ to about 10⁻⁵ s⁻¹, from about 10⁻¹ to about 10⁻⁴ s⁻¹, from about 10⁻¹ to about 10⁻³ s⁻¹, or from about 10⁻¹ to about 10⁻² s⁻¹. In some embodiments, the off-rate (or k_{off}) of a TrkA agonist to TrkA receptor is about 10⁻¹ s⁻¹, about 10⁻² s⁻¹, about 10⁻³ s⁻¹, about 10⁻⁴ s⁻¹, about 10⁻⁵ s⁻¹, or about 10⁻⁶ s⁻¹. In some embodiments, the Trk receptor is at least one of TrkA, TrkB, and TrkC.

In some embodiments, a neurotrophic agent described herein has an increased binding affinity to one or more Trk receptors relative to the binding affinity of an equivalent wild type neurotrophin. In some instances, the increase in binding affinity is about 20%, 30%, 40%, 50%, 60%, 70%, 80%, 85%, 90%, 95%, 96%, 97%, 98%, 99%, or more, relative to the binding affinity of an equivalent wild type neurotrophin. In some cases, the increase in binding affinity is about 20% or more, relative to the binding affinity of an equivalent wild type neurotrophin. In some cases, the increase in binding affinity is about 30% or more, relative to the binding affinity of an equivalent wild type neurotrophin. In some cases, the increase in binding affinity is about 40% or more, relative to the binding affinity of an equivalent wild type neurotrophin. In some cases, the increase in binding affinity is about 50% or more, relative to the binding affinity of an equivalent wild type neurotrophin. In some cases, the increase in binding affinity is about 60% or more, relative to the binding affinity of an equivalent wild type neurotrophin. In some cases, the increase in binding affinity is about 70% or more, relative to the binding affinity of an equivalent wild type neurotrophin. In some cases, the increase in binding affinity is about 80% or more, relative to the binding affinity of an equivalent wild type neurotrophin. In some cases, the increase in binding affinity is about 85% or more, relative to the binding affinity of an equivalent wild type neurotrophin. In some cases, the increase in binding affinity is about 90% or more, relative to the binding affinity of an equivalent wild type neurotrophin. In some cases, the increase in binding affinity is about 95% or more, relative to the binding affinity of an equivalent wild type neurotrophin. In some cases, the increase in binding affinity is about 96% or more, relative to the binding affinity of an equivalent wild type neurotrophin. In some cases, the increase in binding affinity is about 97% or more, relative to the binding affinity of an equivalent wild type neurotrophin. In some cases, the increase in binding affinity is about 98% or more, relative to the binding affinity of an equivalent wild type neurotrophin. In some cases, the increase in binding affinity is about 99% or more, relative to the binding affinity of an equivalent wild type neurotrophin.

In some embodiments, a neurotrophic agent described herein has a decrease in binding affinity to p75^{NTR}, relative to the binding affinity of an equivalent wild-type neurotrophin. In some embodiments, a neurotrophic agent described herein or activates the p75^{NTR} with lower efficacy than naturally occurring neurotrophins. In some embodiments, the neurotrophic agent is at least one of NGF, NT-3, NT-4, NT-5, BDNF, PNT, and a chimeric neurotrophic agent. In some embodiments, the decrease in binding affinity is about 30%, 40%, 50%, 60%, 70%, 80%, 85%, 90%, 95%, 96%, 97%, 98%, 99%, or more, relative to the binding affinity of an equivalent wild type neurotrophin. In some embodiments, the decrease in binding affinity is about 30% or more, relative to the binding affinity of an equivalent wild type neurotrophin. In some embodiments, the decrease in binding affinity is about 40% or more, relative to the binding affinity of an equivalent wild type neurotrophin. In some embodiments, the decrease in binding affinity is about 50% or more, relative to the binding affinity of an equivalent wild type neurotrophin. In some embodiments, the decrease in binding affinity is about 60% or more, relative to the binding affinity of an equivalent wild type neurotrophin. In some embodiments, the decrease in binding affinity is about 70% or more, relative to the binding affinity of an equivalent wild type neurotrophin. In some embodiments, the decrease in binding affinity is about 80% or more, relative to the binding affinity of an equivalent wild type neurotrophin. In some embodiments, the decrease in binding affinity is about 85% or more, relative to the binding affinity of an equivalent wild type neurotrophin. In some embodiments, the decrease in binding affinity is about 90% or more, relative to the binding affinity of an equivalent wild type neurotrophin. In some embodiments, the decrease in binding affinity is about 95% or more, relative to the binding affinity of an equivalent wild type neurotrophin. In some embodiments, the decrease in binding affinity is about 96% or more, relative to the binding affinity of an equivalent wild type neurotrophin. In some embodiments, the decrease in binding affinity is about 97% or more, relative to the binding affinity of an equivalent wild type neurotrophin. In some embodiments, the decrease in binding affinity is about 98% or more, relative to the binding affinity of an equivalent wild type neurotrophin. In some embodiments, the decrease in binding affinity is about 99% or more, relative to the binding affinity of an equivalent wild type neurotrophin. In some embodiments, the decrease in binding affinity is more than 99%, relative to the binding affinity of an equivalent wild type neurotrophin. In some embodiments, a neurotrophic agent described herein does not bind to p75^{NTR}. In some instances, the term p75^{NTR} encompasses one or more p75^{NTR} isoforms. In some embodiments, agonist activity is determined by one or more techniques well-known in the art. Suitable techniques include, but not limited to, Western blot (Wes Protein Analyzer, Protein Simple Inc.), AlphaLisa assay (Perkin Elmer Enspire plate reader), ELISA, and immunohistochemistry. In some instances, a protein modification such as protein phosphorylation is measured in cells. For example, p-ERK and p-AKT is measured. In some instances, cell survival assays are performed. In some embodiments, assays are performed to measure neurite outgrowth and branching. In some embodiments, techniques are used with at least one of cultured cells, primary cells, and tissue. In some instances, techniques are used with cells that heterologously express a Trk receptor. In some embodiments, EC₅₀ and maximal effect values such as Vₘₐₓ are determined from dose-response curves using such techniques.

In some embodiments, the binding affinity is determined by one or more techniques well-known in the art. Suitable techniques include, e.g., surface plasmon resonance (Biacore3000™ surface plasmon resonance (SPR) system, Biacore, Inc.) equipped with pre-immobilized streptavidin sensor chips, which allows determination of the rate constants for binding (kₐ) and dissociation (k_{d}) of an agonist to a TrkC receptor; isothermal titration calorimetry (ITC); Octet® (ForteBio); KinExA® (Kinetic Exclusion Assay, KinExA 3000, Sapidyne Instruments, Inc.); flow cytometry; and ELISA.

In some embodiments, a modification disclosed herein comprises a mutation to a natural or unnatural amino acid residue. For example, in some cases, the modification comprises a mutation of an amino acid residue to an alternative natural amino acid residue, for example a mutation of an arginine to an alanine. In some instances, the modification comprises a mutation of an amino acid residue to an unnatural amino acid residue, for example, a mutation of a cysteine to a seleno-L-cystine.

As used herein, an amino acid residue is a molecule containing both an amino group and a carboxyl group. Suitable amino acids for use in a neurotrophic agent described herein include, without limitation, both the D- and L-isomers of the naturally-occurring amino acids, as well as non-naturally occurring amino acids prepared by organic synthesis or other metabolic routes. In some instances, an amino acid is an α-amino acid, β-amino acid, natural amino acid, non-natural amino acid, or amino acid analog. A naturally occurring amino acid comprises any one of the twenty amino acids commonly found in peptides synthesized in nature, and known by the one letter abbreviations A, R, N, C, D, Q, E, G, H, I, L, K, M, F, P, S, T, W, Y, and V.

Exemplary unnatural amino acid residues comprise, for example, amino acid analogs such as β-amino acid analogs; racemic analogs; or analogs of amino acid residue alanine, valine, glycine, leucine, arginine, lysine, aspartic acid, glutamic acid, cysteine, methionine, tyrosine, phenylalanine, tryptophane, serine, threonine, or proline. Exemplary β-amino acid analogs include, but are not limited to, cyclic β-amino acid analogs, β-alanine, (R)-β-phenylalanine, (R)-1,2,3,4-tetrahydro-isoquinoline-3-acetic acid, (R)-3-amino-4-(1-naphthyl)-butyric acid, (R)-3-amino-4-(2,4-dichlorophenyl)butyric acid, (R)-3-amino-4-(2-chlorophenyl)-butyric acid, (R)-3-amino-4-(2-cyanophenyl)-butyric acid, (R)-3-amino-4-(2-fluorophenyl)-butyric acid, (R)-3-amino-4-(2-furyl)-butyric acid, (R)-3-amino-4-(2-methylphenyl)-butyric acid, (R)-3-amino-4-(2-naphthyl)-butyric acid, (R)-3-amino-4-(2-thienyl)-butyric acid, (R)-3-amino-4-(2-trifluoromethylphenyl)-butyric acid, (R)-3-amino-4-(3,4-dichlorophenyl)butyric acid, (R)-3-amino-4-(3,4-difluorophenyl)butyric acid, (R)-3-amino-4-(3-benzothienyl)-butyric acid, (R)-3-amino-4-(3-chlorophenyl)-butyric acid, (R)-3-amino-4-(3-cyanophenyl)-butyric acid, (R)-3-amino-4-(3-fluorophenyl)-butyric acid, (R)-3-amino-4-(3-methylphenyl)-butyric acid, (R)-3-amino-4-(3-pyridyl)-butyric acid, (R)-3-amino-4-(3-thienyl)-butyric acid, (R)-3-amino-4-(3-trifluoromethylphenyl)-butyric acid, (R)-3-amino-4-(4-bromophenyl)-butyric acid, (R)-3-amino-4-(4-chlorophenyl)-butyric acid, (R)-3-amino-4-(4-cyanophenyl)-butyric acid, (R)-3-amino-4-(4-fluorophenyl)-butyric acid, (R)-3-amino-4-(4-iodophenyl)-butyric acid, (R)-3-amino-4-(4-methylphenyl)-butyric acid, (R)-3-amino-4-(4-nitrophenyl)-butyric acid, (R)-3-amino-4-(4-pyridyl)-butyric acid, (R)-3-amino-4-(4-trifluoromethylphenyl)-butyric acid, (R)-3-amino-4-pentafluoro-phenylbutyric acid, (R)-3-amino-5-hexenoic acid, (R)-3-amino-5-hexynoic acid, (R)-3-amino-5-phenylpentanoic acid, (R)-3-amino-6-phenyl-5-hexenoic acid, (S)-1,2,3,4-tetrahydro-isoquinoline-3-acetic acid, (S)-3-amino-4-(1-naphthyl)-butyric acid, (S)-3-amino-4-(2,4-dichlorophenyl)butyric acid, (S)-3-amino-4-(2-chlorophenyl)-butyric acid, (S)-3-amino-4-(2-cyanophenyl)-butyric acid, (S)-3-amino-4-(2-fluorophenyl)-butyric acid, (S)-3-amino-4-(2-furyl)-butyric acid, (S)-3-amino-4-(2-methylphenyl)-butyric acid, (S)-3-amino-4-(2-naphthyl)-butyric acid, (S)-3-amino-4-(2-thienyl)-butyric acid, (S)-3-amino-4-(2-trifluoromethylphenyl)-butyric acid, (S)-3-amino-4-(3,4-dichlorophenyl)butyric acid, (S)-3-amino-4-(3,4-difluorophenyl)butyric acid, (S)-3-amino-4-(3-benzothienyl)-butyric acid, (S)-3-amino-4-(3-chlorophenyl)-butyric acid, (S)-3-amino-4-(3-cyanophenyl)-butyric acid, (S)-3-amino-4-(3-fluorophenyl)-butyric acid, (S)-3-amino-4-(3-methylphenyl)-butyric acid, (S)-3-amino-4-(3-pyridyl)-butyric acid, (S)-3-amino-4-(3-thienyl)-butyric acid, (S)-3-amino-4-(3-trifluoromethylphenyl)-butyric acid, (S)-3-amino-4-(4-bromophenyl)-butyric acid, (S)-3-amino-4-(4-chlorophenyl) butyric acid, (S)-3-amino-4-(4-cyanophenyl)-butyric acid, (S)-3-amino-4-(4-fluorophenyl) butyric acid, (S)-3-amino-4-(4-iodophenyl)-butyric acid, (S)-3-amino-4-(4-methylphenyl)-butyric acid, (S)-3-amino-4-(4-nitrophenyl)-butyric acid, (S)-3-amino-4-(4-pyridyl)-butyric acid, (S)-3-amino-4-(4-trifluoromethylphenyl)-butyric acid, (S)-3-amino-4-pentafluoro-phenylbutyric acid, (S)-3-amino-5-hexenoic acid, (S)-3-amino-5-hexynoic acid, (S)-3-amino-5-phenylpentanoic acid, (S)-3-amino-6-phenyl-5-hexenoic acid, 1,2,5,6-tetrahydropyridine-3-carboxylic acid, 1,2,5,6-tetrahydropyridine-4-carboxylic acid, 3-amino-3-(2-chlorophenyl)-propionic acid, 3-amino-3-(2-thienyl)-propionic acid, 3-amino-3-(3-bromophenyl)-propionic acid, 3-amino-3-(4-chlorophenyl)-propionic acid, 3-amino-3-(4-methoxyphenyl)-propionic acid, 3-amino-4,4,4-trifluoro-butyric acid, 3-aminoadipic acid, D-β-phenylalanine, β-leucine, L-β-homoalanine, L-β-homoaspartic acid γ-benzyl ester, L-β-homoglutamic acid δ-benzyl ester, L-β-homoisoleucine, L-β-homoleucine, L-β-homomethionine, L-β-homophenylalanine, L-β-homoproline, L-β-homotryptophan, L-β-homoyaline, L-Nω-benzyloxycarbonyl-β-homolysine, Nω-L-β-homoarginine, O-benzyl-L-β-homohydroxyproline, O-benzyl-L-β-homoserine, O-benzyl-L-β-homothreonine, O-benzyl-L-β-homotyrosine, γ-trityl-L-β-homoasparagine, (R)-β-phenylalanine, L-β-homoaspartic acid γ-t-butyl ester, L-β-homoglutamic acid δ-t-butyl ester, L-Nω-β-homolysine, Nδ-trityl-L-β-homoglutamine, Nω-2,2,4,6,7-pentamethyl-dihydrobenzofuran-5-sulfonyl-L-β-homoarginine, O-t-butyl-L-β-homohydroxy-proline, O-t-butyl-L-β-homoserine, O-t-butyl-L-β-homothreonine, O-t-butyl-L-β-homotyrosine, 2-aminocyclopentane carboxylic acid, and 2-aminocyclohexane carboxylic acid.

In some instances, unnatural amino acid residues comprise a racemic mixture of amino acid analogs. For example, in some instances, the D isomer of the amino acid analog is used. In some cases, the L isomer of the amino acid analog is used. In some instances, the amino acid analog comprises chiral centers that are in the R or S configuration. Sometimes, the amino group(s) of a β-amino acid analog is substituted with a protecting group, e.g., tert-butyloxycarbonyl (BOC group), 9-fluorenylmethyloxycarbonyl (FMOC), tosyl, and the like. Sometimes, the carboxylic acid functional group of a β-amino acid analog is protected, e.g., as its ester derivative. In some cases, the salt of the amino acid analog is used.

In some cases, unnatural amino acid residues comprise analogs of amino acid residue alanine, valine, glycine, leucine, arginine, lysine, aspartic acid, glutamic acid, cysteine, methionine, tyrosine, phenylalanine, tryptophane, serine, threonine, or proline. Exemplary amino acid analogs of alanine, valine, glycine, and leucine include, but are not limited to, α-methoxyglycine, α-allyl-L-alanine, α-aminoisobutyric acid, α-methyl-leucine, β-(1-naphthyl)-D-alanine, β-(1-naphthyl)-L-alanine, β-(2-naphthyl)-D-alanine, β-(2-naphthyl)-L-alanine, β-(2-pyridyl)-D-alanine, β-(2-pyridyl)-L-alanine, β-(2-thienyl)-D-alanine, β-(2-thienyl)-L-alanine, β-(3-benzothienyl)-D-alanine, β-(3-benzothienyl)-L-alanine, β-(3-pyridyl)-D-alanine, β-(3-pyridyl)-L-alanine, β-(4-pyridyl)-D-alanine, β-(4-pyridyl)-L-alanine, β-chloro-L-alanine, β-cyano-L-alanin, β-cyclohexyl-D-alanine, β-cyclohexyl-L-alanine, β-cyclopenten-1-yl-alanine, β-cyclopentyl-alanine, β-cyclopropyl-L-Ala-OH.dicyclohexylammonium salt, β-t-butyl-D-alanine, β-t-butyl-L-alanine, γ-aminobutyric acid, L-α,β-diaminopropionic acid, 2,4-dinitro-phenylglycine, 2,5-dihydro-D-phenylglycine, 2-amino-4,4,4-trifluorobutyric acid, 2-fluoro-phenylglycine, 3-amino-4,4,4-trifluoro-butyric acid, 3-fluoro-valine, 4,4,4-trifluoro-valine, 4,5-dehydro-L-leu-OH.dicyclohexylammonium salt, 4-fluoro-D-phenylglycine, 4-fluoro-L-phenylglycine, 4-hydroxy-D-phenylglycine, 5,5,5-trifluoro-leucine, 6-aminohexanoic acid, cyclopentyl-D-Gly-OH.dicyclohexylammonium salt, cyclopentyl-Gly-OH. dicyclohexylammonium salt, D-α,β-diaminopropionic acid, D-α-aminobutyric acid, D-α-t-butylglycine, D-(2-thienyl)glycine, D-(3-thienyl)glycine, D-2-aminocaproic acid, D-2-indanylglycine, D-allylglycine-dicyclohexylammonium salt, D-cyclohexylglycine, D-norvaline, D-phenylglycine, β-aminobutyric acid, β-aminoisobutyric acid, (2-bromophenyl)glycine, (2-methoxyphenyl)glycine, (2-methylphenyl)glycine, (2-thiazoyl)glycine, (2-thienyl)glycine, 2-amino-3-(dimethylamino)-propionic acid, L-α,β-diaminopropionic acid, L-α-aminobutyric acid, L-α-t-butylglycine, L-β-thienyl)glycine, L-2-amino-3-(dimethylamino)-propionic acid, L-2-aminocaproic acid dicyclohexyl-ammonium salt, L-2-indanylglycine, L-allylglycine.dicyclohexyl ammonium salt, L-cyclohexylglycine, L-phenylglycine, L-propargylglycine, L-norvaline, N-α-aminomethyl-L-alanine, D-α,γ-diaminobutyric acid, L-α,γ-diaminobutyric acid, β-cyclopropyl-L-alanine, (N-β-(2,4-dinitrophenyl))-L-α,β-diaminopropionic acid, (N-β-1-(4,4-dimethyl-2,6-dioxocyclohex-1-ylidene)ethyl)-D-α,β-diaminopropionic acid, (N-β-1-(4,4-dimethyl-2,6-dioxocyclohex-1-ylidene)ethyl)-L-α,β-diaminopropionic acid, (N-β-4-methyltrityl)-L-α,β-diaminopropionic acid, (N-β-allyloxycarbonyl)-L-α,β-diaminopropionic acid, (N-γ-1-(4,4-dimethyl-2,6-dioxocyclohex-1-ylidene)ethyl)-D-α,γ-diaminobutyric acid, (N-γ-1-(4,4-dimethyl-2,6-dioxocyclohex-1-ylidene)ethyl)-L-α,γ-diaminobutyric acid, (N-γ-4-methyltrityl)-D-α,γ-diaminobutyric acid, (N-γ-4-methyltrityl)-L-α,γ-diaminobutyric acid, (N-γ-allyloxycarbonyl)-L-α,γ-diaminobutyric acid, D-α,γ-diaminobutyric acid, 4,5-dehydro-L-leucine, cyclopentyl-D-Gly-OH, cyclopentyl-Gly-OH, D-allylglycine, D-homocyclohexylalanine, L-1-pyrenylalanine, L-2-aminocaproic acid, L-allylglycine, L-homocyclohexylalanine, and N-(2-hydroxy-4-methoxy-Bzl)-Gly-OH.

Exemplary amino acid analogs of arginine and lysine include, but are not limited to, citrulline, L-2-amino-3-guanidinopropionic acid, L-2-amino-3-ureidopropionic acid, L-citrulline, Lys(Me)₂-OH, Lys(N₃)—OH, Nδ-benzyloxycarbonyl-L-ornithine, Nω-nitro-D-arginine, Nω-nitro-L-arginine, α-methyl-ornithine, 2,6-diaminoheptanedioic acid, L-ornithine, (Nδ-1-(4,4-dimethyl-2,6-dioxo-cyclohex-1-ylidene)ethyl)-D-ornithine, (Nδ-1-(4,4-dimethyl-2,6-dioxo-cyclohex-1-ylidene)ethyl)-L-ornithine, (Nδ-4-methyltrityl)-D-ornithine, (Nδ-4-methyltrityl)-L-ornithine, D-ornithine, L-ornithine, Arg(Me)(Pbf)-OH, Arg(Me)₂-OH (asymmetrical), Arg(Me)₂-OH (symmetrical), Lys(ivDde)-OH, Lys(Me)2-OH.HCl, Lys(Me3)-OH chloride, Nω-nitro-D-arginine, and Nω-nitro-L-arginine.

Exemplary amino acid analogs of aspartic and glutamic acids include, but are not limited to, α-methyl-D-aspartic acid, α-methyl-glutamic acid, α-methyl-L-aspartic acid, γ-methylene-glutamic acid, (N-γ-ethyl)-L-glutamine, [N-α-(4-aminobenzoyl)]-L-glutamic acid, 2,6-diaminopimelic acid, L-α-aminosuberic acid, D-2-aminoadipic acid, D-α-aminosuberic acid, α-aminopimelic acid, iminodiacetic acid, L-2-aminoadipic acid, threo-β-methyl-aspartic acid, γ-carboxy-D-glutamic acid γ,γ-di-t-butyl ester, γ-carboxy-L-glutamic acid γ,γ-di-t-butyl ester, Glu(OAll)-OH, L-Asu(OtBu)-OH, and pyroglutamic acid.

Exemplary amino acid analogs of cysteine and methionine include, but are not limited to, Cys(farnesyl)-OH, Cys(farnesyl)-OMe, α-methyl-methionine, Cys(2-hydroxyethyl)-OH, Cys(3-aminopropyl)-OH, 2-amino-4-(ethylthio)butyric acid, buthionine, buthioninesulfoximine, ethionine, methionine methylsulfonium chloride, selenomethionine, cysteic acid, [2-(4-pyridyl)ethyl]-DL-penicillamine, [2-(4-pyridyl)ethyl]-L-cysteine, 4-methoxybenzyl-D-penicillamine, 4-methoxybenzyl-L-penicillamine, 4-methylbenzyl-D-penicillamine, 4-methylbenzyl-L-penicillamine, benzyl-D-cysteine, benzyl-L-cysteine, benzyl-DL-homocysteine, carbamoyl-L-cysteine, carboxyethyl-L-cysteine, carboxymethyl-L-cysteine, diphenylmethyl-L-cysteine, ethyl-L-cysteine, methyl-L-cysteine, t-butyl-D-cysteine, trityl-L-homocysteine, trityl-D-penicillamine, cystathionine, homocystine, L-homocystine, (2-aminoethyl)-L-cysteine, seleno-L-cystine, cystathionine, Cys(StBu)-OH, and acetamidomethyl-D-penicillamine.

Exemplary amino acid analogs of phenylalanine and tyrosine include, but are not limited to, β-methyl-phenylalanine, β-hydroxyphenylalanine, α-methyl-3-methoxy-DL-phenylalanine, α-methyl-D-phenylalanine, α-methyl-L-phenylalanine, 1,2,3,4-tetrahydroisoquinoline-3-carboxylic acid, 2,4-dichloro-phenylalanine, 2-(trifluoromethyl)-D-phenylalanine, 2-(trifluoromethyl)-L-phenylalanine, 2-bromo-D-phenylalanine, 2-bromo-L-phenylalanine, 2-chloro-D-phenylalanine, 2-chloro-L-phenylalanine, 2-cyano-D-phenylalanine, 2-cyano-L-phenylalanine, 2-fluoro-D-phenylalanine, 2-fluoro-L-phenylalanine, 2-methyl-D-phenylalanine, 2-methyl-L-phenylalanine, 2-nitro-D-phenylalanine, 2-nitro-L-phenylalanine, 2,4,5-trihydroxy-phenylalanine, 3,4,5-trifluoro-D-phenylalanine, 3,4,5-trifluoro-L-phenylalanine, 3,4-dichloro-D-phenylalanine, 3,4-dichloro-L-phenylalanine, 3,4-difluoro-D-phenylalanine, 3,4-difluoro-L-phenylalanine, 3,4-dihydroxy-L-phenylalanine, 3,4-dimethoxy-L-phenylalanine, 3,5,3′-triiodo-L-thyronine, 3,5-diiodo-D-tyrosine, 3,5-diiodo-L-tyrosine, 3,5-diiodo-L-thyronine, 3-(trifluoromethyl)-D-phenylalanine, 3-(trifluoromethyl)-L-phenylalanine, 3-amino-L-tyrosine, 3-bromo-D-phenylalanine, 3-bromo-L-phenylalanine, 3-chloro-D-phenylalanine, 3-chloro-L-phenylalanine, 3-chloro-L-tyrosine, 3-cyano-D-phenylalanine, 3-cyano-L-phenylalanine, 3-fluoro-D-phenylalanine, 3-fluoro-L-phenylalanine, 3-fluoro-tyrosine, 3-iodo-D-phenylalanine, 3-iodo-L-phenylalanine, 3-iodo-L-tyrosine, 3-methoxy-L-tyrosine, 3-methyl-D-phenylalanine, 3-methyl-L-phenylalanine, 3-nitro-D-phenylalanine, 3-nitro-L-phenylalanine, 3-nitro-L-tyrosine, 4-(trifluoromethyl)-D-phenylalanine, 4-(trifluoromethyl)-L-phenylalanine, 4-amino-D-phenylalanine, 4-amino-L-phenylalanine, 4-benzoyl-D-phenylalanine, 4-benzoyl-L-phenylalanine, 4-bis(2-chloroethyl)amino-L-phenylalanine, 4-bromo-D-phenylalanine, 4-bromo-L-phenylalanine, 4-chloro-D-phenylalanine, 4-chloro-L-phenylalanine, 4-cyano-D-phenylalanine, 4-cyano-L-phenylalanine, 4-fluoro-D-phenylalanine, 4-fluoro-L-phenylalanine, 4-iodo-D-phenylalanine, 4-iodo-L-phenylalanine, homophenylalanine, thyroxine, 3,3-diphenylalanine, thyronine, ethyl-tyrosine, and methyl-tyrosine.

Exemplary amino acid analogs of proline include, but are not limited to, 3,4-dehydro-proline, 4-fluoro-proline, cis-4-hydroxy-proline, thiazolidine-2-carboxylic acid, and trans-4-fluoro-proline.

Exemplary amino acid analogs of serine and threonine include, but are not limited to, 3-amino-2-hydroxy-5-methylhexanoic acid, 2-amino-3-hydroxy-4-methylpentanoic acid, 2-amino-3-ethoxybutanoic acid, 2-amino-3-methoxybutanoic acid, 4-amino-3-hydroxy-6-methylheptanoic acid, 2-amino-3-benzyloxypropionic acid, 2-amino-3-benzyloxypropionic acid, 2-amino-3-ethoxypropionic acid, 4-amino-3-hydroxybutanoic acid, and α-methylserine.

Exemplary amino acid analogs of tryptophan include, but are not limited to, α-methyl-tryptophan, β-(3-benzothienyl)-D-alanine, β-(3-benzothienyl)-L-alanine, 1-methyl-tryptophan, 4-methyl-tryptophan, 5-benzyloxy-tryptophan, 5-bromo-tryptophan, 5-chloro-tryptophan, 5-fluoro-tryptophan, 5-hydroxy-tryptophan, 5-hydroxy-L-tryptophan, 5-methoxy-tryptophan, 5-methoxy-L-tryptophan, 5-methyl-tryptophan, 6-bromo-tryptophan, 6-chloro-D-tryptophan, 6-chloro-tryptophan, 6-fluoro-tryptophan, 6-methyl-tryptophan, 7-benzyloxy-tryptophan, 7-bromo-tryptophan, 7-methyl-tryptophan, D-1,2,3,4-tetrahydro-norharman-3-carboxylic acid, 6-methoxy-1,2,3,4-tetrahydronorharman-1-carboxylic acid, 7-azatryptophan, L-1,2,3,4-tetrahydro-norharman-3-carboxylic acid, 5-methoxy-2-methyl-tryptophan, and 6-chloro-L-tryptophan.

In some instances, the modification comprises a mutation to a hydrophobic or nonpolar amino acid residue. In some cases, a hydrophobic or nonpolar amino acid includes small hydrophobic amino acids and large hydrophobic amino acids. Exemplary small hydrophobic amino acids include glycine, alanine, proline, and analogs thereof. Exemplary large hydrophobic amino acids include valine, leucine, isoleucine, phenylalanine, methionine, tryptophan, and analogs thereof. In some instances, a neurotrophic agent described herein comprises a mutation to a small hydrophobic amino acid (e.g., a mutation to glycine, alanine, proline, and analogs thereof). In some instances, a neurotrophic agent described herein comprises a mutation to a large hydrophobic amino acid (e.g., a mutation to valine, leucine, isoleucine, phenylalanine, methionine, tryptophan, and analogs thereof).

In some instances, the modification comprises a mutation to a polar amino acid residue. In some cases, the polar amino acid comprises serine, threonine, asparagine, glutamine, cysteine, tyrosine, and analogs thereof. In some instances, a neurotrophic agent described herein comprises a mutation to a polar amino acid residue (e.g., a mutation to serine, threonine, asparagine, glutamine, cysteine, tyrosine, and analogs thereof).

In additional instances, the modification comprises a mutation to a charged amino acid residue. In some cases, charged amino acids include lysine, arginine, histidine, aspartate, glutamate, or analog thereof. In some instances, a neurotrophic agent described herein comprises a mutation to a charged amino acid residue (e.g., a mutation to lysine, arginine, histidine, aspartate, glutamate, or analog thereof).

In some embodiments, a neurotrophic agent described herein comprises a modification to a non-essential amino acid. In some instances, a non-essential amino acid residue is a residue that is altered from the wild-type sequence of a polypeptide without abolishing or substantially altering its essential biological or biochemical activity (e.g., receptor binding or activation). In some cases, a neurotrophic agent provided herein comprises an essential amino acid. In some cases, an essential amino acid residue is a residue that, when altered from the wild-type sequence of the polypeptide, results in abolishing or substantially abolishing the polypeptide's essential biological or biochemical activity.

In some embodiments, a neurotrophic agent described herein comprises a conservative amino acid substitution. In some cases, a conservative amino acid substitution is one in which the amino acid residue is replaced with an amino acid residue having a similar side chain. Families of amino acid residues having similar side chains have been defined in the art. These families include, for example, amino acids with basic side chains (e.g., K, R, H), acidic side chains (e.g., D, E), uncharged polar side chains (e.g., G, N, Q, S, T, Y, C), nonpolar side chains (e.g., A, V, L, I, P, F, M, W), beta-branched side chains (e.g., T, V, I) and aromatic side chains (e.g., Y, F, W, H). Thus, a predicted nonessential amino acid residue in a polypeptide, for example, is replaced with another amino acid residue from the same side chain family. Other examples of acceptable substitutions include substitutions based on isosteric considerations (e.g., norleucine for methionine) or other properties (e.g., 2-thienylalanine for phenylalanine, or 6-Cl-tryptophan for tryptophan).

In some embodiments, a neurotrophic agent described herein comprises a semi-conservative amino acid substitution. In some embodiments, the semi-conservative amino acid substitution involves substitution between amino acids within a same side chain family or amino acids in which the side chains have similar steric confirmation.

In some embodiments, a neurotrophic agent described herein comprises a non-conservative amino acid substitution. In some embodiments, the non-conservative amino acid substitution involves substitution between amino acids within a different side chain family.

In some embodiments, a neurotrophic agent described herein comprises a modification that is introduced by mutagenesis. In some embodiments, the mutagenesis is random. In some embodiments, mutagenesis is non-random. An example of non-random mutagenesis is site-directed mutagenesis. In some embodiments, the site-directed mutagenesis utilizes cassette mutagenesis, PCR-site-directed mutagenesis, whole plasmid mutagenesis, Kunkel's method, or in vivo site-directed mutagenesis method. In some instances, the mutagenesis is alanine screening mutagenesis.

In some embodiments, the neurotrophic agents described herein are chemically modified analogs of naturally occurring neurotrophic agents. Exemplary chemical modifications include, but are not limited to, phosphorylation or sulfurylation at serine, threonine, or tyrosine residues; by incorporating unnatural amino acids; by incorporating heavy amino acids; by incorporating D-amino acids; by biotinylation; by cyclisations; by acylation; by dimethylation; by amidation; by derivatization; by conjugation to carrier proteins; or by branching of peptide.

### Methods of Neurotrophic Agent Production

In some embodiments, one or more of the neurotropic agents described herein are produced, for example, in a host cell system or a cell-free system. In some embodiments, one or more of the neurotropic agents described herein are produced recombinantly through a host cell system. In some instances, the host cell is a eukaryotic cell (e.g., mammalian cell, insect cell, yeast cell, or plant cell) or a prokaryotic cell (e.g., gram-positive bacterium or a gram-negative bacterium).

In some embodiments, a eukaryotic host cell is a mammalian host cell. In some cases, a mammalian host cell is a stable cell line, or a cell line that has incorporated a genetic material of interest into its own genome and has the capability to express the product of the genetic material after many generations of cell division. In other cases, a mammalian host cell is a transient cell line, or a cell line that has not incorporated a genetic material of interest into its own genome and does not have the capability to express the product of the genetic material after many generations of cell division.

Exemplary mammalian host cells include 293T cell line, 293A cell line, 293FT cell line, 293F cells, 293 H cells, A549 cells, MDCK cells, CHO DG44 cells, CHO-S cells, CHO-K1 cells, Expi293FTM cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some embodiments, a eukaryotic host cell is an insect host cell. Exemplary insect host cell include Drosophila S2 cells, Sf9 cells, Sf21 cells, High Five™ cells, and expresSF+® cells.

In some embodiments, a eukaryotic host cell is a yeast host cell. Exemplary yeast host cells include Pichia pastoris yeast strains such as GS115, KM71H, SMD1168, SMD1168H, X-33, and Saccharomyces cerevisiae yeast strain such as INVSc1.

In some embodiments, a eukaryotic host cell is a plant host cell. In some instances, the plant cells comprise a cell from algae. Exemplary plant cell lines include strains from Chlamydomonas reinhardtii 137c or Synechococcus elongatus PPC 7942.

In some embodiments, a host cell is a prokaryotic host cell. Exemplary prokaryotic host cells include BL21, Mach1™, DH10B™, TOP10, DH5α, DH10Bac™, OmniMax™, MegaX™, DH12S™, INV110, IOP10F®, INVαF, TOP10/P3, ccdB Survival, PIR1, PIR2, Stb12™, Stb13™, or Stb14™.

In some instances, suitable polynucleic acid molecules or vectors for the production of a neurotropic agent described herein include any suitable vectors derived from either a eukaryotic or prokaryotic sources. Exemplary polynucleic acid molecules or vectors include vectors from bacteria (e.g., E. coli), insects, yeast (e.g., Pichia pastoris), algae, or mammalian source. Bacterial vectors include, for example, pACYC177, pASK75, pBAD vector series, pBADM vector series, pET vector series, pE™ vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Insect vectors include, for example, pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1 or pPolh-MAT2.

Yeast vectors include, for example, Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichi pastoris vector, pGAPZA, B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Algae vectors include, for example, pChlamy-4 vector or MCS vector.

Mammalian vectors include, for example, transient expression vectors or stable expression vectors. Exemplary mammalian transient expression vectors include p3xFLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3xFLAG-CMV 7.1, pFLAG-CMV 20, p3xFLAG-Myc-CMV 24, pCMV-FLAG-MAT1, pCMV-FLAG-MAT2, pBICEP-CMV 3, or pBICEP-CMV 4. Exemplary mammalian stable expression vectors include pFLAG-CMV 3, p3xFLAG-CMV 9, p3xFLAG-CMV 13, pFLAG-Myc-CMV 21, p3xFLAG-Myc-CMV 25, pFLAG-CMV 4, p3xFLAG-CMV 10, p3xFLAG-CMV 14, pFLAG-Myc-CMV 22, p3xFLAG-Myc-CMV 26, pBICEP-CMV 1, or pBICEP-CMV 2.

In some instances, a cell-free system is used for the production of a neurotrophic agent described herein. In some cases, a cell-free system comprises a mixture of cytoplasmic and/or nuclear components from a cell and is suitable for in vitro nucleic acid synthesis. In some instances, a cell-free system utilizes prokaryotic cell components. In other instances, a cell-free system utilizes eukaryotic cell components. Nucleic acid synthesis is obtained in a cell-free system based on, for example, Drosophila cell, Xenopus egg, or HeLa cells. Exemplary cell-free systems include E. coli S30 Extract system, E. coli T7 S30 system, or PURExpress®.

In some embodiments, one or more neurotropic agents described herein are chemically synthesized. Exemplary synthesis techniques include, for example, solid phase technique developed by R. B. Merrified, which permits the peptide to be built residue by residue from the carboxyl terminal amino acid to the amino terminal amino acid either manually or with an automated, commercially available synthesizer, and techniques described in Stewart, J. M. et al., Solid Phase Peptide Synthesis (Pierce Chemical Co., 2d ed., 1984), and Bodanszky, M. et al., The Practice of Peptide Synthesis (Springer-Verlag, 1984).

### Viral Vectors

In some embodiments, a polynucleic acid polymer that encodes a neurotrophic agent is expressed in a viral vector. In some instances, the neurotrophic agent is at least one of NGF, NT-3, NT-4, NT-5, BDNF, and PNT. In some instances, the neurotrophic agent is a chimeric neurotrophin. In some embodiments, the viral vector comprises a polynucleic acid polymer encoding a neurotrophic agent which comprises about 50%, 55%, 60%, 65%, 70%, 75%, 80%, 85%, 90%, 95%, 96%, 97%, 98%, 99%, or 100% sequence identity to the amino acid sequence set forth in SEQ ID NO: 8-30. In some embodiments, the viral vector comprises a polynucleic acid polymer encoding a neurotrophic agent which comprises about 70%, 80%, 85%, 90%, 91%, 92%, 93%, 94%, 95%, 96%, 97%, 98%, or 99% sequence identity to the amino acid sequence set forth in SEQ ID NO: 8-30. In some instances, the viral vector comprises a polynucleic acid polymer encoding a neurotrophic agent which comprises a sequence set forth in SEQ ID NO: 8-30.

In some embodiments, the viral vector is obtained from any virus, such as a DNA or an RNA virus. In some embodiments, a DNA virus is a single-stranded (ss) DNA virus, a double-stranded (ds) DNA virus, or a DNA virus that contains both ss and ds DNA regions. In some embodiments, an RNA virus is a single-stranded (ss) RNA virus or a double-stranded (ds) RNA virus. In some embodiments, a ssRNA virus is further classified into a positive-sense RNA virus or a negative-sense RNA virus.

In some instances, the viral vector is obtained from a dsDNA virus of the family: Myoviridae, Podoviridae, Siphoviridae, Alloherpesviridae, Herpesviridae, Malacoherpesviridae, Lipothrixviridae, Rudiviridae, Adenoviridae, Ampullaviridae, Ascoviridae, Asfaviridae, Baculoviridae, Bicaudaviridae, Clavaviridae, Corticoviridae, Fuselloviridae, Globuloviridae, Guttaviridae, Hytrosaviridae, Iridoviridae, Marseilleviridae, Mimiviridae, Nimaviridae, Pandoraviridae, Papillomaviridae, Phycodnaviridae, Plasmaviridae, Polydnaviruses, Polyomaviridae, Poxviridae, Sphaerolipoviridae, and Tectiviridae.

In some cases, the viral vector is obtained from a ssDNA virus of the family: Anelloviridae, Bacillariodnaviridae, Bidnaviridae, Circoviridae, Geminiviridae, Inoviridae, Microviridae, Nanoviridae, Parvoviridae, and Spiraviridae.

In some embodiments, the viral vector is obtained from a DNA virus that contains both ss and ds DNA regions. In some cases, the DNA virus is from the group pleolipoviruses. In some cases, the pleolipoviruses include Haloarcula hispanica pleomorphic virus 1, Halogeometricum pleomorphic virus 1, Halorubrum pleomorphic virus 1, Halorubrum pleomorphic virus 2, Halorubrum pleomorphic virus 3, and Halorubrum pleomorphic virus 6.

In some cases, the viral vector is obtained from a dsRNA virus of the family: Birnaviridae, Chrysoviridae, Cystoviridae, Endornaviridae, Hypoviridae, Megavirnaviridae, Partitiviridae, Picobirnaviridae, Reoviridae, Rotavirus, and Totiviridae.

In some instances, the viral vector is obtained from a positive-sense ssRNA virus of the family: Alphaflexiviridae, Alphatetraviridae, Alvernaviridae, Arteriviridae, Astroviridae, Barnaviridae, Betaflexiviridae, Bromoviridae, Caliciviridae, Carmotetraviridae, Closteroviridae, Coronaviridae, Dicistroviridae, Flaviviridae, Gammaflexiviridae, Iflaviridae, Leviviridae, Luteoviridae, Marnaviridae, Mesoniviridae, Narnaviridae, Nodaviridae, Permutotetraviridae, Picornaviridae, Potyviridae, Roniviridae, Secoviridae, Togaviridae, Tombusviridae, Tymoviridae, and Virgaviridae.

In some cases, the viral vector is obtained from a negative-sense ssRNA virus of the family: Bornaviridae, Filoviridae, Paramyxoviridae, Rhabdoviridae, Nyamiviridae, Arenaviridae, Bunyaviridae, Ophioviridae, and Orthomyxoviridae.

In some instances, the viral vector is obtained from oncolytic DNA viruses that comprise capsid symmetry that is icosahedral or complex. In some cases, icosahedral oncolytic DNA viruses are naked or comprise an envelope. Exemplary families of oncolytic DNA viruses include the Adenoviridae (for example, Adenovirus, having a genome size of 36-38 kb), Herpesviridae (for example, HSV1, having a genome size of 120-200 kb), and Poxviridae (for example, Vaccinia virus and myxoma virus, having a genome size of 130-280 kb).

In some cases, the viral vector is obtained from oncolytic RNA viruses include those having icosahedral or helical capsid symmetry. In some cases, icosahedral oncolytic viruses are naked without envelope and include Reoviridae (for example, Reovirus, having a genome of 22-27 kb) and Picornaviridae (for example, Poliovirus, having a genome size of 7.2-8.4 kb). In other cases, helical oncolytic RNA viruses are enveloped and include Rhabdoviridae (for example, VSV, having genome size of 13-16 kb) and Paramyxoviridae (for example MV and NDV, having genome sizes of 16-20 kb).

Exemplary viral vectors include, but are not limited to, retroviral vectors, lentiviral vectors, adenoviral vectors, adeno-associated viral vectors, alphaviral vectors, herpes simplex virus vectors, vaccinia viral vectors, or chimeric viral vectors. In some embodiments, the viral vector is a lentiviral vector. In some embodiments, the lentiviral vector is pLKO.1 vector.

In some instances, a virus comprising a polynucleic acid polymer that encodes for a neurotrophic agent is generated using methods well known in the art. In some instances, the methods involve one or more transfection steps and one or more infection steps. In some instances, a cell line such as a mammalian cell line, an insect cell line, or a plant cell line is infected with a virus to produce one or more viruses. Exemplary mammalian cell lines include: 293A cell line, 293FT cell line, 293F cells, 293 H cells, CHO DG44 cells, CHO-S cells, CHO-K1 cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, T-REx™-HeLa cell line, 3T6, A549, A9, AtT-20, BALB/3T3, BHK-21, BHL-100, BT, Caco-2, Chang, Clone 9, Clone M-3, COS-1, COS-3, COS-7, CRFK, CV-1, D-17, Daudi, GH1, GH3, H9, HaK, HCT-15, HEp-2, HL-60, HT-1080, HT-29, HUVEC, I-10, IM-9, JEG-2, Jensen, K-562, KB, KG-1, L2, LLC-WRC 256, McCoy, MCF7, VERO, WI-38, WISH, XC, or Y-1. Exemplary insect cell lines include Drosophila S2 cells, Sf9 cells, Sf21 cells, High Five™ cells, or expresSF+® cells. Exemplary plant cell lines include algae cells such as for example Phaeocystis pouchetii.

In some embodiments, the vector comprising a polynucleic acid polymer that encodes for a neurotrophic agent described herein is delivered through electroporation, chemical method, microinjection, gene gun, impalefaction, hydrodynamics-based delivery, continuous infusion, or sonication. In some embodiments, the chemical method is lipofection. In some cases, the method is infection, or adsorption or transcytosis.

In some embodiments, electroporation is a technique in which an electric field is applied to cells to increase the permeability of the cell membrane, allowing for the introduction of chemicals, drugs, or DNA into the cell.

In some embodiments, chemical method is a method of transfection that uses carrier molecules to overcome the cell-membrane barrier. In some instances, the chemical method is lipofection whereby genetic material is injected into a cell using liposomes.

In some embodiments, microinjection is the injection of genetic material into animal cells, tissues, or embryos via a needle.

In some embodiments, gene gun is a device that injects cells with genetic information by shooting them with elemental particle of a heavy metal coated with plasmid DNA.

In some embodiments, impalefaction is a method of gene delivery using nanomaterials.

In some embodiments, hydrodynamics-based delivery is the rapid injection of a relatively large volume of solution into a blood vessel to enhance the permeability to allow for the delivery of substance into cells. In some instances, the solution contains proteins, oligo nucleotides, DNA, RNA, or small molecules.

In some embodiments, continuous infusion is the uninterrupted administration of drugs, fluids or nutrients into a blood vessel.

In some embodiments, sonication is applying sound energy to agitate particles in a sample for purposes such as but not limited to disrupting or deactivating a biological material or fragmenting molecules of DNA.

In some embodiments, the neurotrophic agent is delivered as an injection, such as an intramuscular, intrathecal, intravitreal, intraconjunctival, intravenous, or subcutaneous injection, without the need of a viral delivery method or non-viral delivery methods such as electroporation, chemical method, microinjection, gene gun, impalefaction, hydrodynamics-based delivery, continuous infusion, or sonication. In some instances, the vector as described above is delivered as an injection, such as an intramuscular, intrathecal, intravitreal, intraconjunctival, intravenous, or subcutaneous injection, without the need of a viral delivery method or non-viral delivery methods such as electroporation, chemical method, microinjection, gene gun, impalefaction, hydrodynamics-based delivery, continuous infusion, or sonication.

In some embodiments, the neurotrophic agent or the vector comprising a polynucleic acid polymer that encodes for a neurotrophic agent described above further comprises a delivery vehicle. In some instances, the delivery vehicle comprises a lipid-based nanoparticle; a cationic cell penetrating peptide (CPP); a linear or branched cationic polymer; or a bioconjugate, such as cholesterol, bile acid, lipid, peptide, polymer, protein, or an aptamer, which is conjugated to the nucleic acid polymer or polypeptide described herein for intracellular delivery. In some instances, additional delivery vehicles comprise glycopolymer, carbohydrate polymer, or lipid polymers such as cationic lipids or cationic lipid polymers.

### Diseases

Disclosed herein, in certain embodiments, is a method of treating a non-otic disease or condition associated with an elevated expression level or activity of a Trk receptor (e.g., TrkA, TrkB, and TrkC). In some embodiments, the non-otic disease or condition is associated with a reduced expression level or activity of a Trk receptor. In other embodiments, the non-otic disease or condition is associated with an elevated expression level or activity of a p75^{NTR} receptor. In some instances, the method comprises administering to a patient having a non-otic disease or condition a therapeutic amount of a pharmaceutical composition described herein. In some instances, also described herein, include a method of preventing a non-otic disease or condition associated with an elevated expression level or activity of a Trk receptor. In some embodiments, the non-otic disease or condition is associated with a reduced expression level or activity of a Trk receptor. In some instances, the method comprises administering to a patient having a non-otic disease or condition a therapeutic amount of a pharmaceutical composition described herein.

In some embodiments, the non-otic diseases or conditions comprise a neurodegenerative disease or condition, or a symptomatic or pre-symptomatic condition with loss or alterations of synapses. In some instances, the non-otic diseases or conditions comprise neurodegenerative disorders. In some embodiments, the neurodegenerative disease or condition comprises polyglutamine expansion disorder, fragile X syndrome, fragile XE mental retardation, Rett syndrome, Friedreich's ataxia, myotonic dystrophy, spinocerebellar ataxia type 8, spinocerebellar ataxia type 12, Alexander disease, Alper's disease, Alzheimer's disease, amyotrophic lateral sclerosis (ALS), ataxia telangiectasia, Batten disease (Spielmeyer-Vogt-Sjogren-Batten disease), Canavan disease, Cockayne syndrome, corticobasal degeneration, Creutzfeldt-Jakob disease, glaucoma, ischemia stroke, Krabbe disease, Lewy body dementia, dementia, multiple sclerosis, multiple system atrophy, Parkinson's disease, Pelizaeus-Merzbacher disease, Pick's disease, primary lateral sclerosis, progressive muscular atrophy, progressive bulbar palsy, pseudobulbar palsy, retinitis pigmentosa, Refsum's disease, Sandhoff disease, Schilder's disease, spinal cord injury (SCI), traumatic brain injury, spinal muscular atrophy (SMA), Steele-Richardson-Olszewski disease, Tabes dorsalis, neuropathy (e.g., diabetic, AIDS), and pain (e.g, osteoarthritis, rheumatoid, cancer).

In some embodiments, the polyglutamine repeat disease comprises Huntington's disease (HD), dentatorubropallidoluysian atrophy, Kennedy's disease (also referred to as spinobulbar muscular atrophy), or a spinocerebellar ataxia selected from the group consisting of type 1, type 2, type 3 (Machado-Joseph disease), type 6, type 7, and type 17.

In some embodiments, the non-otic disease or condition comprises amyotrophic lateral sclerosis (ALS), spinal muscular atrophy (SMA), glaucoma, spinal cord injury (SCI), ulcers (e.g., corneal, viral infection), or retinitis pigmentosa. In some embodiments, the non-otic disease or condition is amyotrophic lateral sclerosis (ALS). In some embodiments, the non-otic disease or condition is spinal muscular atrophy (SMA). In some embodiments, the non-otic disease or condition is glaucoma. In some embodiments, the non-otic disease or condition is spinal cord injury (SCI). In some embodiments, the non-otic disease or condition is retinitis pigmentosa.

In some instances, the non-otic diseases or conditions comprise eye disorders or diseases. Non-limiting eye disorders or diseases include retinitis pigmentosa, macular degeneration, glaucoma, diabetic retinopathy, corneal epithelial damage, Goblet cell defect, and dry eye.

In some instances, the non-otic diseases or conditions comprise psychiatric disorders. Exemplary psychiatric disorders comprise depression, psychosis, schizophrenia, narcolepsy, suicide tendency, autism, addiction, synaptopathy, or eating disorder (e.g., compulsive overeating or obesity).

In some instances, the non-otic diseases or conditions comprise cancer. Exemplary diseases caused by Trk signaling upregulation include, but are not limited to, multiple myeloma, neuroblastoma, gastric cancer, head and neck squamous cell carcinoma, choriocarcinoma, non-small cell lung cancer, lung adenocarcinoma, pulmonary fibrosis, and alveolar cell hyperplasia.

In some instances, the non-otic diseases or conditions are a result of an elevated level of expression or activity of a Trk receptor. In some instances, the non-otic diseases or conditions are a result of an elevated level of expression or activity of a neurotrophin (e.g. NGF, NT-3, NT-4, NT-5, and BDNF). In some cases, elevated level of expression or activity of the neurotrophin results in a neurodegenerative disease or disorder. For example, it has been shown that NGF expression is increased in Alzheimer's disease. In some instances, increased levels of BDNF, NT-3, NT-4, and NT-5 results in ALS.

In some instances, the non-otic diseases or conditions are a result of a reduced level of expression or activity of a Trk receptor. In some instances, the non-otic diseases or conditions are a result of a reduced level of expression or activity of a neurotrophin (e.g. NGF, NT-3, NT-4, NT-5, and BDNF). For example, it has been shown that reduced expression of BDNF and TrkB is linked to Alzheimer's disease. In some instances, reduced expression of BDNF results in death of striatal neurons in Huntington's disease.

### Pharmaceutical Composition/Formulations

In some embodiments, pharmaceutical compositions or formulations described herein are administered to a subject by multiple administration routes, including, but not limited to, parenteral (e.g., intravenous, intrathecal, intravitreal, intraconjunctival, cutaneous, subcutaneous, intramuscular), oral, intranasal, intraocular, buccal, topical, rectal, or transdermal administration routes. In some instances, the pharmaceutical composition described herein is formulated for parenteral (e.g., intravenous, intrathecal, intravitreal, intraconjunctival, cutaneous, subcutaneous, intramuscular) administration. In other instances, the pharmaceutical composition described herein is formulated for oral administration. In still other instances, the pharmaceutical composition described herein is formulated for intranasal administration. In still other instances, the pharmaceutical composition described herein is formulated for intraocular administration. In some instances, the pharmaceutical composition is formulated for topical administration. In some instances, the pharmaceutical composition described herein is formulated as an eye-drop.

In some embodiments, pharmaceutical formulation described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In some embodiments, pharmaceutical formulations described herein include a carrier or carrier materials which include any commonly used excipients in pharmaceutics and are selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

In some embodiments, pharmaceutical formulations include dispersing agents and/or viscosity modulating agents which include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®, and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K 100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide), poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, polyvinylpyrrolidone K30, polyvinylpyrrolidone/vinyl acetate copolymer (S-630), polyethylene glycol (e.g., the polyethylene glycol has a molecular weight of from about 300 to about 6000, or from about 3350 to about 4000, or from about 7000 to about 5400), sodium carboxymethylcellulose, methylcellulose, polysorbate-80, sodium alginate, gums (such as, e.g., gum tragacanth and gum acacia), guar gum, xanthans (including xanthan gum), sugars, cellulosics (such as, e.g., sodium carboxymethylcellulose, methylcellulose, sodium carboxymethylcellulose), polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose are also used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol, and isopropyl myristate.

In some embodiments, pharmaceutical formulations include pH adjusting agents or buffering agents which include acids such as acetic, boric, citric, lactic, phosphoric, and hydrochloric acids; bases such as sodium hydroxide, sodium phosphate, sodium borate, sodium citrate, sodium acetate, sodium lactate, and tris-hydroxymethylaminomethane; and buffers such as citrate/dextrose, sodium bicarbonate, and ammonium chloride. Such acids, bases, and buffers are included in an amount required to maintain pH of the composition in an acceptable range.

In some embodiments, pharmaceutical formulations also include one or more salts in an amount required to bring osmolality of the composition into an acceptable range. Such salts include those such as having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some embodiments, pharmaceutical formulations further include diluents, which are also used to stabilize compounds because they provide a more stable environment. Salts dissolved in buffered solutions (which also provide pH control or maintenance) are utilized as diluents in the art, including, but not limited to, a phosphate buffered saline solution. In certain instances, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®, dibasic calcium phosphate, dicalcium phosphate dihydrate; tricalcium phosphate, calcium phosphate, anhydrous lactose, spray-dried lactose, pregelatinized starch, compressible sugar (such as DiPac®, Amstar), mannitol, hydroxypropylmethylcellulose, hydroxypropylmethylcellulose acetate stearate, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like.

In some embodiments, pharmaceutical formulations include disintegration agents or disintegrants to facilitate the breakup or disintegration of a substance. The term “disintegrate” includes both the dissolution and dispersion of the dosage form when contacted with gastrointestinal fluid. Examples of disintegration agents include a starch, e.g., a natural starch such as corn starch or potato starch; a pregelatinized starch such as National 1551 or Amijel®; sodium starch glycolate, such as Promogel® or Explotab®; a cellulose such as a wood product, methylcrystalline cellulose (e.g., Avicel®, Avicel® PH101, AvicerPH102, Avicel® PH105, Elcema® P100, Emcocel®, Vivacel®, Ming Tia®, and)Solka-Floc®, methylcellulose, croscarmellose, or a cross-linked cellulose (such as cross-linked sodium carboxymethylcellulose (Ac-Di-Sol®), cross-linked carboxymethylcellulose, or cross-linked croscarmellose); a cross-linked starch, such as sodium starch glycolate; a cross-linked polymer, such as crospovidone; a cross-linked polyvinylpyrrolidone; alginate, such as alginic acid; a salt of alginic acid, such as sodium alginate; a clay, such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination with starch; and the like.

In some embodiments, pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

In some embodiments, pharmaceutical formulations include flavoring agents and/or sweeteners, such as, for example, acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate) (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

Lubricants and glidants also included in the pharmaceutical formulations described herein, for example, include those that prevent, reduce, or inhibit adhesion or friction of materials. Exemplary lubricants include, e.g., stearic acid; calcium hydroxide; talc, sodium stearyl fumerate; a hydrocarbon such as mineral oil; hydrogenated vegetable oil such as hydrogenated soybean oil)(Sterotex®; higher fatty acids and their alkali-metal and alkaline earth metal salts, such as aluminum, calcium, magnesium, zinc; stearic acid; sodium stearates; glycerol; talc; waxes; Stearowet; boric acid; sodium benzoate; sodium acetate; sodium chloride; leucine; a polyethylene glycol (e.g., PEG-4000); a methoxypolyethylene glycol, such as Carbowax™; sodium oleate; sodium benzoate; glyceryl behenate; polyethylene glycol; magnesium or sodium lauryl sulfate; colloidal silica, such as Syloid™; Cab-O-Sil®; a starch, such as corn starch; silicone oil; a surfactant, and the like.

Plasticizers include compounds used to soften the microencapsulation material or film coatings to make them less brittle. Suitable plasticizers include, e.g., polyethylene glycols, such as PEG 300, PEG 400, PEG 600, PEG 1450, PEG 3350, and PEG 800; stearic acid; propylene glycol; oleic acid; triethyl cellulose; and triacetin. Plasticizers also function as dispersing agents or wetting agents.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, dimethyl isosorbide, and the like.

Stabilizers include compounds such as any antioxidation agents, buffers, acids, preservatives, and the like.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630); polyethylene glycol, e.g., the polyethylene glycol having a molecular weight of from about 300 to about 6000, or from about 3350 to about 4000, or from about 7000 to about 5400; sodium carboxymethylcellulose; methylcellulose; hydroxypropylmethylcellulose; hydroxymethylcellulose acetate stearate; polysorb ate-80; hydroxyethylcellulose; sodium alginate; gums, such as, e.g., gum tragacanth and gum acacia; guar gum; xanthans, including xanthan gum; sugars; cellulosics, such as, e.g., sodium carboxymethylcellulose, methylcellulose, sodium carboxymethylcellulose, hydroxypropylmethylcellulose, hydroxyethylcellulose; polysorbate-80; sodium alginate; polyethoxylated sorbitan monolaurate; polyethoxylated sorbitan monolaurate; povidone; and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide (e.g., Pluronic® (BASF)), and the like. Additional surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. Sometimes, surfactants are included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans, and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts, and the like.

Injectable Formulations

Formulations suitable for intramuscular, intrathecal, intravitreal, intraconjunctival, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil), and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain additives such as preserving, wetting, emulsifying, and dispensing agents. Prevention of the growth of microorganisms is ensured by various antibacterial and antifungal agents, such as parabens, chlorobutanol, phenol, sorbic acid, and the like. It also is desirable to include isotonic agents, such as sugars, sodium chloride, and the like. Prolonged absorption of the injectable pharmaceutical form is brought about by the use of agents delaying absorption, such as aluminum monostearate and gelatin.

For intravenous injections, compounds described herein are formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are generally known in the art.

In some instances, parenteral injections involve bolus injection or continuous infusion. Formulations for injection are presented in unit dosage form, e.g., in ampoules or in multi-dose containers, with an added preservative. The pharmaceutical composition described herein is in a form suitable for parenteral injection as a sterile suspension, solution or emulsion in oily or aqueous vehicle, and contains formulatory agents such as suspending, stabilizing, and/or dispersing agents. Pharmaceutical formulations for parenteral administration include aqueous solutions of the active compounds in water-soluble form. Additionally, suspensions of the active compounds are prepared as appropriate oily injection suspensions. Suitable lipophilic solvents or vehicles include fatty oils, such as sesame oil; synthetic fatty acid esters, such as ethyl oleate or triglycerides; or liposomes. Aqueous injection suspensions contain substances which increase the viscosity of the suspension, such as sodium carboxymethyl cellulose, sorbitol, or dextran. Optionally, the suspension also contains suitable stabilizers or agents which increase the solubility of the compounds to allow for the preparation of highly concentrated solutions. Alternatively, the active ingredient is in powder form for constitution with a suitable vehicle, e.g., sterile pyrogen-free water, before use.

Oral Formulations

Pharmaceutical preparations for oral use are obtained by mixing one or more solid excipients with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as polyvinylpyrrolidone (PVP or povidone) or calcium phosphate. If desired, disintegrating agents are added, such as the cross-linked croscarmellose sodium, polyvinylpyrrolidone, agar, or alginic acid, or a salt thereof such as sodium alginate.

Dragee cores are provided with suitable coatings. For this purpose, concentrated sugar solutions are used, which optionally contain gum arabic, talc, polyvinylpyrrolidone, carbopol gel, polyethylene glycol, titanium dioxide, lacquer solutions, and suitable organic solvents or solvent mixtures. Dyestuffs or pigments are added to the tablets or dragee coatings for identification or to characterize different combinations of active compound doses.

Solid dosage forms are in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder), a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other instances, the pharmaceutical formulation is in the form of a powder. In still other instances, the pharmaceutical formulation is in the form of a tablet, including but not limited to, a fast-melt tablet. Additionally, pharmaceutical formulations described herein are administered as a single capsule or in multiple capsule dosage form. In some cases, the pharmaceutical formulation is administered in two, three, or four, capsules or tablets.

In some embodiments, the pharmaceutical solid dosage forms include a composition described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington's Pharmaceutical Sciences, 20th Edition (2000).

In some embodiments, suitable carriers for use in the solid dosage forms include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol, and the like.

In some embodiments, suitable filling agents for use in the solid dosage forms include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

Binders impart cohesiveness to solid oral dosage form formulations; for powder filled capsule formulation, binders aid in plug formation that are filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose (e.g., Methocel), hydroxypropylmethylcellulose (e.g. Hypromellose USP Pharmacoat-603), hydroxypropylmethylcellulose acetate stearate (Aqoate HS-LF and HS), hydroxyethylcellulose, hydroxypropylcellulose (e.g., Klucel®), ethylcellulose (e.g., Ethocel®), microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar (such as sucrose (e.g., Dipac®), glucose, dextrose, molasses, mannitol, sorbitol, xylitol (e.g., Xylitab®), lactose), a natural or synthetic gum (such as acacia, tragacanth, ghatti gum, mucilage of isapol husks), starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like.

Suitable lubricants or glidants for use in the solid dosage forms include, but are not limited to, stearic acid; calcium hydroxide; talc; corn starch; sodium stearyl fumerate; alkali-metal and alkaline earth metal salts, such as aluminum, calcium, magnesium, zinc; stearic acid; sodium stearates, magnesium stearate, zinc stearate; waxes; Stearowet®; boric acid; sodium benzoate; sodium acetate; sodium chloride; leucine; a polyethylene glycol or a methoxypolyethylene glycol such as Carbowax™, PEG 4000, PEG 5000, PEG 6000, propylene glycol, sodium oleate; glyceryl behenate; glyceryl palmitostearate; glyceryl benzoate; magnesium or sodium lauryl sulfate; and the like.

Suitable diluents for use in the solid dosage forms include, but are not limited to, sugars (including lactose, sucrose, and dextrose), polysaccharides (including dextrates and maltodextrin), polyols (including mannitol, xylitol, and sorbitol), cyclodextrins, and the like.

Suitable wetting agents for use in the solid dosage forms include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS, and the like.

Suitable surfactants for use in the solid dosage forms include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms include, but are not limited to, polyvinylpyrrolidone, e.g., polyvinylpyrrolidone K12, polyvinylpyrrolidone K17, polyvinylpyrrolidone K25, or polyvinylpyrrolidone K30; polyethylene glycol, e.g., the polyethylene glycol having a molecular weight from about 300 to about 6000, from about 3350 to about 4000, or from about 7000 to about 5400; vinyl pyrrolidone/vinyl acetate copolymer (S630); sodium carboxymethylcellulose; methylcellulose; hydroxy-propylmethylcellulose; polysorbate-80; hydroxyethylcellulose; sodium alginate; gums, such as, e.g., gum tragacanth and gum acacia; guar gum; xanthans, including xanthan gum; sugars; cellulosics, such as, e.g., sodium carboxymethylcellulose, methylcellulose, sodium carboxymethylcellulose, hydroxypropylmethylcellulose, hydroxyethylcellulose; polysorbate-80; sodium alginate; polyethoxylated sorbitan monolaurate; polyethoxylated sorbitan monolaurate; povidone; and the like.

Suitable antioxidants for use in the solid dosage forms include, for example, e.g., butylated hydroxytoluene (BHT), sodium ascorbate, and tocopherol.

Liquid formulation dosage forms for oral administration include aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2^{nd} Ed., pp. 754-757 (2002). In addition the liquid dosage forms include additives, such as: (a) disintegrating agents; (b) dispersing agents; (c) wetting agents; (d) at least one preservative; (e) viscosity enhancing agents; (f) at least one sweetening agent; and (g) at least one flavoring agent. In some embodiments, the aqueous dispersions further include a crystalline inhibitor.

In some embodiments, the aqueous suspensions and dispersions described herein remain in a homogenous state, as defined in The USP Pharmacists' Pharmacopeia (2005 edition, chapter 905), for at least 4 hours. The homogeneity should be determined by a sampling method consistent with regard to determining homogeneity of the entire composition. In one embodiment, an aqueous suspension is re-suspended into a homogenous suspension by physical agitation lasting less than 1 minute. In another aspect, an aqueous suspension is re-suspended into a homogenous suspension by physical agitation lasting less than 45 seconds. In yet another aspect, an aqueous suspension is re-suspended into a homogenous suspension by physical agitation lasting less than 30 seconds. In still another embodiment, no agitation is necessary to maintain a homogeneous aqueous dispersion.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, carrier materials such as binders, suspending agents, disintegration agents, filling agents, surfactants, solubilizers, stabilizers, lubricants, wetting agents, and diluents.

Exemplary microencapsulation materials useful for delaying the release of the formulations including compounds described herein, include, but are not limited to, hydroxypropyl cellulose ethers (HPC) such as Klucel® or Nisso HPC; low-substituted hydroxypropyl cellulose ethers (L-HPC); hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843; methylcellulose polymers such as Methocel®-A, hydroxypropylmethylcellulose acetate stearate Aqoat (HF-LS, HF-LG,HF-MS), and Metolose °; ethylcelluloses (EC) and mixtures thereof such as E461, Ethocel®, Aqualon®-EC, Surelease®; polyvinyl alcohol (PVA) such as Opadry AMB; hydroxyethylcelluloses such as Natrosol®; carboxymethylcelluloses and salts of carboxymethylcelluloses (CMC) such as Aqualon®-CMC; polyvinyl alcohol and polyethylene glycol co-polymers such as Kollicoat IR®; monoglycerides (Myverol); triglycerides (KLX); polyethylene glycols; modified food starch; acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D; cellulose acetate phthalate; sepifilms such as mixtures of HPMC and stearic acid; cyclodextrins; and mixtures of these materials.

Plasticizers, such as polyethylene glycols; e.g., PEG 300, PEG 400, PEG 600, PEG 1450, PEG 3350, and PEG 800; stearic acid; propylene glycol; oleic acid; and triacetin, are incorporated into the microencapsulation material. In other embodiments, the microencapsulating material useful for delaying the release of the pharmaceutical compositions is from the USP or the National Formulary (NF). In yet other embodiments, the microencapsulation material is Klucel. In still other embodiments, the microencapsulation material is methocel.

Microencapsulated compositions are formulated by methods known by one of ordinary skill in the art. Such known methods include, e.g., spray drying processes, spinning disk-solvent processes, hot melt processes, spray chilling methods, fluidized bed, electrostatic deposition, centrifugal extrusion, rotational suspension separation, polymerization at liquid-gas or solid-gas interface, pressure extrusion, or spraying solvent extraction bath. In addition to these, several chemical techniques, e.g., complex coacervation, solvent evaporation, polymer-polymer incompatibility, interfacial polymerization in liquid media, in situ polymerization, in-liquid drying, and desolvation in liquid media are also used. Furthermore, other methods such as roller compaction, extrusion/spheronization, coacervation, or nanoparticle coating are also used.

Intranasal Formulations

Intranasal formulations are known in the art and are described in, for example, U.S. Pat. Nos. 4,476,116 and 6,391,452. Formulations that include the compositions described herein, which are prepared according to the above described and other techniques well-known in the art are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these are found in Remington: The Science and Practice of Pharmacy, 21st edition, 2005, a standard reference in the field. The choice of suitable carriers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are also present. The nasal dosage form should be isotonic with nasal secretions.

For administration by inhalation described herein include aerosol, mist, or powder. Pharmaceutical compositions described herein are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit is determined by providing a valve to deliver a metered amount. Capsules and cartridges of, such as, by way of example only, gelatin for use in an inhaler or insufflator are formulated containing a powder mix of the compound described herein and a suitable powder base such as lactose or starch.

### Dosing and Treatment Regimens

In some embodiments, the pharmaceutical compositions described herein are administered for therapeutic applications. In some embodiments, the pharmaceutical compositions described herein are also administered as a maintenance therapy, for example for a patient in remission. In some embodiments, the pharmaceutical composition is administered once per day, twice per day, three times per day, or more. In some embodiments, the pharmaceutical composition is administered daily, every day, every alternate day, five days a week, once a week, every other week, two weeks per month, three weeks per month, once a month, twice a month, three times per month, or more. In some embodiments, the pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the patient's status does improve, upon the doctor's discretion the administration of the compounds is given continuously; alternatively, the dose of drug being administered is temporarily reduced or temporarily suspended for a certain length of time (i.e., a “drug holiday”). The length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, are reduced, as a function of the symptoms, to a level at which the improved disease, disorder or condition is retained. In some instances, patients, however, require intermittent treatment of a pharmaceutical composition described herein on a long-term basis upon any recurrence of symptoms.

In some embodiments, the amount of a given agent that corresponds to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four, or more sub-doses per day.

In some embodiments, the pharmaceutical composition described herein is in unit dosage forms suitable for single administration of precise dosages. In unit dosage form, the formulation is divided into unit doses containing appropriate quantities of one or more compound. In some embodiments, the unit dosage is in the form of a package containing discrete quantities of the formulation. Non-limiting examples are packaged tablets or capsules, and powders in vials or ampoules. Aqueous suspension compositions in some instances are packaged in single-dose non-reclosable containers. Alternatively, multiple-dose reclosable containers in some instances are used, in which case it is typical to include a preservative in the composition. By way of example only, formulations for parenteral injection are presented in unit dosage form, which include, but are not limited to, ampoules or in multi-dose containers, with an added preservative.

The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in humans. The dosage of such compounds lies preferably within a range of circulating concentrations that include the ED50 with minimal toxicity. The dosage varies within this range depending upon the dosage form employed and the route of administration utilized.

According to another aspect of the invention, there is provided a method of selecting a subject for treatment, comprising determining if the subject has a disease induced by defective protein expression caused by the intron retention in gene transcripts, wherein the subject is selected for treatment upon positive confirmation, and optionally treating the subject.

### Diagnostic Methods

In certain embodiments, also included are methods of stratifying an individual having a non-otic disease or condition for treatment with a pharmaceutical composition described herein and methods of optimizing the therapy of an individual receiving a pharmaceutical composition described herein for treatment of a non-otic disease or condition. In some instances, disclosed herein is a method of stratifying an individual having a non-otic disease or condition for treatment with a pharmaceutical composition described herein, comprising: determining the expression level of a Trk receptor; and administering to the individual a therapeutically effective amount of the pharmaceutical composition if there is an elevated or reduced expression level of the Trk receptor. In some instances, also disclosed herein is a method of optimizing the therapy of an individual receiving a pharmaceutical composition described herein for treatment of a non-otic disease or condition, comprising: determining the expression level of a Trk receptor; and modifying, discontinuing, or continuing the treatment based on the expression level of the Trk receptor.

Methods for determining the expression and/or activity of Trk receptor are well known in the art. In some embodiments, the expression levels are measured at either nucleic acid level or protein level, and by methods such as RT-PCR, Qt-PCR, microarray, Northern blot, ELISA, radioimmunoassay (RIA), electrochemiluminescence (ECL), Western blot, multiplexing technologies, or other similar methods. In some embodiments, activities of the Trk receptor are measured by methods such as co-immunoprecipitation, fluorescence spectroscopy, fluorescence resonance energy transfer (FRET), isothermal titration calorimetry (ITC), dynamic light scattering (DLS), surface plasmon resonance (SPR), or other similar methods.

In some embodiments, the expression of a Trk receptor is determined at the nucleic acid level. Nucleic acid-based techniques for assessing expression are well known in the art and include, for example, determining the level of biomarker mRNA in a biological sample. Many expression detection methods use isolated RNA. Any RNA isolation technique that does not select against the isolation of mRNA is utilized for the purification of RNA (see, e.g., Ausubel et al., ed. (1987-1999) Current Protocols in Molecular Biology (John Wiley & Sons, New York). Additionally, large numbers of tissue samples are readily processed using techniques well known to those of skill in the art, such as, for example, the single-step RNA isolation process disclosed in U.S. Pat. No. 4,843,155.

Thus, in some embodiments, the detection of a biomarker is assayed at the nucleic acid level using nucleic acid probes. The term “nucleic acid probe” refers to any molecule that is capable of selectively binding to a specifically intended target nucleic acid polymer, for example, a nucleotide transcript. Probes are synthesized by one of skill in the art or derived from appropriate biological preparations. Probes are specifically designed to be labeled, for example, with a radioactive label, a fluorescent label, an enzyme, a chemiluminescent tag, a colorimetric tag, or other labels or tags that are discussed above or that are known in the art. Examples of molecules that are utilized as probes include, but are not limited to, RNA and DNA.

For example, isolated mRNA are used in hybridization or amplification assays that include, but are not limited to, Southern or Northern analyses, polymerase chain reaction analyses, and probe arrays. One method for the detection of mRNA levels involves contacting the isolated mRNA with a nucleic acid polymer (probe) that hybridizes to the mRNA encoded by the gene being detected. The nucleic acid probe comprises of, for example, a full-length cDNA, or a portion thereof, such as an oligonucleotide of at least 7, 15, 30, 50, 100, 250, or 500 nucleotides in length and sufficient to specifically hybridize under stringent conditions to an mRNA or genomic DNA encoding a biomarker, biomarker described herein above. Hybridization of an mRNA with the probe indicates that the biomarker or other target protein of interest is being expressed.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in a gene chip array. A skilled artisan readily adapts known mRNA detection methods for use in detecting the level of mRNA encoding the biomarkers or other proteins of interest.

An alternative method for determining the level of an mRNA of interest in a sample involves the process of nucleic acid amplification, e.g., by RT-PCR (see, for example, U.S. Pat. No. 4,683,202), ligase chain reaction (Barany (1991) Proc. Natl. Acad. Sci. USA 88:189 193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (U.S. Pat. No. 5,854,033), or any other nucleic acid amplification method, followed by the detection of the amplified molecules using techniques well known to those of skill in the art. These detection schemes are especially useful for the detection of nucleic acid polymers if such molecules are present in very low numbers. In particular aspects of the invention, biomarker expression is assessed by quantitative fluorogenic RT-PCR (i.e., the TaqMan System).

Expression levels of an RNA of interest are monitored using a membrane blot (such as used in hybridization analysis such as Northern, dot, and the like), or microwells, sample tubes, gels, beads, or fibers (or any solid support comprising bound nucleic acids). See U.S. Pat. Nos. 5,770,722, 5,874,219, 5,744,305, 5,677,195 and 5,445,934, which are incorporated herein by reference. The detection of expression also comprises using nucleic acid probes in solution.

In some embodiments, microarrays are used to determine expression of one or more biomarkers. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See U.S. Pat. Nos. 6,040,138, 5,800,992, 6,020,135, 6,033,860, 6,344,316, and U.S. Pat. Application 20120208706. High-density oligonucleotide arrays are particularly useful for determining the gene expression profile for a large number of RNAs in a sample. Exemplary microarray chips include FoundationOne and FoundationOne Heme from Foundation Medicine, Inc; GeneChip® Human Genome U133 Plus 2.0 array from Affymetrix; and Human DiscoveryMAP® 250+v. 2.0 from Myraid RBM.

Techniques for the synthesis of these arrays using mechanical synthesis methods are described in, e.g., U.S. Pat. No. 5,384,261. In some embodiments, an array is fabricated on a surface of virtually any shape or even a multiplicity of surfaces. In some embodiments, an array is a planar array surface. In some embodiments, arrays include peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, each of which is hereby incorporated in its entirety for all purposes. In some embodiments, arrays are packaged in such a manner as to allow for diagnostics or other manipulation of an all-inclusive device.

Any means for specifically quantifying a biomarker in the biological sample of a candidate subject is contemplated. Thus, in some embodiments, expression level of a biomarker protein of interest in a biological sample is detected by means of a binding protein capable of interacting specifically with that biomarker protein or a biologically active variant thereof. In some embodiments, labeled antibodies, binding portions thereof, or other binding partners are used. The word “label” when used herein refers to a detectable compound or composition that is conjugated directly or indirectly to the antibody so as to generate a “labeled” antibody. In some embodiments, the label is detectable by itself (e.g., radioisotope labels or fluorescent labels) or, in the case of an enzymatic label, catalyzes chemical alteration of a substrate compound or composition that is detectable.

The antibodies for detection of a biomarker protein are either monoclonal or polyclonal in origin, or are synthetically or recombinantly produced. The amount of complexed protein, for example, the amount of biomarker protein associated with the binding protein, for example, an antibody that specifically binds to the biomarker protein, is determined using standard protein detection methodologies known to those of skill in the art. A detailed review of immunological assay design, theory and protocols are found in numerous texts in the art (see, for example, Ausubel et al., eds. (1995) Current Protocols in Molecular Biology) (Greene Publishing and Wiley-Interscience, NY)); Coligan et al., eds. (1994) Current Protocols in Immunology (John Wiley & Sons, Inc., New York, N.Y.).

The choice of marker used to label the antibodies will vary depending upon the application. However, the choice of the marker is readily determinable to one skilled in the art. These labeled antibodies are used in immunoassays as well as in histological applications to detect the presence of any biomarker of interest. The labeled antibodies are either polyclonal or monoclonal. Further, the antibodies for use in detecting a protein of interest are labeled with a radioactive atom, an enzyme, a chromophoric or fluorescent moiety, or a colorimetric tag as described elsewhere herein. The choice of tagging label also will depend on the detection limitations desired. Enzyme assays (ELISAs) typically allow detection of a colored product formed by interaction of the enzyme-tagged complex with an enzyme substrate. Radionuclides that serve as detectable labels include, for example, 1-131, 1-123, 1-125, Y-90, Re-188, Re-186, At-211, Cu-67, Bi-212, and Pd-109. Examples of enzymes that serve as detectable labels include, but are not limited to, horseradish peroxidase, alkaline phosphatase, beta-galactosidase, and glucose-6-phosphate dehydrogenase. Chromophoric moieties include, but are not limited to, fluorescein and rhodamine. The antibodies are conjugated to these labels by methods known in the art. For example, enzymes and chromophoric molecules are conjugated to the antibodies by means of coupling agents, such as dialdehydes, carbodiimides, dimaleimides, and the like. Alternatively, conjugation occurs through a ligand-receptor pair. Examples of suitable ligand-receptor pairs are biotin-avidin or biotin-streptavidin, and antibody-antigen.

In certain embodiments, expression of one or more biomarkers of interest within a biological sample, for example, a cell sample, is determined by radioimmunoassays or enzyme-linked immunoassays (ELISAs), competitive binding enzyme-linked immunoassays, dot blot (see, for example, Promega Protocols and Applications Guide, Promega Corporation (1991), Western blot (see, for example, Sambrook et al. (1989) Molecular Cloning, A Laboratory Manual, Vol. 3, Chapter 18 (Cold Spring Harbor Laboratory Press, Plainview, N.Y.), chromatography such as high performance liquid chromatography (HPLC), or other assays known in the art. Thus, the detection assays involve steps such as, but not limited to, immunoblotting, immunodiffusion, immunoelectrophoresis, or immunoprecipitation.

In some embodiments, the activities of the Trk receptor are measured by methods such as co-immunoprecipitation, fluorescence spectroscopy, fluorescence resonance energy transfer (FRET), isothermal titration calorimetry (ITC), dynamic light scattering (DLS), surface plasmon resonance (SPR), or other similar methods.

In some embodiments, Trk receptors are measured using in vivo imaging techniques, such as PET, OCT, MRI; combined with labeled or unlabeled antibodies, peptides, small molecules or related binding agents of Trk receptor.

Samples

In certain embodiments, one or more of the methods disclosed herein comprise a sample. In some embodiments, the sample is a cell sample or a tissue sample. In some instances, the sample is a cell sample. In additional instances, the sample is a tissue sample. In some embodiments, the sample for use with the methods described herein is obtained from cells or tissues of an animal. In some instances, the animal is a human, a non-human primate, or a rodent.

In some embodiments, the cell or tissue sample comprises neurons or glial cells (or neuroglia). In some instances, neurons comprise sensory neurons, interneurons, or motor neurons. In some embodiments, glial cells are non-neuronal cells that maintain homeostasis, form myelin, and provide support and protection for neurons in the central and peripheral nervous systems. In some embodiments, glial cells further comprise astrocytes, microglia, and Müller glia. Astrocytes or astroglia are star-shaped glial cells located in the brain and spinal cord. Microglia are macrophages that comprises phagocytosis function and protect neurons of the central nervous system. Müller glia are a type of retinal glial cells that maintain the stability of the retinal extracellular environment by regulation of K+ levels, uptake of neurotransmitters, removal of debris, storage of glycogen, electrical insulation of receptors and other neurons, and mechanical support of the neural retina. In some embodiments, the cell or tissue sample comprises astrocytes. In some embodiments, the cell or tissue sample comprises microglia. In some embodiments, the cell or tissue sample comprises Müller glia. In some embodiments, the cell or tissue sample comprises neurons such as sensory neurons, interneurons, or motor neurons.

### Kits/Article of Manufacture

Disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include one or more of the pharmaceutical compositions described herein comprising an agonist of a Trk receptor and an excipient and/or delivery vehicle. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers, or other characters forming the label are attached, molded, or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

The terms “effective amount” or “therapeutically effective amount,” as used herein, refer to a sufficient amount of the non-natural Trk receptor agonist being administered that would be expected to relieve to some extent one or more of the symptoms of the disease or condition being treated. For example, an “effective amount” for therapeutic uses is the amount of non-natural Trk receptor agonist, including a formulation as disclosed herein required to provide a decrease or amelioration in disease symptoms without undue adverse side effects. The term “therapeutically effective amount” includes, for example, a prophylactically effective amount. An “effective amount” disclosed herein is an amount effective to achieve a desired pharmacologic effect or therapeutic improvement without undue adverse side effects. It is understood that “an effective amount” or “a therapeutically effective amount” varies, in some embodiments, from subject to subject, due to variation in metabolism of the compound administered, age, weight, general condition of the subject, the condition being treated, the severity of the condition being treated, and the judgment of the prescribing physician. It is also understood that “an effective amount” in an extended-release dosing format may differ from “an effective amount” in an immediate release dosing format based upon pharmacokinetic and pharmacodynamic considerations.

The terms “enhance” or “enhancing” refers to an increase or prolongation of either the potency or duration of a desired effect of non-natural Trk receptor agonist, or a diminution of any adverse symptomatology that is consequent upon the administration of the therapeutic agent. Thus, in regard to enhancing the effect of the non-natural Trk receptor agonists disclosed herein, the term “enhancing” refers to the ability to increase or prolong, either in potency or duration, the effect of other therapeutic agents that are used in combination with the non-natural Trk receptor agonist disclosed herein. An “enhancing-effective amount,” as used herein, refers to an amount of non-natural Trk receptor agonist or other therapeutic agent which is adequate to enhance the effect of another therapeutic agent or non-natural Trk receptor agonist in a desired system. When used in a patient, amounts effective for this use will depend on the severity and course of the disease, disorder or condition, previous therapy, the patient's health status and response to the drugs, and the judgment of the treating physician.

The term “inhibiting” includes preventing, slowing, or reversing the development of a condition, for example, or advancement of a condition in a patient necessitating treatment.

“Pharmacodynamics” refers to the factors which determine the biologic response observed relative to the concentration of drug at the desired site of interest.

“Pharmacokinetics” refers to the factors which determine the attainment and maintenance of the appropriate concentration of drug at the desired site of interest.

The term “non-natural Trk receptor agonist” includes agents that recognize and bind to one or more epitopes on a Trk receptor. For example, Trk receptors include TrkA, TrkB, or TrkC receptor. In some embodiments, the non-natural Trk receptor agonist is a neurotrophic agent. The non-natural Trk receptor agonists are agents that can promote the growth and/or regeneration of neurons and their processes and connections. In some embodiments, a non-natural Trk receptor agonist provides therapeutic benefit by promoting the growth and/or regeneration and/or phenotypic maintenance of neurons. In some embodiments, a non-natural Trk receptor agonist provides therapeutic benefit by treating and/or reversing damage to neurons or reducing or delaying further damage to neurons.

Non-natural Trk receptor agonists include “non-natural neurotrophic agent” which means a chemically modified analog of a naturally occurring neurotrophic agent (e.g., NGF, BDNF, NT-3, NT-4, NT-5), or a naturally occurring neurotrophic agent with one or more mutations in amino acid residues. In some embodiments, a non-natural neurotrophic agent is a growth factor. In some instances, the non-natural Trk receptor agonist is a modified neurotrophic agent (e.g., NGF, BDNF, NT-3, NT-4, NT-5) comprising one or more mutations in amino acid residues.

The term “mutein” refers to any protein arising as a result of a modification. In some instances, the modification is a mutation. In some embodiments, the “mutein” is a protein with altered amino acid sequences as compared to the wild-type protein from which the mutated protein is derived.

A “prodrug” refers to a non-natural Trk receptor agonist that is converted into the parent drug in vivo. In certain embodiments, a prodrug is enzymatically metabolized by one or more steps or processes to the biologically, pharmaceutically or therapeutically active form of the compound. To produce a prodrug, a pharmaceutically active compound is modified such that the active compound will be regenerated upon in vivo administration. In one embodiment, the prodrug is designed to alter the metabolic stability or the transport characteristics of a drug, to mask side effects or toxicity, or to alter other characteristics or properties of a drug. Compounds provided herein, in some embodiments, are derivatized into suitable prodrugs.

The terms “treat,” “treating” or “treatment,” as used herein, include alleviating, abating or ameliorating a disease or condition, for example tinnitus, symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### EXAMPLES

These examples are provided for illustrative purposes only and not to limit the scope of the claims provided herein.

### Example 1. Nerve Growth Factor Muteins

Wild-type NGF, mutein Δ9-13 (NGF-A), mutein 7-84-103 (NGF-B), and mutein KKE (NGF-C) were produced in baculovirus expression systems and purified. Muteins were purified by cation exchange chromatography followed by immunoaffinity on an anti-NGF mouse monoclonal antibody.

Each intraocular injection for each of these compounds consisted of a total amount of 1 μg in 2 μl volumes. The solvent was 20 mM Potassium Phosphate buffer (pH 6.7).

### Example 2. Chronic Retinal Ganglion Cell Death Induced by High Intraocular Pressure

Animals. Wistar rats (female, 250-300 g; Charles River) were kept in a 12 hours dark-light cycle with food and water ad libitum. All animal manipulations were performed between 9 AM and 12 AM.

Anesthesia. For deep anesthesia, a cocktail of ketamine, xylazine, and acepromazine was injected intraperitoneally at a ratio of 50:5:1, respectively mg/kg dose. Deep anesthesia was used for cauterization (glaucoma), optic nerve axotomy, fluorogold labeling, intraocular injection procedures, and euthanasia. For light anesthesia, a gas mixture of oxygen and 2% isofluorane at a rate of 1 liter/minute was used. Light anesthesia was used for measuring IOP.

Glaucoma Model. Episcleral vein cauterization (EVC) was performed as described in Shi, Z., E. Birman, and H. U. Saragovi, Dev Neurobiol, 2007. 67(7): p. 884-94. Briefly, radial incisions were made in conjunctiva and three of the episcleral veins (two dorsal episcleral veins located near the superior rectus muscle and one temporal episcleral vein located near the lateral rectus muscle) were cauterized with a 30″ cautery tip. The contralateral control eyes had sham-surgery to only isolate the three veins but without cauterization. Planar ophthalmoscopy was used to examine retinal blood circulation. The rare cases in which the retinal vasculature showed signs of ischemia were excluded from the study.

Intraocular Pressure (IOP) Measurement. IOP was measured by Tonopen XL applanation tonometer immediately and every week after the EVC surgery until the endpoint of the experiment. The mean normal IOP of rats under light anesthesia was 10-14 mm Hg while in EVC glaucoma model elevated IOP can last for as long as 4 months. Four consecutive readings were obtained from each eye, each with a coefficient of variation <5%, of which the average number was taken as the IOP for the day.

Optic Nerve Transection (Axotomy). The procedure was modified as described by Berry et al (Berry, M., J. Carlile, and A. Hunter, Peripheral nerve explants grafted into the vitreous body of the eye promote the regeneration of retinal ganglion cell axons severed in the optic nerve. J Neurocytol, 1996. 25(2): p. 147-70). Briefly, after shaving the head, a 1.5-2.0 cm skin incision was made along the edge of the right orbit bone. Lachrymal glands and orbital fats were excised and extraocular muscles were separated to expose the optic nerve. An 18 G needle was used to lacerate the sheath longitudinally. The optic nerve parenchyma was then separated out and lifted, and the optic nerve was completely transected 0.5-1.0 mm posterior to the eye ball with the use of micro-tweezers. Normal blood circulation in the retina was ascertained.

Intraocular Injections. The superior part of the conjunctiva was incised radially. A 30 G needle was inserted at a 45° angle 2 mm behind cornea-scleral limbal until all the bevel of the needle was inserted into the vitreous body and avoid damaging the lens. After the injection, the needle was left in place for another minute to make sure all the compounds were dispersed into vitreous. The needle was slowly withdrawn and immediately a drop of surgical glue (Indermil; Loctite Ltd. Ireland) was administrated onto the injection site to seal the sty. The experimental eyes were injected with test or control agents while the normal contralateral eyes served as controls.

Drug regimen. For the glaucoma model (endpoint at day 42 after cauterization) intraocular injections were performed at days 14 and 21 after cauterization. For the axotomy model (endpoint at day 14 after optic nerve sectioning) intraocular injections were performed the day of optic nerve transection (while the rat was still under general anesthesia). All injections delivered 2 ml intraocularly. Compounds were injected at a total dose of 1.0 mg.

Fluorogold Retrograde Labeling. Retinal ganglion cells (RGCs) were retrogradely labeled with a 4% Fluorogold solution (Fluorochrome, Englewood, Colo.) applied bilaterally to the superior colliculous (SC). Briefly, after general anesthesia and shaving of the head, rats were mounted on stereotactic apparatus (Kopf Instruments, Tujunga, Calif.). After an incision on the skull skin, holes were drilled (1.6 mm long, 3.0 mm wide and 6 mm deep), at a position 1.3 mm lateral to the sagittal suture and 2.5 mm anterior to the lambda suture on each side. Fluorogold (3 μl) was injected into the SC at the depth of 6.0 mm below the skull. After injection of the dye, a piece of gelfoam soaked 4% Fluorogold was put into the hole. In the glaucoma model, retrograde labeling was performed at day 35 after ocular hypertension (7 days before endpoint), while in the axotomy model retrograde labeling was carried out 7 days before optic nerve transection (21 days before endpoint).

RGCs Quantification. At the endpoint of each experiment, both eyes were enucleated, the anterior part cut out, and the remaining part was fixed into 4% paraformaldehyde (PFA). After 30 minutes fixation, retinas were flat-mounted on a glass slide and dissected by four radial cuts to facilitate flattening of the retinas with vitreous side up. Images were taken using a Zeiss fluorescence microscope (Carl Zeiss Meditec, Jena, Germany), with 12 pictures/retina. For each quadrant there were 3 pictures at 20× magnification, at the distance of 1 mm, 2 mm, and 3 mm radial from the optic nerve. Microglia and macrophages which incorporated Fluorogold after phagocytosis of dying RGCs were excluded according to their morphology. For each rat, an experimental eye (OD) and a normal contralateral eye (OS) was used. The automated quantitative analysis was done with “Metamorph” software.

RGC Survival and Statistical Analysis. Standardization of RGCs survival in each rat was calculated as the ratio of the OD versus OS (RGCₑₓₚₑᵣᵢₘₑₙₜₐₗ/RGC_{contralateral control}). For each experimental group (untreated, or treated with PBS, wild-type NGF, and NGF muteins), the OD/OS ratios of each rat were averaged ±SEM (n=5-11 rats per group). Data analysis was performed using GraphPad Prism 5 software (GraphPad Software Inc., San Diego, Calif.). Comparison between the RGCs survival rate is using Student t-tests with Bonferroni corrections, and p≤0.05 was considered statistically significant.

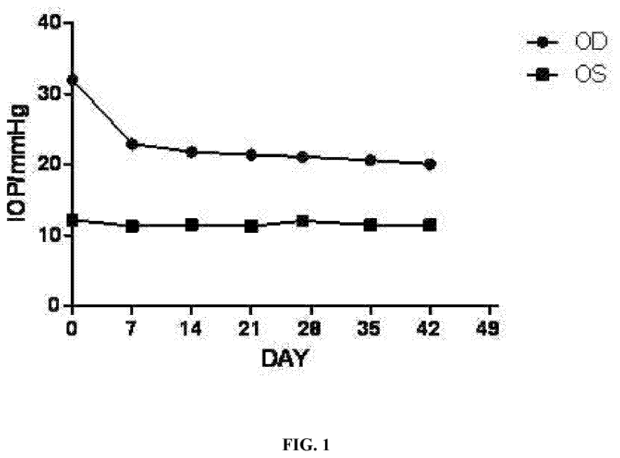

High IOP was induced in the right eye of rats by EVC (FIG. 1). The right eyes of each rat (OD, closed circles) were cauterized, and the left eyes of each rat (OS, closed squares) were normal IOP control. The IOP was measured on the indicated days (X-axis). The difference in IOP between the cauterized eye and the normal eye was significantly different at each of the times shown (p≤0.01).

### Example 3. TrkA Receptor Agonist Treatment of Glaucoma

RGC protection following TrkA agonist treatment was tested.

Wild-type NGF, mutein 49-13 (NGF-A), mutein 7-84-103 (NGF-B), and mutein KKE (NGF-C) described in Example 1 were used, and similar protocols described in Example 2 were used. Cauterization was performed to induce high IOP for 14 days. At that point, wild-type NGF, mutein 49-13 (NGF-A), mutein 7-84-103 (NGF-B), and mutein KKE (NGF-C) were injected intraocularly. Wild type NGF, saline vehicle, or no treatment were used as controls. Two independent intraocular injections each of 1 μg compound at days 14 and 21 after cauterization were performed. Surviving RGCs were identified by retrograde labeling and counted at day 42 post-surgery.

The data showing RGC survival in glaucoma (and axotomy) are presented in Table 3. The number of RGCs was counted in the OD and OS eyes of each rat after retrograde labeling of retinas as described in Example 2. Percent survival was calculated with respect to the normal contralateral control eye in each rat (pegged at 100%). The glaucomatous eyes (OD) of rats were treated as indicated at day 14 and day 21 of high IOP, and the normal contralateral eyes (OS) were intact and used as controls (100% survival). The axotomized eyes (OD) of rats were treated as indicated at day 1, and the normal contralateral eyes (OS) were intact and used as controls (100% survival). The receptor specificity of each mutant is indicated as + (active at that receptor) or − (inactive at that receptor).

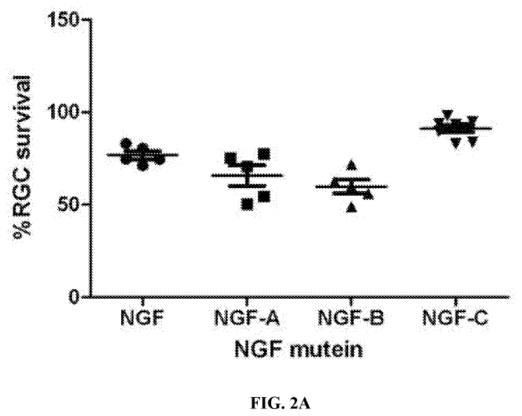

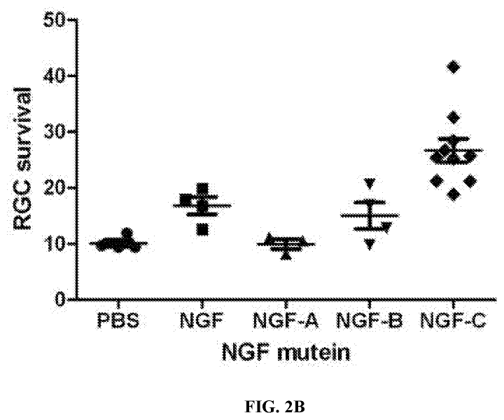

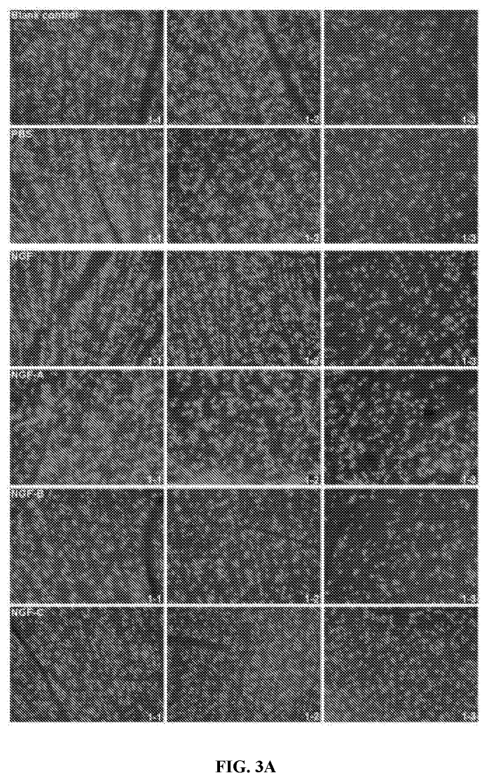

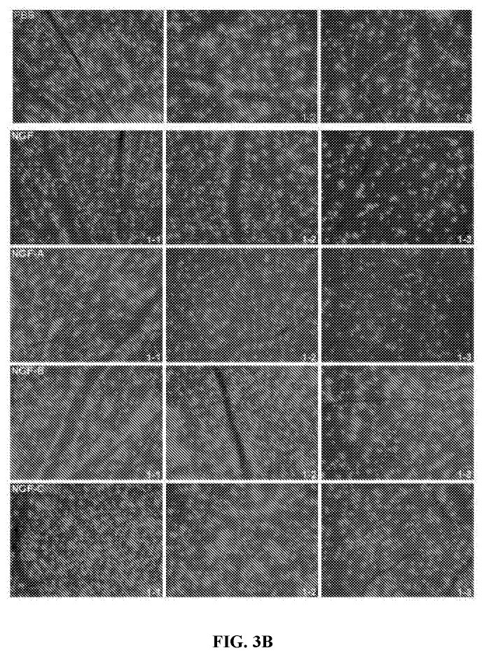

A graphic representation of these data is shown in FIG. 2A. Representative pictures of flat mounted retinas prepared to count RGCs are shown in FIG. 3A.

TABLE 3

| | | | | SURVIVAL RATE | |
|---|---|---|---|---|---|
| | | | | (MEAN ± SEM) | |
| | | RECEPTOR | | % OF INTACT | |
| | NGF | BINDING | | CONTRALATERAL | |
| | MUTEIN | SPECIFICITY | | EYE | |
| TREATMENT | NAME | TrkA | p75 | Glaucoma | Axotomy |
| Untreated | — | — | — | 74.0 ± 1.2 | 10 ± 0.4 |
| PBS control | — | — | — | 76.0 ± 2.2 | N/A |
| NGF-WT | wild type NGF | + | + | 77.7 ± 1.7 | 17 ± 1.1 |
| NGF-A | Δ 9/13 | — | + | 65.7 ± 3.7 | 9.9 ± 0.6 |
| NGF-B | 7-84-103 | +/− | + | 59.8 ± 2.5 | 15 ± 1.5 |
| NGF-C | KKE | + | — | 92.4 ± 1.7 | 28.4 ± 3.0 |

### Example 4: TrkA Receptor Agonist Treatment of Axotomy

RGC protection against apoptosis was tested.

Wild-type NGF, mutein 49-13 (NGF-A), mutein 7-84-103 (NGF-B), and mutein KKE (NGF-C) described in Example 1 were used, and similar protocols described in Example 2 were used. Complete optic nerve axotomy was performed to induce RGC loss. Wild-type NGF, mutein 49-13 (NGF-A), mutein 7-84-103 (NGF-B), and mutein KKE (NGF-C) were injected intraocularly. Wild type NGF, saline vehicle, and no treatment were used as controls. One intraocular injection each of 1 μg compound at day 1 post-axotomy was performed. Surviving RGCs, identified by retrograde labeling, were counted at day 14 post-axotomy. In this protocol no treatment of any type was performed between days 1 and 14, to evaluate whether any protection could be long-lived.

The data showing RGC survival in axotomy (and glaucoma) are presented in Table 3. A graphic representation of these data is shown in FIG. 2B. Representative pictures of flat mounted retinas prepared to count RGCs are shown in FIG. 3B. The data shows that selective TrkA receptor agonist NGF-C rescues RGCs in axotomy, and prevents any further RGC death from the time of initial treatment until the endpoint 14 days of continuous injury. In contrast, the p75 receptor agonists NGF-A and NGF-B each can accelerate RGCs death in glaucoma. Wild type NGF did not alter the rate of RGCs death in axotomy.

### Example 5: Nerve Growth Factor in Alzheimer's Model of Memory and Learning

Wild-type nerve growth factor (NGF) was tested in an Alzheimer's model for memory and learning.

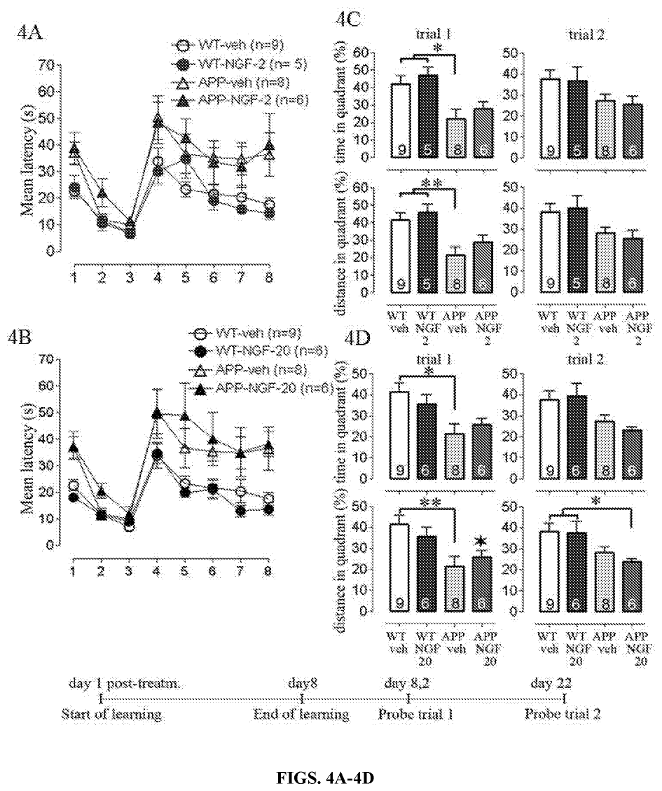

Wild-type NG) as described in Example 1 was used. Transgenic mice were generated that overexpress mutant amyloid-precursor protein (APP mice). Wild-type mice (WT) and APP mice were treated with either vehicle, 2 μg of NGF (NGF-2), or 20 μg of NGF (NGF-20). Referring to FIGS. 4A-4B, mean latency in seconds (Y-axis) was measured over 8 days (X-axis). As seen in FIG. 4A, NGF at 2 μg total dose (NGF-2) had no effect on the learning of APP mice. Mean latency in APP-NGF-2 group was comparable to that in the APP-vehicle group, and both APP groups had higher latency compared to wild-type mice groups. As seen in FIG. 4B, treatment with 20 μg total dose of NGF (NGF-20) did not improve learning capacity of APP mice. Mean latency was similar in both APP groups, and was higher than wild-type groups (day 5 and day 8,* p<0.05 APP-NGF-20 vs. wild-type groups; day 8, * p<0.05 APP-veh. vs. wild-type groups)

Short-term memory and long-term memory were also measured. Short-term memory (trial 1) and long-term memory (trial 2) were measured in mice treated with 2 μg of NGF (FIG. 4C) and mice treated with 20 μg of NGF (FIG. 4D). Trial 1 was performed hours after completion of Morris Water Maze (MWM) and Trial 2 was performed 2 weeks after completion of the MWM. Referring to FIGS. 4C-4D, NGF-2 and NGF-20 had no effect on memory deficits in APP mice, and had no effects in wild-type mice (* p<0.05; **p<0.01). The number of animals used in probe trials for each groups are indicated in each histogram. Swim speeds were comparable between groups (data not shown). Error bars represent SEM.

The data shows that treatment with NGF (2 μg and 20 μg doses) did not improve the spatial learning deficits observed in APP mice, and had no effect in wild type mice (FIG. 4A). A higher dose of NGF (20 μg) did not improve the learning performance of APP mice, and had no effect in wild-type mice (FIG. 4B). Moreover, the performance of APP and wild-type mice in trials 1 and 2 were not affected by either doses of NGF (FIGS. 4A-4B). Thus, infusion of NGF had no effect on spatial learning or on memory, irrespective of whether the mice are cognitively impaired or not.

### Example 6: TrkA Selective Agonist NGF Mutein (NGF-C) Improves Learning in an Alzheimer's Model of Memory and Learning

TrkA receptor agonist mutein NGF-C was tested in an Alzheimer's mouse model for memory and learning.

Wild-type NGF and mutein KKE (NGF-C) as described in Example 1 were used.

Transgenic mice were generated that overexpress mutant amyloid-precursor protein (APP mice). Wild-type mice (WT) and APP mice were treated with vehicle or 5 μg of NGF-C.

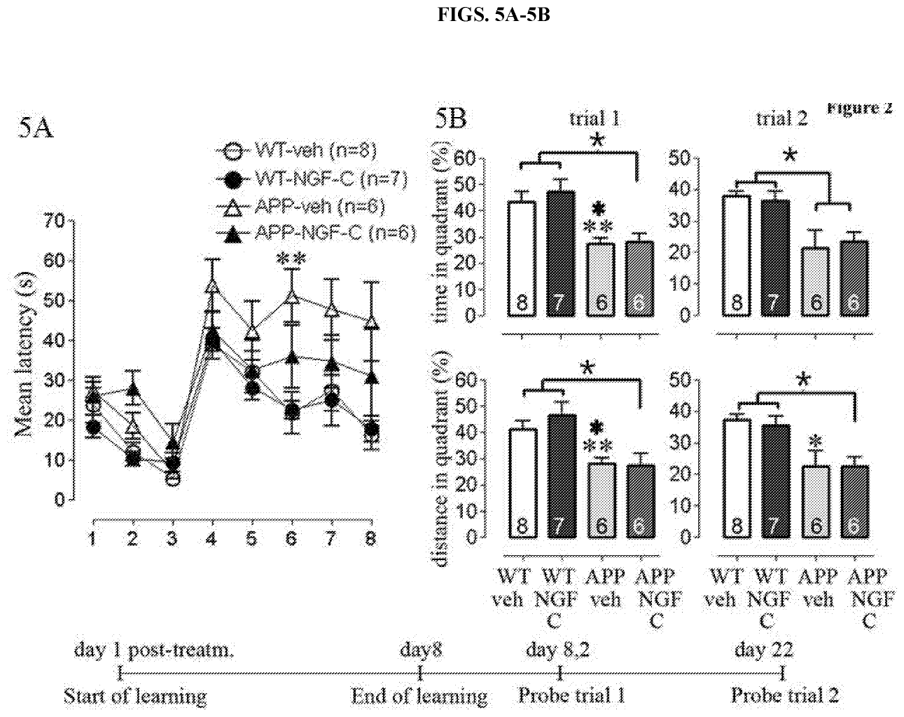

Referring to FIG. 5A, mean latency in seconds (Y-axis) was measured over 8 days (X-axis). A trend towards better learning was observed in APP mice treated with NGF-C. Because of the elevated SEM in all days of training, the mean latency was not significantly different between NGF-C-treated and vehicle-treated APP mice. On day 6, the difference between APP-vehicle and wild-type groups was significant (**p<0.01).

Effects of NGF-C on memory in wild-type and APP mice was also measured. Short-term memory (trial 1) and long-term memory (trial 2) were measured in mice treated with 5 μg of NGF-C(FIG. 5B). Trial 1 was performed hours after completion of Morris Water Maze (MWM) and Trial 2 was performed 2 weeks after completion of the MWM. Referring to FIG. 5B, NGF-C had no effect on memory in APP mice or wild-type mice. In trial 1, time and distance spent in the target quadrant were significantly different between wild-type groups and APP groups (*p<0.05; **p<0.01 APP-veh. vs wild-type-NGF-C; *p<0.05 APP-veh. vs wild-type-veh.). No long-term effect in wild-type mice was observed with NGF-C treatment (*p<0.05 APP-veh. vs. wild-type groups; *p<0.05). The number of animals used in probe trials for each groups are indicated in histogram. Swim speeds were comparable between groups (data not shown). Error bars represent SEM.

The data shows that the mutein NGF-C treatment of APP mice improved performance in Morris Water Maze learning and short term memory compared to vehicle-treated APP mice (FIG. 5A). NGF-C-treated APP mice showed no improvement in trials testing for long term memory (FIG. 5B). Similar treatment of wild-type mice with NGF-C showed no changes in learning or in memory (FIGS. 5A-5B). These behavioral data suggest that selective targeting of TrkA have some benefits in disease states.

### Example 7—Interaction of TrkC with Exemplary NT-3 Mutants

In this study, exemplary NT-3 mutants (SEQ ID NOs: 23, 31, and 32) were profiled for their respective interaction with TrkC and p75 in the presence of wild type NT-3. NT-3 mutants (SEQ ID NOs: 23 and 32) were observed to have reduced binding to p75 relative to wild type NT-3.

Wild type NT-3 was covalently linked to biotin for use as a tracer. In this study, the presence of the covalently linked biotin was identified using a fluorescent-avidin in fluocytometry studies with mean channel fluorescence (MCF) indicating the level of binding. Assays were based on Barcelona, et al., Mol Cell Biol. 35(19):3396-3408 (2015).

Unlabeled wild type NT3 or exemplary mutant NT3 proteins were used as competitors (10-fold excess) as “cold competitors” to competitively block binding of Biotin-NT-3 to receptors. In the absence of competitor, Biotin-NT-3 bound to cells expressing the indicated receptor TrkC or p75 (Table 4 lane 2) and was standardized to 100% binding. Unlabeled wild type NT-3 competitively blocked binding of Biotin-NT-3 to TrkC or to p75 receptors to levels of about 18% and 17% of maximal levels (Table 4 lane 3). An exemplary NT-3 mutant (SEQ ID NO: 31) competed with Biotin-NT-3 for binding to TrkC and to p75 to levels of about 8% and 15% of maximal levels, respectively (Table 4 lane 4). A second NT-3 mutant (SEQ ID NO: 32) competed with Biotin-NT-3 for binding to TrkC to a level of about 2%, and with Biotin-NT-3 for binding to p75 to a level of about 78% of maximal levels (Table 4 lane 5). A third NT-3 mutant (SEQ ID NO: 23) competed with Biotin-NT-3 for binding to TrkC to a level of about 13%, and with Biotin-NT-3 for binding to p75 to a level of about 89% of maximal levels (Table 4 lane 6).

TABLE 4

| | | | | % of | | % of |
|---|---|---|---|---|---|---|
| | Biotin- | | NIH- | maximal | NIH- | maximal |
| | NT3 | Test or | TrkC | binding | p75 | binding |
| Lane | (nM) | cold competitor | MCF | to TrkC | MCF | to p75 |
| 1 | 0 | Background | 10 | 0 | 11 | 0 |
| | | (no biotin-NT3) | | | | |
| 2 | 20 | No competition | 93 | 100 | 76 | 100 |
| 3 | 20 | Wild type NT3 | 25 | 18 | 22 | 17 |
| | | 200 nM | | | | |
| 4 | 20 | NT-3 mutant (SEQ ID | 17 | 8 | 21 | 15 |
| | | NO: 31) at 200 nM | | | | |
| 5 | 20 | NT-3 mutant (SEQ ID | 12 | 2 | 62 | 78 |
| | | NO: 32) at 200 nM | | | | |
| 6 | 20 | NT-3 mutant (SEQ ID | 21 | 13 | 69 | 89 |
| | | NO: 23) at 200 nM | | | | |

### Example 8—Exemplary NT-3 Mutants Activate TrkC and Promote Survival Signals

Wild type NT-3 or NT-3 mutant proteins (SEQ ID NOs: 23, 31, 32) were tested as survival factors for cells that express TrkC. Cells were cultured in serum free media to cause stress and death. Supplementation with a growth factor (NT3 or NT3 mutants for the TrkC-expressing cells) prevented death caused by culture in serum free media. MTT assay as described in Brahimi et al, PLoS One 11(10): e0162307 (2016) was used and the colorimetrical readout was in Optical Density unit.

Without supplementation, TrkC-expressing cells did not survive and did not metabolize MTT (Table 5 lane 1). Addition of supplementation to wild type NT3 promoted cell survival in a dose dependent manner. 1 nM wild type NT3 was standardized as 100% (Table 5 lanes 2 and 3). Addition of supplementation to NT3 mutants (SEQ ID NOs: 23, 31, and 32) promoted cell survival in a dose dependent manner (Table 5 lanes 4,5 and 6,7 and 8,9).

TABLE 5

| | | Concentration | MTT | % |
|---|---|---|---|---|
| Lane | Test Growth Factor | (nM) in culture | O.D. | survival |
| 1 | No treatment (background) | 0 | 0.04 | 0 |
| 2 | Wild type NT3 | 0.1 | 0.30 | 34 |
| 3 | | 1 | 0.81 | 100 |
| 4 | NT-3 mutant (SEQ ID NO: 31) | 0.1 | 0.28 | 31 |
| 5 | | 1 | 0.87 | 108 |
| 6 | NT-3 mutant (SEQ ID NO: 32) | 0.1 | 0.34 | 39 |
| 7 | | 1 | 0.92 | 114 |
| 8 | NT-3 mutant (SEQ ID NO: 23) | 0.1 | 0.27 | 30 |
| 9 | | 1 | 0.89 | 110 |

### Example 9—Interaction of TrkA with Exemplary NT-3 Mutants

In this study, exemplary NT-3 mutants (SEQ ID NOs: 23, 31, and 32) were profiled for their respective interaction with TrkA and were observed to have higher binding interaction with TrkA relative to wild type NT-3.

Wild type NT-3 was covalently linked to biotin for use as a tracer. In this study, the presence of the covalently linked biotin was identified using a fluorescent-avidin in fluocytometry studies with mean channel fluorescence (MCF) indicating the level of binding. Assays were based on Barcelona, et al., Mol Cell Biol. 35(19):3396-3408 (2015).

Unlabeled wild type NT3 or exemplary mutant NT3 proteins were used as competitors (3-fold excess) as “cold competitors” to competitively block binding of Biotin-NT-3 to receptors.

In the absence of competitor, Biotin-NT-3 bound to cells expressing TrkA (Table 6 lane 2) and was standardized to 100% binding. Unlabeled wild type NT-3 competitively blocked binding of Biotin-NT-3 to TrkA receptors and binding was reduced to about 36% of maximal levels (Table 6 lane 3). An exemplary NT-3 mutant (SEQ ID NO: 31) exhibited reduced binding to TrkA in the presence of Biotin-NT-3 and binding remained at 79% of maximal levels (Table 6 lane 4). A second exemplary NT-3 mutant (SEQ ID NO: 32) was observed to compete with Biotin-NT-3 for binding to TrkA and binding was reduced to 46% of maximal levels (Table 6 lane 5). A third exemplary NT-3 mutant (SEQ ID NO: 23) competed with Biotin-NT-3 for binding to TrkA with higher efficacy than wild type NT-3, and binding was reduced to 21% of maximal levels (Table 6 lane 6).

TABLE 6

| | | | | % of |
|---|---|---|---|---|
| | | | | maximal |
| | Biotin-NT3 | Test or | NIH-TrkA | binding |
| Lane | (nM) | cold competitor | MCF | to TrkA |
| 1 | 0 | Background (no biotin-NT3) | 10 | 0 |
| 2 | 20 | No competition | 38 | 100 |
| 3 | 20 | Wild type NT3 | 20 | 36 |
| 4 | 20 | NT-3 mutant (SEQ ID NO: 31) | 32 | 79 |
| 5 | 20 | NT-3 mutant (SEQ ID NO: 32) | 23 | 46 |
| 6 | 20 | NT-3 mutant (SEQ ID NO: 23) | 16 | 21 |

The examples and embodiments described herein are for illustrative purposes only and various modifications or changes suggested to persons skilled in the art are to be included within the spirit and purview of this application and scope of the appended claims.

1. A neurotrophic agent comprising modifications at amino acid residue positions equivalent to amino acid residues 15, 114, and 115 or to amino acid residues 15 and 103 set forth in SEQ ID NO: 2, wherein the neurotrophic agent is selected from NGF, NT-4, NT-5, BDNF or PNT.

2. The neurotrophic agent of claim 1, wherein the neurotrophic agent further comprises a modification at an amino residue position equivalent to at least one of amino acid residue 11, 68, 87, and 103 set forth in SEQ ID NO: 2.

3. A neurotrophic agent comprising modifications at amino acid residue positions equivalent to amino acid residues 7 and 103 set forth in SEQ ID NO: 1.

4. The neurotrophic agent of claim 3, wherein the neurotrophic agent further comprises:
a) a modification at an amino acid residue position equivalent to amino acid residue 84 set forth in SEQ ID NO: 1;
b) a modification at an amino acid residue position equivalent to amino acid residue 45 set forth in SEQ ID NO: 1; or
c) modifications at amino acid residue positions equivalent to amino acid residues 32, 34, and 35 set forth in SEQ ID NO: 1.

5. The neurotrophic agent of claim 3, wherein the neurotrophic agent comprises nerve growth factor (NGF), a pro-form of nerve growth factor (proNGF), neurotrophin-3 (NT-3), a pro-form of neurotrophin-3 (proNT-3), neurotrophin-4 (NT-4), a pro-form of neurotrophin-4 (proNT-4), neurotrophin-5 (NT-5), a pro-form of neurotrophin-5 (proNT-5), brain-derived neurotrophic factor (BDNF), a pro-form of brain-derived neurotrophic factor (proBDNF), a pan-neurotrophin (PNT), or PNT-1.

6. The neurotrophic agent of claim 3, wherein the neurotrophic agent is NGF, and wherein NGF comprises:
a) modifications at amino acid residue positions equivalent to amino acid residue 7, 84, and 103 set forth in SEQ ID NO: 1;
b) modifications at amino acid residue positions equivalent to amino acid residue 7, 45, and 103 set forth in SEQ ID NO: 1; or
c) about 70%, 80%, 85%, 90%, 91%, 92%, 93%, 94%, 95%, 96%, 97%, 98% or 99% sequence identity to SEQ ID NO: 8, 9, 10 or 11.

7. The neurotrophic agent of claim 6, wherein NGF further comprises modifications at amino acid residue positions equivalent to amino acid residues 32, 34, and 35 set forth in SEQ ID NO: 1.

8. A neurotrophic agent comprising at least two modifications selected from amino acid residue positions equivalent to amino acid residues (i) 74, 75, and 77, (ii) 115 and 116, (iii) 61 and 100, or (iv) 118 set forth in SEQ ID NO: 5.

9. The neurotrophic agent of claim 8, wherein the neurotrophic agent further comprises a modification at an amino acid residue position equivalent to amino acid residue 32, 34, 115, or 116 set forth in SEQ ID NO: 5.

10. The neurotrophic agent of claim 9, wherein the neurotrophic agent comprises nerve growth factor (NGF), neurotrophin-3 (NT-3), neurotrophin-4 (NT-4), neurotrophin-5 (NT-5), or brain-derived neurotrophic factor (BDNF).

11. The neurotrophic agent of claim 8, wherein the neurotrophic agent is a pan-neurotrophin (PNT), and wherein PNT comprises modifications at amino acid residue positions equivalent to amino acid residues 74, 75, and 77 set forth in SEQ ID NO: 5, and optionally comprises modifications at amino acid residue positions equivalent to amino acid residues 32, 34, 115, or 116 set forth in SEQ ID NO: 5.

12. The neurotrophic agent of claim 8, wherein PNT comprises about 70%, 80%, 85%, 90%, 91%, 92%, 93%, 94%, 95%, 96%, 97%, 98% or 99% sequence identity to SEQ ID NO: 14 or 16.

13. (canceled)

14. The neurotrophic agent of claim 8, wherein the neurotrophic agent is PNT, and wherein PNT comprises modifications at amino acid residue positions equivalent to amino acid residues 32 and 34 set forth in SEQ ID NO: 5, and optionally comprises modifications at amino acid residue positions equivalent to amino acid residues 74, 75, 77, 115, and 116 set forth in SEQ ID NO: 5.

15. The neurotrophic agent of claim 14, wherein the neurotrophic agent comprises about 70%, 80%, 85%, 90%, 91%, 92%, 93%, 94%, 95%, 96%, 97%, 98%, or 99% sequence identity to SEQ ID NO: 13 or 16.

16. (canceled)

17. The neurotrophic agent of claim 8, wherein the neurotrophic agent is PNT, and wherein PNT comprises modifications at amino acid residue positions equivalent to amino acid residues 115 and 116 set forth in SEQ ID NO: 5, and optionally comprises modifications at amino acid residue positions equivalent to amino acid residues 32, 34, 74, 75, and 77 set forth in SEQ ID NO: 5.

18. The neurotrophic agent of claim 17, wherein the neurotrophic agent comprises about 70%, 80%, 85%, 90%, 91%, 92%, 93%, 94%, 95%, 96%, 97%, 98%, or 99% sequence identity to SEQ ID NO: 15 or 16.

19. (canceled)

20. The neurotrophic agent of claim 8, wherein the neurotrophic agent is PNT, and wherein PNT comprises modifications at amino acid residue positions equivalent to amino acid residues 61 and 100 set forth in SEQ ID NO: 5.

21. The neurotrophic agent of claim 20, wherein the neurotrophic agent comprises about 70%, 80%, 85%, 90%, 91%, 92%, 93%, 94%, 95%, 96%, 97%, 98% or 99% sequence identity to SEQ ID NO: 20.

22. (canceled)

23. The neurotrophic agent of claim 8, wherein the neurotrophic agent is PNT, and wherein PNT comprises a modification at amino acid residue positions equivalent to amino acid residue 118 set forth in SEQ ID NO: 5.

24. The neurotrophic agent of claim 23, wherein the neurotrophic agent comprises about 70%, 80%, 85%, 90%, 91%, 92%, 93%, 94%, 95%, 96%, 97%, 98% or 99% sequence identity to SEQ ID NO: 21.

25.-45. (canceled)


SEQUENCE LISTING (34 sequences)

SEQ ID NO: 1 - protein, 120 aa, Unknown
  Description of Unknown Nerve Growth Factor sequence
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 2 - protein, 119 aa, Unknown [sequence omitted]

SEQ ID NO: 3 - protein, 130 aa, Unknown
  Description of Unknown Neurotrophin 4/5 sequence
GVSETAPASRRGELAVCDAVSGWVTDRRTAVDLRGREVEVLGEVPAAGGSPLRQYFFETRCKADNAEEGGPGAGGGGCRGVDRRHWVSECKAKQSYVRALTADAQGRVGWRWIRIDTACVCTLLSRTGRA

SEQ ID NO: 4 - protein, 119 aa, Unknown
  Description of Unknown Brain-derived neurotrophic factor sequence
HSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 5 - protein, 120 aa, Artificial Sequence
  Description of Artificial Sequence Synthetic polypeptide
SSSHPIFHRGEYSVCDSESLWVTDKSSAIDIRGHQVTVLGEIKTGNSPVKQYFYETRCKEARPVKNGCRGIDDKHWNSQCKTSQTYVRALTSESKKRIGWRWIRIDTSCVCALSRKIGRT

SEQ ID NO: 6 - protein, 121 aa, Artificial Sequence
  Description of Artificial Sequence Synthetic polypeptide
SSSHPIFHRGEFSVCDSVSVWVGDKTTATDIKGKEVMVLGEVNINNSVFKQYFFETKCNPMGYTKEGCRGIDSKHWNSQCRTTQSYVRALTMDSKKRIGWRFIRIDTACVCVLSRKAVRRA

SEQ ID NO: 7 - protein, 123 aa, Artificial Sequence
  Description of Artificial Sequence Synthetic polypeptide
SSSHPIFHRGEFSVCDSVSVWVTAADKKTAVDMSGGTVMVLGEVNINNSVFKQYFFETKCNPMGYTKEGCRGIDSKHWNSQCRTTQSYVRALTMDSKKRIGWRFIRIDTACVCVLSRKAVRRA

SEQ ID NO: 8 - protein, 120 aa, Artificial Sequence
  Description of Artificial Sequence Synthetic polypeptide
SSSHPIAHRGEFSVCDSVSVWVGDKTTATDIKGKEVMVLGEVNINNSVFKQYFFETKCRDPNPVDSGCRGIDSKHWNSYCTTTATFVKALTMDGKQAAWRFIAIDTACVCVLSRKAVRRA

SEQ ID NO: 9 - protein, 120 aa, Artificial Sequence
  Description of Artificial Sequence Synthetic polypeptide
SSSHPIAHRGEFSVCDSVSVWVGDKTTATDIKGKEVMVLGEVNIANSVFKQYFFETKCRDPNPVDSGCRGIDSKHWNSYCTTTHTFVKALTMDGKQAAWRFIAIDTACVCVLSRKAVRRA

SEQ ID NO: 10 - protein, 120 aa, Artificial Sequence
  Description of Artificial Sequence Synthetic polypeptide
SSSHPIAHRGEFSVCDSVSVWVGDKTTATDIAGAAVMVLGEVNINNSVFKQYFFETKCRDPNPVDSGCRGIDSKHWNSYCTTTATFVKALTMDGKQAAWRFIAIDTACVCVLSRKAVRRA

SEQ ID NO: 11 - protein, 120 aa, Artificial Sequence
  Description of Artificial Sequence Synthetic polypeptide
SSSHPIAHRGEFSVCDSVSVWVGDKTTATDIAGAAVMVLGEVNIANSVFKQYFFETKCRDPNPVDSGCRGIDSKHWNSYCTTTHTFVKALTMDGKQAAWRFIAIDTACVCVLSRKAVRRA

SEQ ID NO: 12 - protein, 115 aa, Artificial Sequence
  Description of Artificial Sequence Synthetic polypeptide
SSSHPIFHVCDSVSVWVGDKTTATDIAGAAVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 13 - protein, 120 aa, Artificial Sequence
  Description of Artificial Sequence Synthetic polypeptide
SSSHPIFHRGEYSVCDSESLWVTDKSSAIDIAGAQVTVLGEIKTGNSPVKQYFYETRCKEARPVKNGCRGIDDKHWNSQCKTSQTYVRALTSESKKRIGWRWIRIDTSCVCALSRKIGRT

SEQ ID NO: 14 - protein, 120 aa, Artificial Sequence
  Description of Artificial Sequence Synthetic polypeptide
SSSHPIFHRGEYSVCDSESLWVTDKSSAIDIRGHQVTVLGEIKTGNSPVKQYFYETRCKEARPVKNGCRGIDDAAWASQCKTSQTYVRALTSESKKRIGWRWIRIDTSCVCALSRKIGRT

SEQ ID NO: 15 - protein, 120 aa, Artificial Sequence
  Description of Artificial Sequence Synthetic polypeptide
SSSHPIFHRGEYSVCDSESLWVTDKSSAIDIRGHQVTVLGEIKTGNSPVKQYFYETRCKEARPVKNGCRGIDDKHWNSQCKTSQTYVRALTSESKKRIGWRWIRIDTSCVCALSAAIGRT

SEQ ID NO: 16 - protein, 120 aa, Artificial Sequence
  Description of Artificial Sequence Synthetic polypeptide
SSSHPIFHRGEYSVCDSESLWVTDKSSAIDIAGAQVTVLGEIKTGNSPVKQYFYETRCKEARPVKNGCRGIDDAAWASQCKTSQTYVRALTSESKKRIGWRWIRIDTSCVCALSAAIGRT

SEQ ID NO: 17 - protein, 121 aa, Artificial Sequence
  Description of Artificial Sequence Synthetic polypeptide
SSSHPIFHRGEFSVCDSVSVWVGDKTTATDIAGAAVMVLGEVNINNSVFKQYFFETKCNPMGYTKEGCRGIDSKHWNSQCRTTQSYVRALTMDSKKRIGWRFIRIDTACVCVLSRKAVRRA

SEQ ID NO: 18 - protein, 123 aa, Artificial Sequence
  Description of Artificial Sequence Synthetic polypeptide
SSSHPIFHRGEFSVCDSVSVWVTAADKKTAVDMAGAAVMVLGEVNINNSVFKQYFFETKCNPMGYTKEGCRGIDSKHWNSQCRTTQSYVRALTMDSKKRIGWRFIRIDTACVCVLSRKAVRRA

SEQ ID NO: 19 - protein, 120 aa, Artificial Sequence
  Description of Artificial Sequence Synthetic polypeptide
SSSHPIFHRGEYSVCDSESLWVTDKSSAIDIAGAAVTVLGEIKTGNSPVKQYFYETRCKEARPVKNGCRGIDDKHWNSQCKTSQTYVRALTSESKKRIGWRWIRIDTSCVCALSRKIGRT

SEQ ID NO: 20 - protein, 120 aa, Artificial Sequence
  Description of Artificial Sequence Synthetic polypeptide
SSSHPIFHRGEYSVCDSESLWVTDKSSAIDIRGHQVTVLGEIKTGNSPVKQYFYETRCKESRPVKNGCRGIDDKHWNSQCKTSQTYVRALTSESKKRIGWEWIRIDTSCVCALSRKIGRT

SEQ ID NO: 21 - protein, 120 aa, Artificial Sequence
  Description of Artificial Sequence Synthetic polypeptide
SSSHPIFHRGEYSVCDSESLWVTDKSSAIDIRGHQVTVLGEIKTGNSPVKQYFYETRCKEARPVKNGCRGIDDKHWNSQCKTSQTYVRALTSESKKRIGWRWIRIDTSCVCALSRKIART

SEQ ID NO: 22 - protein, 119 aa, Artificial Sequence
  Description of Artificial Sequence Synthetic polypeptide
YAEHKSHRGEYSVCASESLWVTDKSSAIDIRGHQVTVLGEIKTGNSPVKQYFYETRCKEARPVKNGCRGIDDKHWNSQCKTSQTYVRALTSENNKLVGWRWIAIDTSCVCALSRKIGRT

SEQ ID NO: 23 - protein, 119 aa, Artificial Sequence
  Description of Artificial Sequence Synthetic polypeptide
YAEHKSHRGEYSVCASESLWVTDKSSAIDIRGHQVTVLGEIKTGNSPVKQYFYETRCKEARPVKNGCRGIDDKHWNSQCKTSQTYVRALTSENNKLVGWRWIRIDTSCVCALSAAIGRT

SEQ ID NO: 24 - protein, 119 aa, Artificial Sequence
  Description of Artificial Sequence Synthetic polypeptide
YAEHKSHRGEASVCASESLWVTDKSSAIDIRGHQVTVLGEIKTGNSPVKQYFYETRCKEARPVKNGCAGIDDKHWNSQCKTSQTYVAALTSENNKLVGWRWIRIDTSCVCALSAAIGRT

SEQ ID NO: 25 - protein, 119 aa, Artificial Sequence
  Description of Artificial Sequence Synthetic polypeptide
YAEHKSHRGEYSVCASESLWVTDKSSAIDIRGHQVTVLGEIKTGNSPVKQYFYETRCKEARPVKNGCAGIDDKHWNSQCKTSQTYVAALTSENNKLVGWRWIRIDTSCVCALSAAIGRT

SEQ ID NO: 26 - protein, 119 aa, Artificial Sequence
  Description of Artificial Sequence Synthetic polypeptide
YAEHKSHRGEASVCASESLWVTDKSSAIDIRGHQVTVLGEIKTGNSPVKQYFYETRCKEARPVKNGCRGIDDKHWNSQCKTSQTYVAALTSENNKLVGWRWIRIDTSCVCALSAAIGRT

SEQ ID NO: 27 - protein, 119 aa, Artificial Sequence
  Description of Artificial Sequence Synthetic polypeptide
YAEHKSHRGEYSVCASESLWVTDKSSAIDIRGHQVTVLGEIKTGNSPVKQYFYETRCKEARPVKNGCRGIDDKHWNSQCKTSQTYVAALTSENNKLVGWRWIRIDTSCVCALSAAIGRT

SEQ ID NO: 28 - protein, 119 aa, Artificial Sequence
  Description of Artificial Sequence Synthetic polypeptide
YAEHKSHRGEASVCASESLWVTDKSSAIDIRGHQVTVLGEIKTGNSPVKQYFYETRCKEARPVKNGCRGIDDKHWNSQCKTSQTYVRALTSENNKLVGWRWIRIDTSCVCALSAAIGRT

SEQ ID NO: 29 - protein, 119 aa, Artificial Sequence
  Description of Artificial Sequence Synthetic polypeptide
YAEHKSHRGEYSVCASESLWVTDKSSAIDIRGHQVTVLGEIKTGNSPVKQAFYETRCKEARPVKNGCRGIDDKHWNSQCKTSQTYVRALTSENNKLVGWRWIAIDTSCVCALSRKIGRT

SEQ ID NO: 30 - protein, 119 aa, Artificial Sequence
  Description of Artificial Sequence Synthetic polypeptide
YAEHKSHRGEYSVCASESLWVTDKSSAIDIRGHQVTVLGEIKTGNSPVKQYFYETRCKEARPVKNGCRGIDDKHWNSQCKTSQTYVRALTSENNKLVGWRWIAIDTSCVCALSAAIGRT

SEQ ID NO: 31 - protein, 119 aa, Artificial Sequence
  Description of Artificial Sequence Synthetic polypeptide
YAEHKSHRGEYSVCASESLWVTDKSSAIDIRGHQVTVLGEIKTGNSPVKQYFYETRCKEARPVKNGCRGIDDKHWNSQCKTSQTYVRALTSENNKLVGWRWIRIDTSCVCALSRKIGRT

SEQ ID NO: 32 - protein, 119 aa, Artificial Sequence
  Description of Artificial Sequence Synthetic polypeptide
YAEHKSHRGEYSVCDSESLWVTDKSSAIDIRGHQVTVLGEIKTGNSPVKQYFYETRCKEARPVKNGCRGIDDKHWNSQCKTSQTYVRALTSENNKLVGWRWIRIDTSCVCALSAAIGRT

SEQ ID NO: 33 - protein, 119 aa, Artificial Sequence
  Description of Artificial Sequence Synthetic polypeptide
YAEHKSHRGEYSVCASESLWVTDKSSAIDIAGHQVTVLGEIKTGNSPVKQYFYETRCKEARPVKNGCRGIDDKHWNSQCKTSQTYVRALTSENNKLVGWRWIRIDTSCVCALSRKIGRT

SEQ ID NO: 34 - protein, 119 aa, Artificial Sequence
  Description of Artificial Sequence Synthetic polypeptide
YAEHKSHRGEYSVCASESLWVTDKSSAIDIAGHQVTVLGAIKTGNSPVKQYFYETRCKEARPVKNGCRGIDDKHWNSQCKTSQTYVRALTSENNKLVGWRWIRIDTSCVCALSRKIGRT